(19) United States
(12) Patent Application Publication
(10) Pub. No.: US 2006/0047123 A1
(43) Pub. Date: Mar. 2, 2006

(54) Mercaptoamides as histone deacetylase inhibitors

(76) Inventors: Saleh Ahmed, Oxford (GB); Celine Combet, Oxford (GB); Scott Cuthill, Oxford (GB); Graham Dawson, Oxford (GB); William Gattrell, Oxford (GB); Mario Lobell, Oxford (GB); Neil Pegg, Oxford (GB); Imaad Saba, Oxford (GB); Simon Swain, Oxford (GB); Claire Thomas, Oxford (GB); Graham Wynne, Oxford (GB)

Correspondence Address: OSI PHARMACEUTICALS, INC., 58 SOUTH SERVICE ROAD, MELVILLE, NY, 11747, US

(21) Appl. No.: 11/218,396

(22) Filed: Sep. 2, 2005

Related U.S. Application Data
(60) Provisional application No. 60/606,751, filed on Sep. 2, 2004.

(51) Int. Cl. C07D 333/22; C07D 277/18; C07D 263/02

(52) U.S. Cl. 546/290; 564/152; 548/190; 548/504; 549/76; 548/215

(57) ABSTRACT

Mercaptoamide compounds, represented by Formulas (IA), (IB), (IIA), and (IIB): 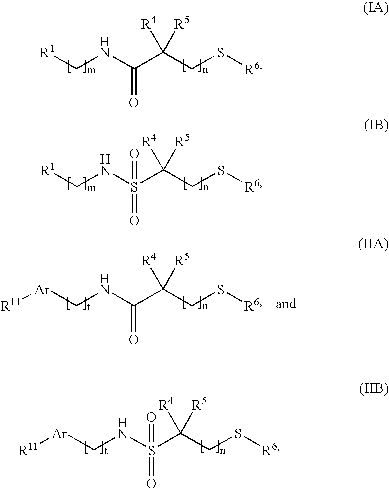 or a pharmaceutically acceptable salt thereof, inhibit histone deacetylase enzyme and are useful for the treatment and/or prevention of various infections, cancerous diseases, and conditions.

This application claims the benefit of U.S. Provisional Application No. 60/606,751 filed on Sep. 2, 2004, which is herein incorporated by reference in its entirety.

### BACKGROUND OF THE INVENTION

DNA in the nucleus of a cell comprises a compact complex of regular repeating structures called chromatin. Chromatin comprises repeating units of nucleosomes. The nucleosomes contain about 146 base pairs of DNA that are wound twice around a histone protein core. The histone proteins organized in the core are basic, highly conserved throughout evolution, and are identified as H2A, H2B, H3, and H4. When the DNA is wrapped around the protein core, the basic amino acids in the amino-terminal tails of the core histones interact with the negatively charged phosphate groups of the DNA. Covalent alterations of the histones at these amino-terminal tails by acetylation/deacetylation are enzymatically driven processes which are critical for modulating gene expression. See P. A. Marks et al., Nature Reviews, 1:194-202 (2001).

Acetylation of the tails of the histone proteins reduces the positive charge and causes the nucleosome to expand and facilitate the interaction of transcription factors to DNA. Deacetylation re-establishes the positive charge which causes the nucleosome to condense to a more compact structure. Thus, acetylation activates transcription of the DNA and encourages gene expression while deacetylation reverses the process and limits gene expression.

The amount of acetylation is controlled by two classes of enzymes, histone acetyl transferases (“HATs”) and histone deacetylases (also referred to as “HDA” or “HDACs”), both of which have activities that compete with each other to determine the pattern of histone acetylation and ultimately, to yield cell-specific patterns of gene expression. The enzymes' determination of the patterns of acetylation or deacetylation also controls cell cycle progression, differentiation, and/or apoptosis.

HDAC is a metallo-enzyme with zinc at the active site. Compounds having a zinc-binding moiety, such as a hydroxamic acid or phenylene diamine group, can inhibit HDAC. Some HDAC inhibitors are known to perform by fitting into the catalytic site of HDAC. This catalytic site has a tubular structure with the zinc atom at the base, and when the HDAC inhibitors fit into the site, the inhibitor binds to the zinc atom and limits acetylation of the histone proteins. Accordingly, histone deacetylase inhibition can repress gene expression, including expression of genes related to tumor suppression. C. M. Grozinger et al., Chemistry & Biology, 9:3-16 (2002) discusses HDACs and the mechanisms of HDAC inhibitors, and shows that there is great interest in research for inhibitors because they can inhibit a specific HDAC that is associated with a particular disease.

P. A. Marks et al., Journal of the National Cancer Institute, 92:1210-1216 (2000) and P. A. Marks et al., Nature Reviews, 1:194-202 (2001) describe how histone deacetylase inhibitors induce differentiation and/or apoptosis of transformed cells. S. W. Remiszewski, Current Opinion in Drug Discovery & Development, 5:487-499 (2002) shows advances in the discovery of small molecule histone deacetylase inhibitors.

Abnormal patterns of histone acetylation are linked to cancer, and HDAC inhibitors are known to have antiproliferative effects on tumor cells. HDAC inhibitors and pharmaceutical compositions thereof are known in the art to selectively and directly induce growth arrest, differentiation, and/or apoptotic cell death; to indirectly inhibit vascularisation of tumors; and are known to be active in vitro and in vivo. Such inhibitors can be extremely valuable as anticancer agents in treating conditions of, for example, transformed cell types including tumor types such as bladder, breast, ovarian, prostate, colon, lung, neuroblastoma, head and neck, and gliomas and hematological transformed cell lines such as lymphomas, leukemias, hemoglobinopathies, and multiple myeloma and genetic related metabolic disorders, such as cystic fibrosis and adrenoleukodystrophy. U.S. Pat. No. 6,428,983 B1 shows that HDAC inhibitors can also be used as an antiprotozoal agent to treat and/or prevent life threatening parasitic protozoal infections in animals and humans, such as malaria, toxoplasmosis, cryptosporoidiosis, trypanosomiasis, and coccidial infections.

W. K. Kelly et al., Expert Opin. Investig. Drugs, 11:1695-1713 (2002) describes histone deacetylase inhibitors, such as hydroxamic acid-based inhibitors, which are in clinical trials as anticancer agents. P. A. Marks et al., Current Opinion in Oncology, 13: 477-483 (2001) describes histone deacetylase inhibitors as cancer drugs and reviews those in clinical trials as well. M. L. Curtin, Expert Opin. Ther. Patents, 12:1375-1384 (2002) describes the patent status of known histone deacetylase inhibitors from patent literature from 1997 to mid-2002.

U.S. Pat. Nos. 6,495,719, 6,541,661, and 6,552,065 describe various histone deacetylase inhibitors. U.S. Pat. App. Pub. No. 2002/0192722 describes a sensor surface for detecting analytes comprising a reagent with a boronic acid complexing moiety. U.S. Pat. No. 6,462,179 describes the preparation of 1,2-phenylenediboronic acid bioconjugates for reagents and complexes for use as reagents to immobilize biologically active species. International Patent Publication No. WO2001007912 describes a hapten-polymer carrier complex used for immunoassays for pesticides and their degradation products. U.S. Pat. No. 6,333,325 describes a preparation of aromatic heterocyclic ureas as anti-inflammatory agents. International Patent Publication No. WO9813350 describes quinoline derivatives inhibiting the effect of growth factors such as VEGF. U.S. Pat. No. 6,414,148 and U.S. Pat. No. 6,184,225 describes the preparation of quinazoline derivatives and pharmaceutical compositions containing them by inhibiting the effects of VEGF. International Patent Publication No. WO9806696 describes the preparation of peptidyl compounds having MMP and TNF inhibitory activity. U.S. Pat. No. 6,265,411 describes oxindolylquinazoline derivatives as angiogenesis inhibitors. U.S. Pat. No. 6,514,971 describes a preparation of anilinocinnolines and related compounds as inhibitors of angiogenesis and vascular permeability. International Patent Publication No. WO9701275 describes a preparation of farnesyl-protein transferase inhibitor combinations to treat cancer. U.S. Pat. No. 5,470,997 describes amphetamine derivatives and protein and polypeptide amphetamine derivate conjugates and labels for preparing antibodies or receptors. International Patent Publication No. WO9302703 describes prodrugs useful as cytotoxic chemotherapeutic agents activated by targeted catalytic proteins. U.S. Pat. No. 5,136,034 describes a preparation of [(quinolylvinyl)phenyl]dithiaalkanedioates and analogs as leukotriene antagonists. International Patent Publication No. WO9111451 describes a preparation of griseolic acid analogs as LAK inhibitors and their pharmaceutical compositions used for treatment of viral hepatitis, autoimmune disorders, and rejection in organ transplantation. U.S. Pat. No. 5,030,726 describes cyclic ureas polymerizable to polymers bearing pendant urea groups. International Patent Publication No. WO2003013432 describes methods for sulfur-containing organic nitrate compounds used in the treatment and prevention of human diseases and conditions. JP2003034671 describes preparation of benzamides and their use as agrochemicals. International Patent Publication No. WO2002099077 describes methods and compositions related to tagging of membrane surface proteins. International Patent Publication No. WO2002098849 describes a preparation of peptide-related hydroxyalkylamines for pharmaceutical use in the treatment of Alzheimer's disease. International Patent Publication No. WO2002046129 describes a preparation of N-aryl, N-arylalkyl, and N-heterocyclylnonanamide and -octanamide derivatives and related compounds as inhibitors of histone deacetylase. International Patent Publication No. WO2002078947 describes sensor surfaces for detecting analytes. U.S. Pat. No. 6,462,179 describes a preparation of 1,2-phenylenediboronic acid bioconjugates for reagents and complexes. International Patent Publication No. WO2001007028 describes the use of retinoid receptor antagonists in the treatment of prostate carcinoma. International Patent Publication No. WO2000/043384 describes a preparation of aromatic heterocyclic ureas as anti-inflammatory agents. International Patent Publication No. WO2000/37451 describes a preparation of IL-5 inhibiting 6-azauracil derivatives. U.S. Pat. No. 6,043,026 describes a combination of growth hormone secretagogues and estrogen receptor modulators for the treatment of osteoporosis. U.S. Pat. No. 5,831,004 describes a preparation of inhibitors of metalloproteases and their pharmaceutical compositions. European Patent No. EP929526 describes quinoline derivatives inhibiting the effect of growth factors such as VEGF. European Patent No. EP925281 describes a preparation of peptidyl compounds having MMP and TNF inhibitory activity. U.S. Pat. No. 6,514,971 describes a preparation of anilinocinnolines and related compounds as inhibitors of angiogenesis and vascular permeability. U.S. Pat. No. 5,840,698 describes inhibitors of collagenase-1 and stromelysin-1 metalloproteases and their pharmaceutical compositions. U.S. Pat. No. 5,994,293 describes a preparation and therapeutic use of peptidyl sulfhydryl or acylthio compounds which inhibit metalloproteinase and TNF liberation. U.S. Pat. No. 5,536,716 describes spiro piperidines which promote release of growth hormone. U.S. Pat. No. 5,585,359 describes tetrapeptide-based inhibitors of farnesyl protein transferase. U.S. Pat. No. 5,457,194 describes substituted aliphatic amine-containing macrocyclic immunomodulators. International Patent Publication No. WO 1995/11029 describes a combination of bisphosphonates and growth hormone secretagogues for treatment of osteoporosis, and their preparation. International Patent Publication No. WO9305026 describes a preparation of peptide isosters containing a heterocycle as HIV inhibitors. International Patent Publication No. WO9302674 describes HIV protease inhibitors. U.S. Pat. No. 5,278,061 describes an affinity chromatography matrix useful in purifying interleukin-1 converting enzyme. U.S. Pat. No. 5,136,034 describes a preparation of [(quinolylvinyl)phenyl]dithiaalkanedioates and analogs as leukotriene antagonists.

However, there remains a need for potent HDAC inhibitors which are stable, efficacious, and inhibit tumor growth with little or no toxicity in order to have greater therapeutic potential.

### SUMMARY OF THE INVENTION

The present invention is directed to novel mercaptoamides, their salts, processes for their preparation, and compositions thereof as histone deacetylase inhibitors. The present invention is directed to compounds represented by Formulas (IA), (IIB), (IIA), and (IIB): 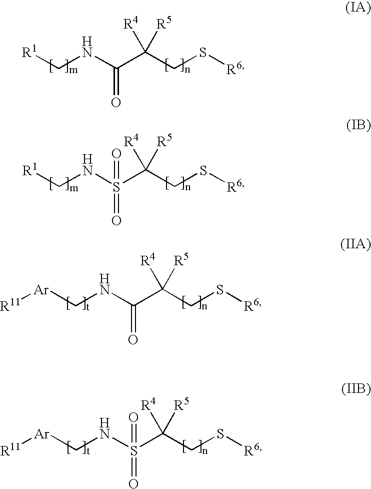
or a pharmaceutically acceptable salt thereof, which are useful in inhibiting histone deacetylase enzymes in animals, including humans, for the treatment and/or prevention of various infections, cancerous diseases, and conditions.

### DETAILED DESCRIPTION OF THE INVENTION

The present invention is directed to compounds represented by Formulas (IA), (IB), (IIA), and (IIB): 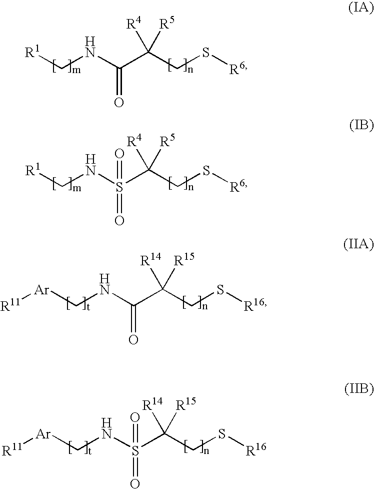
or a pharmaceutically acceptable salt thereof, wherein:
R¹ is R²NR³C(O)—, R²NHC(O)NH—, R²NHC(S)NH—, R²SO₂NH—, R²C(O)NH—;
R¹¹ is R²NR³C(O)(CH₂)₁₋₂—, R²NHC(O)NH(CH₂)₁₋₂—, R²NHC(S)NH(CH₂)₁₋₂, R²SO₂NH(CH₂)₁₋₂—, or R²C(O)NH(CH₂)₁₋₂—;
m is 4-6;
t is 1 or 2;
R² is a C₀₋₂alkyl, aryl, heteroaryl, carbocyclyl, -heteroaryl-heteroaryl, -heteroaryl-C₁₋₄alkyl, -heteroaryl-OCH₃, -heteroaryl-aryl-halogen, -heteroaryl-aryl, -aryl-aryl, -aryl-SCH₃, -aryl-OCH₃, -aryl-CF₃, -aryl-O—C₂alkyl-heterocyclyl, —C₃₋₁₀cycloallkyl-aryl, —C₀₋₂alkyl-heterocyclyl, —C₀₋₂alkyl-heteroaryl, —C₀₋₂alkyl-aryl, —C₀₋₁alkyl-heteroaryl, -aryl-OCH₂-aryl, -aryl-CH₂O-aryl, -aryl-carbonyl-aryl, -aryl-C(O)CH₃, -aryl-O-aryl, -aryl-O-heterocyclyl, -aryl-C₁₋₄alkyl, -aryl-O—C₂₋₃alkyl-N(CH₃)(CH₃), C₀₋₁alkyl-heterocyclyl-C₀₋₁alkyl, C₀₋₁alkyl-heteroaryl-C₀₋₁alkyl, -heterocyclyl, -heterocyclyl-aryl, -heterocyclyl-heteroaryl, -aryl-heterocyclyl, -aryl-heteroaryl, or —CH(aryl)(aryl), any of which is optionally substituted with one or more of R²² or R²²²;
R²² or R²²² are C₀₋₄alkyl, halogen, —OH, —CF₃, —SCH₃, —OCH₃, —NH₂, —O(CH₂)₂N(CH₃)(CH₃), —OCH₂-aryl, —O(CH₂)₂-heterocyclyl, —C(O)CH₃, —O-heterocyclyl, aryloxy-C₀₋₁alkyl-, aryl, or heterocyclyl;
R³ is C₀₋₁alkyl, or R² and R³ taken together form a heterocyclic or carbocyclic ring, any of which is optionally substituted with one or more independent C₀₋₄alkyl, halogen, —OH, —SCH₃, —OCH₃, —NH₂, aryl, or heterocyclyl substituents;
R⁴, R⁵, R¹⁴, and R¹⁵ are each independently C₀₋₄alkyl, phenyl, or fluorine;
n is 0 or 1; and
R⁶ and R¹⁶ are hydrogen, methyl, ethyl, phenyl, benzyl, or acetyl.

In an aspect of the present invention, a compound is represented by Formula IA, or a pharmaceutically acceptable sale thereof, wherein R¹ is R²NR³C(O)—, and the other variables are as described above.

In an embodiment of this aspect, a compound of the invention is represented by Formula IA, or a pharmaceutically acceptable salt thereof, wherein R² is —C₀₋₂alkyl-aryl optionally substituted by R²², and the other variables are as described above.

In another embodiment of this aspect, a compound of the invention is represented by Formula IA, or a pharmaceutically acceptable salt thereof, wherein R² is —C₀₋₂alkyl-heteroaryl optionally substituted with R²², and the other variables are as described above.

In another embodiment of the aspect, a compound of the invention is represented by Formula IA, or a pharmaceutically acceptable salt thereof, wherein R² is —C₀₋₂alkyl-heterocyclyl optionally substituted with R²², and the other variables are as described above.

In another embodiment of the aspect, a compound of this invention is represented by Formula IA, or a pharmaceutically acceptable salt thereof, wherein R² is a carbocyclyl optionally substituted with R²², and the other variables are as described above.

In still another embodiment of the aspect, a compound of this invention is represented by Formula IA, or a pharmaceutically acceptable salt thereof, wherein R² is a —CH(aryl)(aryl) optionally substituted with R²², and the other variables are as described above.

In a second aspect of the present invention, a compound is represented by Formula IA, or a pharmaceutically acceptable salt thereof, wherein R¹ is R²NR³C(O)—, and R² and R³ are taken together to form an optionally substituted ring, and the other variables are as described above.

In an embodiment of this second aspect, a compound of the invention is represented by Formula IA, or a pharmaceutically acceptable salt thereof, wherein R¹ is R²NR³C(O)—, wherein R² and R³ are taken together to form a heterocyclic ring optionally substituted by R²², and the other variables are as described above.

In an embodiment of this second aspect, a compound of the invention is represented by Formula IA, or a pharmaceutically acceptable salt thereof, wherein R¹ is R²NR³C(O)—, wherein R² and R³ are taken together to form an optionally substituted carbocyclic ring, and the other variables are as described above.

In a third aspect of the present invention, a compound is represented by Formula IA, or a pharmaceutically acceptable salt thereof, wherein R¹ is R²NHC(O)NH—, and the other variables are as described above.

In a fourth aspect of the present invention, a compound is represented by Formula IA, or a pharmaceutically acceptable salt thereof, wherein R¹ is R²NHC(S)NH—, and the other variables are as described above.

In a fifth aspect of the present invention, a compound is represented by Formula IA, or a pharmaceutically acceptable salt thereof, wherein R¹ is R²SO₂NH—, and the other variables are as described above.

In a sixth aspect of the present invention, a compound is represented by Formula IA, or a pharmaceutically acceptable salt thereof, wherein R¹ is R₂C(O)NH—, and the other variables are as described above.

In a seventh aspect of the present invention, a compound is represented by Formulas IA, IB, IIA, or IIB, or a pharmaceutically acceptable salt thereof, wherein a homo-dimer of Formulas IA, IB, IIA, or IIB is present at the R⁶ or R¹⁶ position, and the other variables are as described above.

The compounds of the present invention include compounds represented by Formula IA below, or a pharmaceutically acceptable salt thereof, 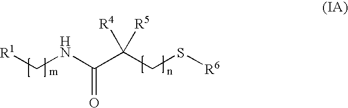
wherein R¹ is R²NR³C(O)—, R²NHC(O)NH—, R²NHC(S)NH—, R²SO₂NH—, or R²C(O)NH—;
m is 4-6;
R² is a C₀₋₂alkyl, aryl, heteroaryl, carbocyclyl, -heteroaryl-heteroaryl, -heteroaryl-C₁₋₄alkyl, -heteroaryl-OCH₃, -heteroaryl-aryl-halogen, -heteroaryl-aryl, -aryl-aryl, -aryl-SCH₃, -aryl-OCH₃, -aryl-CF₃, -aryl-O—C₂alkyl-heterocyclyl, —C₃₋₁₀cycloalkyl-aryl, —C₀₋₂alkyl-heterocyclyl, —C₀₋₂alkyl-heteroaryl, —C₀₋₂alkyl-aryl, —C₀₋₁alkyl-heteroaryl, -aryl-OCH₂-aryl, -aryl-CH₂O-aryl, aryl-carbonyl, -aryl-carbonyl-aryl, -aryl-C(O)CH₃, aryl-O-aryl, -aryl-O-heterocyclyl, -aryl-C₁₋₄alkyl, -aryl-O—C₂₋₃alkyl-N(CH₃)(CH₃), C₀₋₁alkyl-heterocyclyl-C₀₋₁alkyl, C₀₋₁alkyl-heteroaryl-C₀₋₁alkyl, -heterocyclyl, -heterocyclyl-aryl, -heterocyclyl-heteroaryl, -aryl-heterocyclyl, -aryl-heteroaryl, or —CH(aryl)(aryl), any of which is optionally substituted with R²²;
R²² is C₀₋₄alkyl, halogen, —OH, —CF₃, —SCH₃, —OCH₃, —NH₂, —O(CH₂)₂N(CH₃)(CH₃), —OCH₂-aryl, —O(CH₂)₂-heterocyclyl, —C(O)CH₃, —O-heterocyclyl, aryloxy-C₀₋₁alkyl-, aryl, or heterocyclyl;
R³ is C₀₋₁alkyl, or R² and R³ taken together form a heterocyclic or carbocyclic ring, any of which is optionally substituted with one or more independent C₁₋₄alkyl, halogen, —OH, —SCH₃, —OCH₃, —NH₂, aryl, or heterocyclyl substituents;
R⁴ and R⁵ are each independently C₀₋₄alkyl, phenyl, or fluorine;
n is 0 or 1; and
R⁶ is hydrogen, methyl, ethyl, phenyl, benzyl, or acetyl.

The compounds of the present invention include compounds represented by Formula IA above, or a pharmaceutically acceptable salt thereof, and
wherein R¹ is R²NR³C(O)—; or
wherein R¹ is R²NR³C(O)— and R² is —C₀₋₂alkyl-aryl which is optionally substituted with R²²; or
wherein R¹ is R²NR³C(O)— and R² is —C₀₋₂alkyl-heteroaryl which is optionally substituted with R²²; or
wherein R¹ is R²NR³C(O)— and R² is —C₀₋₂alkyl-heterocyclyl which is optionally substituted with R²²; or
wherein R¹ is R²NR³C(O)— and R² is a carbocyclyl which is optionally substituted with R²²; or
wherein R¹ is R²NR³C(O)— and R² is —CH(aryl)(aryl) which is optionally substituted with R²²; or
wherein R¹ is R²NR³C(O)— and R² and R³ are taken together to form a ring wherein said ring is optionally substituted with R²²; or
wherein R¹ is R²NR³C(O)— and R² and R³ are taken together to form a heterocyclic ring wherein said ring is optionally substituted with R²²; or
wherein R¹ is R²NR³C(O)— and R² and R³ are taken together to form a carbocyclic ring wherein said ring is optionally substituted with R²²; or
wherein R¹ is R²NHC(O)NH—; or
wherein R¹ is R²NHC(S)NH—; or
wherein R¹ is R²SO₂NH—; or
wherein R¹ is R₂C(O)NH—; or
wherein a homo-dimer of the compound is present at R⁶; and
wherein, in each case, the other variables are as defined as above for Formula IA.

The present invention includes a pharmaceutical composition comprising a therapeutically effective amount of a compound of Formula IA wherein a homo-dimer of the compound is present at R⁶, or a pharmaceutically acceptable salt thereof, and a pharmaceutically acceptable carrier.

The present invention includes a pharmaceutical composition comprising a therapeutically effective amount of a compound of Formula IA, or a pharmaceutically acceptable salt thereof, and a pharmaceutically acceptable carrier.

The present invention includes a method for treating cancerous diseases, infections, or metabolic disorders in a mammal by inhibiting histone deacetylase enzyme comprising administrating to said mammal a therapeutically effective amount of the compound of Formula IA, or a pharmaceutically acceptable salt thereof, wherein a homo-dimer of the compound is present at R⁶.

The present invention includes a method for treating cancerous diseases, infections, or metabolic disorders in a mammal by inhibiting histone deacetylase enzyme comprising administrating to said mammal a therapeutically effective amount of the compound of Formula IA, or a pharmaceutically acceptable salt thereof.

The present invention includes a method for treating cancerous diseases, infections, or metabolic disorders in a mammal by inhibiting histone deacetylase enzyme comprising administrating to said mammal a therapeutically effective amount of the pharmaceutical composition comprising a therapeutically effective amount of a compound Formula IA, or a pharmaceutically acceptable salt thereof, and a pharmaceutically acceptable carrier.

The present invention includes a method for treating cancerous diseases, infections, or metabolic disorders in a mammal by inhibiting histone deacetylase enzyme comprising administrating to said mammal a therapeutically effective amount of the compound of Formula IA, or a pharmaceutically acceptable salt thereof,
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes a method for treating cancerous diseases, infections, or metabolic disorders in a mammal by inhibiting histone deacetylase enzyme comprising administrating to said mammal a therapeutically effective amount of the compound of Formula IA, or a pharmaceutically acceptable salt thereof, wherein a homo-dimer of the compound is present at R⁶, and
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes a method for treating cancerous diseases, infections, or metabolic disorders in a mammal by inhibiting histone deacetylase enzyme comprising administrating to said mammal a therapeutically effective amount of a pharmaceutical composition comprising a therapeutically effective amount of a compound Formula IA, or a pharmaceutically acceptable salt thereof, and a pharmaceutically acceptable carrier,
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes a method for treating cancerous diseases, infections, or metabolic disorders in a mammal by inhibiting histone deacetylase enzyme comprising administrating to said mammal a therapeutically effective amount of a pharmaceutical composition comprising a therapeutically effective amount of a compound of Formula IA wherein a homo-dimer of the compound is present at R⁶, or a pharmaceutically acceptable salt thereof, and a pharmaceutically acceptable carrier, wherein
said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The compounds of the present invention include compounds represented by Formula IIB below, or a pharmaceutically acceptable salt thereof, 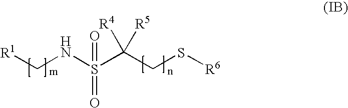
wherein R¹ is R²NR³C(O)—, R²NHC(O)NH—, R²NHC(S)NH—, R²SO₂NH—, or R²C(O)NH—;
m is 4-6;
R² is a C₀₋₂alkyl, aryl, heteroaryl, carbocyclyl, -heteroaryl-heteroaryl, -heteroaryl-C₁₋₄alkyl, -heteroaryl-OCH₃, -heteroaryl-aryl-halogen, -heteroaryl-aryl, -aryl-aryl, -aryl-SCH₃, -aryl-OCH₃, -aryl-CF₃, -aryl-O—C₂alkyl-heterocyclyl, —C₃₋₁₀cycloalkyl-aryl, —C₀₋₂alkyl-heterocyclyl, —C₀₋₂alkyl-heteroaryl, —C₀₋₂alkyl-aryl, —C₀₋₁alkyl-heteroaryl, -aryl-OCH₂-aryl, -aryl-CH₂O-aryl, aryl-carbonyl, -aryl-carbonyl-aryl, -aryl-C(O)CH₃, aryl-O-aryl, -aryl-O-heterocyclyl, -aryl-C₁₋₄alkyl, -aryl-O—C₂₋₃alkyl-N(CH₃)(CH₃), C₀₋₁alkyl-heterocyclyl-C₀₋₁alkyl, C₀₋₁alkyl-heteroaryl-C₀₋₁alkyl, -heterocyclyl, -heterocyclyl-aryl, -heterocyclyl-heteroaryl, -aryl-heterocyclyl, -aryl-heteroaryl, or —CH(aryl)(aryl), any of which is optionally substituted with R²²;
R²² is C₀₋₄alkyl, halogen, —OH, —CF₃, —SCH₃, —OCH₃, —NH₂, —O(CH₂)₂N(CH₃)(CH₃), —OCH₂-aryl, —O(CH₂)₂-heterocyclyl, —C(O)CH₃, —O-heterocyclyl, aryloxy-C₀₋₁alkyl-, aryl, or heterocyclyl;
R³ is C₀₋₁alkyl, or R² and R³ taken together form a heterocyclic or carbocyclic ring, any of which is optionally substituted with one or more independent C₁₋₄alkyl, halogen, —OH, —SCH₃, —OCH₃, —NH₂, aryl, or heterocyclyl substituents;
R⁴ and R⁵ are each independently C₀₋₄alkyl, phenyl, or fluorine;
n is 0 or 1; and
R⁶ is hydrogen, methyl, ethyl, phenyl, benzyl, or acetyl.

The present invention includes the compound of Formula IB, or a pharmaceutically acceptable salt thereof, wherein a homo-dimer of the compound is present at R⁶.

The present invention includes a pharmaceutical composition comprising a therapeutically effective amount of a compound of Formula IB, or a pharmaceutically acceptable salt thereof, and a pharmaceutically acceptable carrier.

The present invention includes a pharmaceutical composition comprising a therapeutically effective amount of a compound of Formula IB wherein a homo-dimer of the compound is present at R⁶, or a pharmaceutically acceptable salt thereof, and a pharmaceutically acceptable carrier.

The present invention includes a method for treating cancerous diseases, infections, or metabolic disorders in a mammal by inhibiting histone deacetylase enzyme comprising administrating to said mammal a therapeutically effective amount of the compound of Formula IB, or a pharmaceutically acceptable salt thereof, wherein a homo-dimer of the compound is present at R⁶.

The present invention includes a method for treating cancerous diseases, infections, or metabolic disorders in a mammal by inhibiting histone deacetylase enzyme comprising administrating to said mammal a therapeutically effective amount of the compound of Formula IB, or a pharmaceutically acceptable salt thereof.

The present invention includes a method for treating cancerous diseases, infections, or metabolic disorders in a mammal by inhibiting histone deacetylase enzyme comprising administrating to said mammal a therapeutically effective amount of the pharmaceutical composition comprising a therapeutically effective amount of a compound of Formula IB, or a pharmaceutically acceptable salt thereof, and a pharmaceutically acceptable carrier.

The present invention includes a method for treating cancerous diseases, infections, or metabolic disorders in a mammal by inhibiting histone deacetylase enzyme comprising administrating to said mammal a therapeutically effective amount of the compound of Formula IB, or a pharmaceutically acceptable salt thereof,
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes a method for treating cancerous diseases, infections, or metabolic disorders in a mammal by inhibiting histone deacetylase enzyme comprising administrating to said mammal a therapeutically effective amount of the compound of Formula IB, or a pharmaceutically acceptable salt thereof, wherein a homo-dimer of the compound is present at R⁶, and
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The presenting invention includes a method for treating cancerous diseases, infections, or metabolic disorders in a mammal by inhibiting histone deacetylase enzyme comprising administrating to said mammal a therapeutically effective amount of a pharmaceutical composition comprising a therapeutically effective amount of a compound of Formula IB, or a pharmaceutically acceptable salt thereof, and a pharmaceutically acceptable carrier, and
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes a method for treating cancerous diseases, infections, or metabolic disorders in a mammal by inhibiting histone deacetylase enzyme comprising administrating to said mammal a therapeutically effective amount of a pharmaceutical composition comprising a therapeutically effective amount of a compound of Formula IB wherein a homo-dimer of the compound is present at R⁶, or a pharmaceutically acceptable salt thereof, and a pharmaceutically acceptable carrier, and
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The compounds of the present invention include compounds represented by Formula IIA below, or a pharmaceutically acceptable salt thereof, 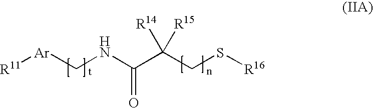
wherein Ar is aryl optionally substituted with one or more independent C₁₋₄alkyl, halogen, —OH, —SCH₃, —OCH₃, —NH₂, aryl, or heterocyclyl substituents;
R¹¹ is R¹²N¹³C(O)(CH₂)₁₋₂—, R¹²NHC(O)NH(CH₂)₁₋₂—, R¹²NHC(S)NH(CH₂)₁₋₂—, R¹²SO₂NH(CH₂)₁₋₂—, or R¹²C(O)NH(CH₂)₁₋₂—;
t is 1 or 2;
R¹² is a C₀₋₂alkyl, aryl, heteroaryl, carbocyclyl, -heteroaryl-heteroaryl, -heteroaryl-C₁₋₄alkyl, -heteroaryl-OCH₃, -heteroaryl-aryl-halogen, -heteroaryl-aryl, -aryl-aryl, -aryl-SCH₃, -aryl-OCH₃, -aryl-CF₃, -aryl-O—C₂alkyl-heterocyclyl, —C₃₋₁₀cycloalkyl-aryl, —C₀₋₂alkyl-heterocyclyl, —C₀₋₂alkyl-heteroaryl, —C₀₋₂alkyl-aryl, —C₀₋₁alkyl-heteroaryl, -aryl-OCH₂-aryl, -aryl-CH₂O-aryl, aryl-carbonyl, -aryl-carbonyl-aryl, -aryl-C(O)CH₃, aryl-O-aryl, -aryl-O-heterocyclyl, -aryl-C₁₋₄alkyl, -aryl-O—C₂₋₃alkyl-N(CH₃)(CH₃), C₀₋₁alkyl-heterocyclyl-C₀₋₁alkyl, C₀₋₁alkyl-heteroaryl-C₀₋₁alkyl, -heterocyclyl, -heterocyclyl-aryl, -heterocyclyl-heteroaryl, -aryl-heterocyclyl, -aryl-heteroaryl, or —CH(aryl)(aryl), any of which is optionally substituted with R²²²;
R²²² is C₀₋₄alkyl, halogen, —OH, —CF₃, —SCH₃, —OCH₃, —NH₂, —O(CH₂)₂N(CH₃)(CH₃), —OCH₂-aryl, —O(CH₂)₂-heterocyclyl, —C(O)CH₃, —O-heterocyclyl, aryloxy-C₀₋₁alkyl-, aryl, or heterocyclyl;
R¹³ is C₀₋₁alkyl, or R¹² and R¹³ taken together form a heterocyclic or carbocyclic ring, any of which is optionally substituted with one or more independent C₁₋₄alkyl, halogen, —OH, —SCH₃, —OCH₃, —NH₂, aryl, or heterocyclyl substituents;
R¹⁴ and R¹⁵ are each independently C₀₋₄alkyl, phenyl, or fluorine;
n is 0 or 1; and
R¹⁶ is hydrogen, methyl, ethyl, phenyl, benzyl, or acetyl.

The present invention includes the compound of Formula IIA, or a pharmaceutically acceptable salt thereof, wherein a homo-dimer of the compound is present at R¹⁶.

The present invention includes a pharmaceutical composition comprising a therapeutically effective amount of a compound of Formula IIA, or a pharmaceutically acceptable salt thereof, and a pharmaceutically acceptable carrier.

The present invention includes a pharmaceutical composition comprising a therapeutically effective amount of a compound of Formula IIA wherein a homo-dimer of the compound is present at R¹⁶, or a pharmaceutically acceptable salt thereof, and a pharmaceutically acceptable carrier.

The present invention includes a method for treating cancerous diseases, infections, or metabolic disorders in a mammal by inhibiting histone deacetylase enzyme comprising administrating to said mammal a therapeutically effective amount of the compound of Formula IIA, or a pharmaceutically acceptable salt thereof, wherein a homo-dimer of the compound is present at R¹⁶.

The present invention includes a method for treating cancerous diseases, infections, or metabolic disorders in a mammal by inhibiting histone deacetylase enzyme comprising administrating to said mammal a therapeutically effective amount of the compound of Formula IIA, or a pharmaceutically acceptable salt thereof.

The present invention includes a method for treating cancerous diseases, infections, or metabolic disorders in a mammal by inhibiting histone deacetylase enzyme comprising administrating to said mammal a therapeutically effective amount of the pharmaceutical composition comprising a therapeutically effective amount of a compound of Formula IIA, or a pharmaceutically acceptable salt thereof, and a pharmaceutically acceptable carrier.

The present invention includes a method for treating cancerous diseases, infections, or metabolic disorders in a mammal by inhibiting histone deacetylase enzyme comprising administrating to said mammal a therapeutically effective amount of the compound of Formula IIA, or a pharmaceutically acceptable salt thereof,
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes a method for treating cancerous diseases, infections, or metabolic disorders in a mammal by inhibiting histone deacetylase enzyme comprising administrating to said mammal a therapeutically effective amount of the compound of Formula IIA , or a pharmaceutically acceptable salt thereof, wherein a homo-dimer of the compound is present at R¹⁶, and
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes a method for treating cancerous diseases, infections, or metabolic disorders in a mammal by inhibiting histone deacetylase enzyme comprising administrating to said mammal a therapeutically effective amount of a pharmaceutical composition comprising a therapeutically effective amount of a compound of Formula IIA, or a pharmaceutically acceptable salt thereof, and a pharmaceutically acceptable carrier, and
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes a method for treating cancerous diseases, infections, or metabolic disorders in a mammal by inhibiting histone deacetylase enzyme comprising administrating to said mammal a therapeutically effective amount of a pharmaceutical composition comprising a therapeutically effective amount of a compound of Formula IIA wherein a homo-dimer of the compound is present at R¹⁶, or a pharmaceutically acceptable salt thereof, and a pharmaceutically acceptable carrier, and wherein
said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The compounds of the present invention include compounds represented by Formula IIB below, or a pharmaceutically acceptable salt thereof, 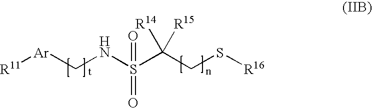
wherein R¹¹ is R¹²NR¹³C(O)(CH₂)₁₋₂—, R¹²NHC(O)NH(CH₂)₁₋₂—, R¹²NHC(S)NH(CH₂)₁₋₂, R¹²SO₂NH(CH₂)₁₋₂—, or R¹²C(O)NH(CH₂)₁₋₂—;
t is 1 or 2;
R¹² is a C₀₋₂alkyl, aryl, heteroaryl, carbocyclyl, -heteroaryl-heteroaryl, -heteroaryl-C₁₋₄alkyl, -heteroaryl-OCH₃, -heteroaryl-aryl-halogen, -heteroaryl-aryl, -aryl-aryl, -aryl-SCH₃, -aryl-OCH₃, -aryl-CF₃, -aryl-O—C₂alkyl-heterocyclyl, —C₃₋₁₀cycloalkyl-aryl, —C₀₋₂alkyl-heterocyclyl, —C₀₋₂alkyl-heteroaryl, —C₀₋₂alkyl-aryl, —C₀₋₁alkyl-heteroaryl, -aryl-OCH₂-aryl, -aryl-CH₂O-aryl, aryl-carbonyl, -aryl-carbonyl-aryl, -aryl-C(O)CH₃, aryl-O-aryl, -aryl-O-heterocyclyl, -aryl-C₁₋₄alkyl, -aryl-O—C₂₋₃alkyl-N(CH₃)(CH₃), C₀₋₁alkyl-heterocyclyl-C₀₋₁alkyl, C₀₋₁alkyl-heteroaryl-C₀₋₁alkyl, -heterocyclyl, -heterocyclyl-aryl, -heterocyclyl-heteroaryl, -aryl-heterocyclyl, -aryl-heteroaryl, or —CH(aryl)(aryl), any of which is optionally substituted with R²²²;
R²²² is C₁₋₄alkyl, halogen, —OH, —CF₃, —SCH₃, —OCH₃, —NH₂, —O(CH₂)₂N(CH₃)(CH₃), —OCH₂-aryl, —O(CH₂)₂-heterocyclyl, —C(O)CH₃, —O-heterocyclyl, aryloxy-C₀₋₁alkyl-, aryl, or heterocyclyl;
R¹³ is C₀₋₁alkyl, or R¹² and R¹³ taken together form a heterocyclic or carbocyclic ring, any of which is optionally substituted with one or more independent C₁₋₄alkyl, halogen, —OH, —SCH₃, —OCH₃, —NH₂, aryl, or heterocyclyl substituents;
R¹⁴ and R¹⁵ are each independently C₀₋₄alkyl, phenyl, or fluorine;
n is 0 or 1; and
R¹⁶ is hydrogen, methyl, ethyl, phenyl, benzyl, or acetyl.

The present invention includes the compound of Formula IIB, or a pharmaceutically acceptable salt thereof, wherein a homo-dimer of the compound is present at R¹⁶.

The present invention a pharmaceutical composition comprising a therapeutically effective amount of a compound of Formula IIB, or a pharmaceutically acceptable salt thereof, and a pharmaceutically acceptable carrier.

The present invention includes a pharmaceutical composition comprising a therapeutically effective amount of a compound of Formula IIB wherein a homo-dimer of the compound is present at R¹⁶, or a pharmaceutically acceptable salt thereof, and a pharmaceutically acceptable carrier.

The present invention includes a method for treating cancerous diseases, infections, or metabolic disorders in a mammal by inhibiting histone deacetylase enzyme comprising administrating to said mammal a therapeutically effective amount of the compound of Formula IIB, or a pharmaceutically acceptable salt thereof, wherein a homo-dimer of the compound is present at R¹⁶.

The present invention includes a method for treating cancerous diseases, infections, or metabolic disorders in a mammal by inhibiting histone deacetylase enzyme comprising administrating to said mammal a therapeutically effective amount of the compound Formula IIB, or a pharmaceutically acceptable salt thereof.

The present invention includes a method for treating cancerous diseases, infections, or metabolic disorders in a mammal by inhibiting histone deacetylase enzyme comprising administrating to said mammal a therapeutically effective amount of the pharmaceutical composition comprising a therapeutically effective amount of a compound of Formula IIB, or a pharmaceutically acceptable salt thereof, and a pharmaceutically acceptable carrier.

The present invention includes a method for treating cancerous diseases, infections, or metabolic disorders in a mammal by inhibiting histone deacetylase enzyme comprising administrating to said mammal a therapeutically effective amount of the compound Formula IIB, or a pharmaceutically acceptable salt thereof,
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes a method for treating cancerous diseases, infections, or metabolic disorders in a mammal by inhibiting histone deacetylase enzyme comprising administrating to said mammal a therapeutically effective amount of the compound of Formula IIB, or a pharmaceutically acceptable salt thereof, wherein a homo-dimer of the compound is present at R¹⁶, and
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes a method for treating cancerous diseases, infections, or metabolic disorders in a mammal by inhibiting histone deacetylase enzyme comprising administrating to said mammal a therapeutically effective amount of a pharmaceutical composition comprising a therapeutically effective amount of a compound of Formula IIB, or a pharmaceutically acceptable salt thereof, and a pharmaceutically acceptable carrier, and
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes a method for treating cancerous diseases, infections, or metabolic disorders in a mammal by inhibiting histone deacetylase enzyme comprising administrating to said mammal a therapeutically effective amount of the pharmaceutical composition comprising a therapeutically effective amount of a compound of Formula IIB wherein a homo-dimer of the compound is present at R¹⁶, or a pharmaceutically acceptable salt thereof, and a pharmaceutically acceptable carrier, and
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes use of a compound according to Formulas IA, IB, IIA, or IIB for the preparation of a pharmaceutical composition for the treatment of a cancerous disease, infection, or metabolic disorder in a mammal by inhibiting histone deacetylase enzyme.

The present invention includes use of a compound according to Formulas IA, IB, IIA, or IIB for the preparation of a pharmaceutical composition for the treatment of a cancerous disease, infection, or metabolic disorder in a mammal by inhibiting histone deacetylase enzyme,
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes use of a compound according to Formulas IA or IB, wherein a homo-dimer of the compound is present at R⁶, or use of a compound according to Formulas IIA or IIB, wherein a homo-dimer of the compound is present at R⁶, for the preparation of a pharmaceutical composition for the treatment of a cancerous disease, infection, or metabolic disorder in a mammal by inhibiting histone deacetylase enzyme.

The present invention includes use of a compound according to Formulas IA or IB, wherein a homo-dimer of the compound is present at R⁶, or use of a compound according to Formulas IIA or IIB, wherein a homo-dimer of the compound is present at R¹⁶, for the preparation of a pharmaceutical composition for the treatment of a cancerous disease, infection, or metabolic disorder in a mammal by inhibiting histone deacetylase enzyme,
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes use of a composition comprising a therapeutically effective amount of a compound of Formulas IA, IB, IIA, or IIB and a pharmaceutically acceptable carrier for the preparation of a pharmaceutical composition for the treatment of a cancerous disease, infection, or metabolic disorder in a mammal by inhibiting histone deacetylase enzyme.

The present invention includes use of a composition comprising a therapeutically effective amount of a compound of Formulas IA, IB, IIA, or IIB and a pharmaceutically acceptable carrier for the preparation of a pharmaceutical composition for the treatment of a cancerous disease, infection, or metabolic disorder in a mammal by inhibiting histone deacetylase enzyme,
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes use of a composition comprising a therapeutically effective amount of a compound of Formulas IA or IB, wherein a homo-dimer of the compound is present at R⁶, or a therapeutically effective amount of a compound according to Formulas IIA or IIB, wherein a homo-dimer of the compound is present at R¹⁶, and a pharmaceutically acceptable carrier for the preparation of a pharmaceutical composition for the treatment of a cancerous disease, infection, or metabolic disorder in a mammal by inhibiting histone deacetylase enzyme.

The present invention includes use of a composition comprising a therapeutically effective amount of a compound of Formulas IA or IB, wherein a homo-dimer of the compound is present at R⁶, or a therapeutically effective amount of a compound according to Formulas IIA or IIB, wherein a homo-dimer of the compound is present at R¹⁶, and a pharmaceutically acceptable carrier for the preparation of a pharmaceutical composition for the treatment of a cancerous disease, infection, or metabolic disorder in a mammal by inhibiting histone deacetylase enzyme,
wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

The present invention includes a compound selected from the group consisting of

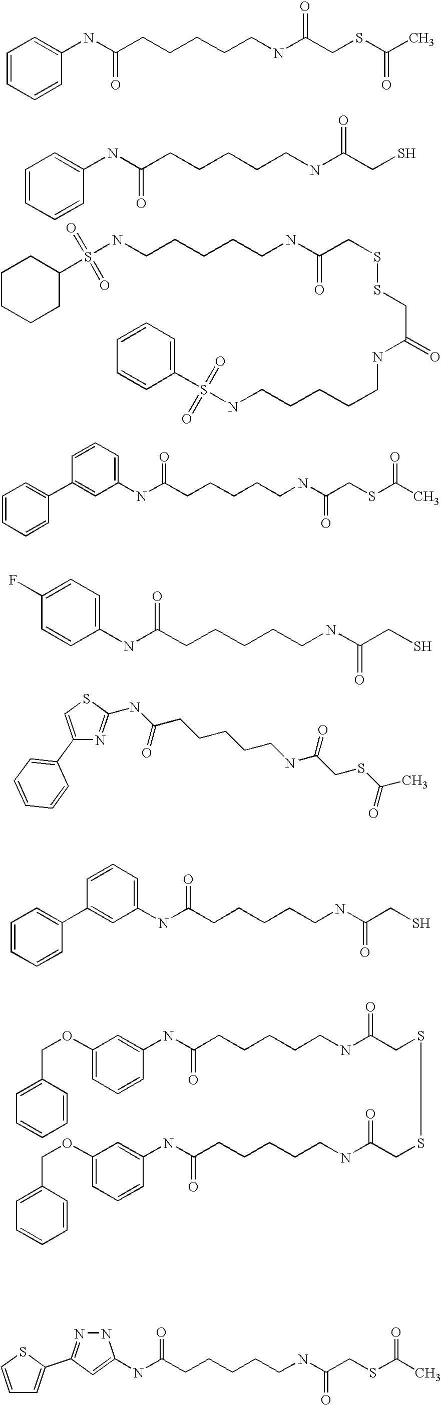

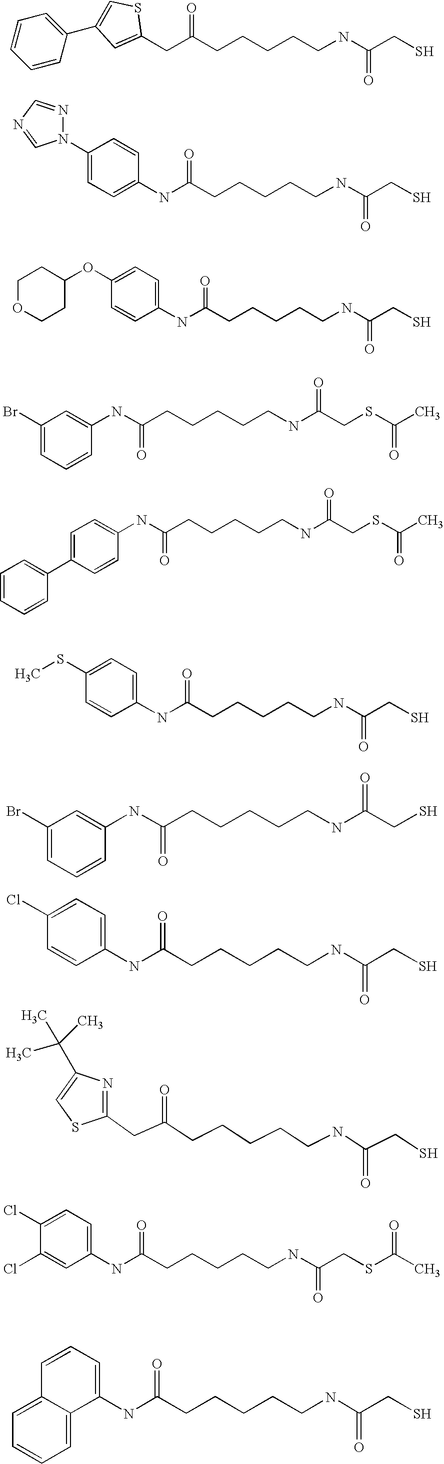

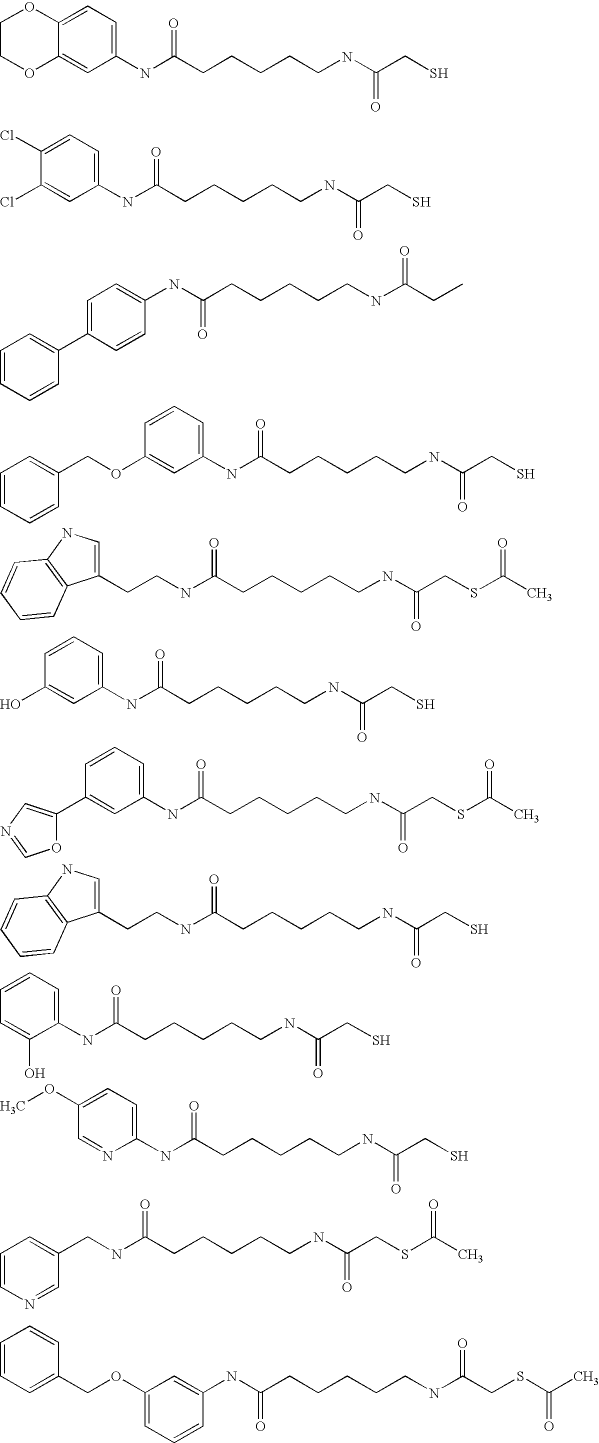

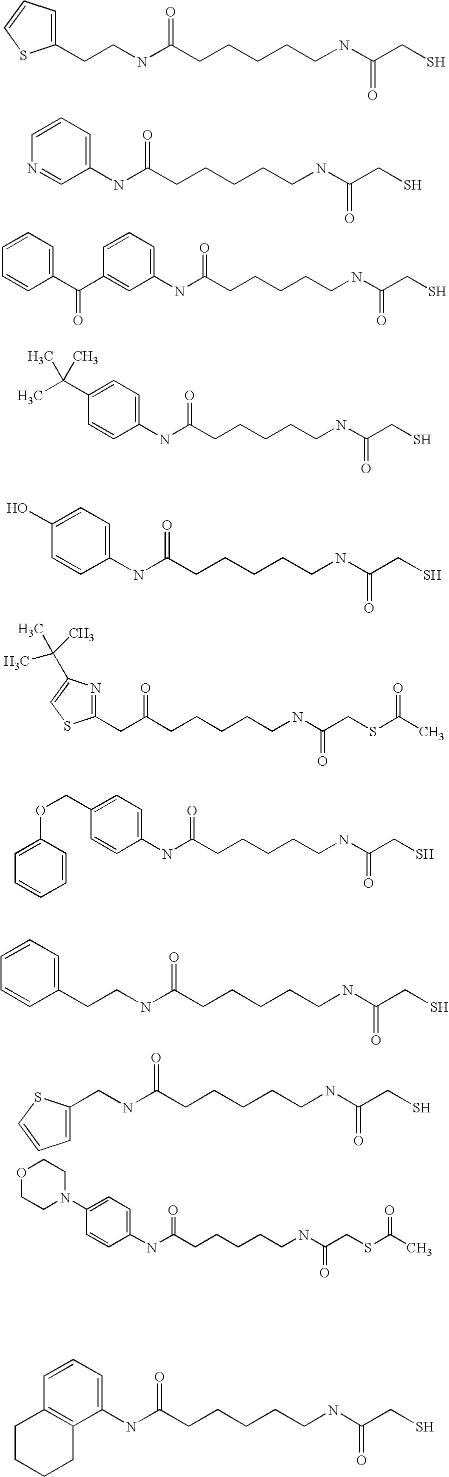

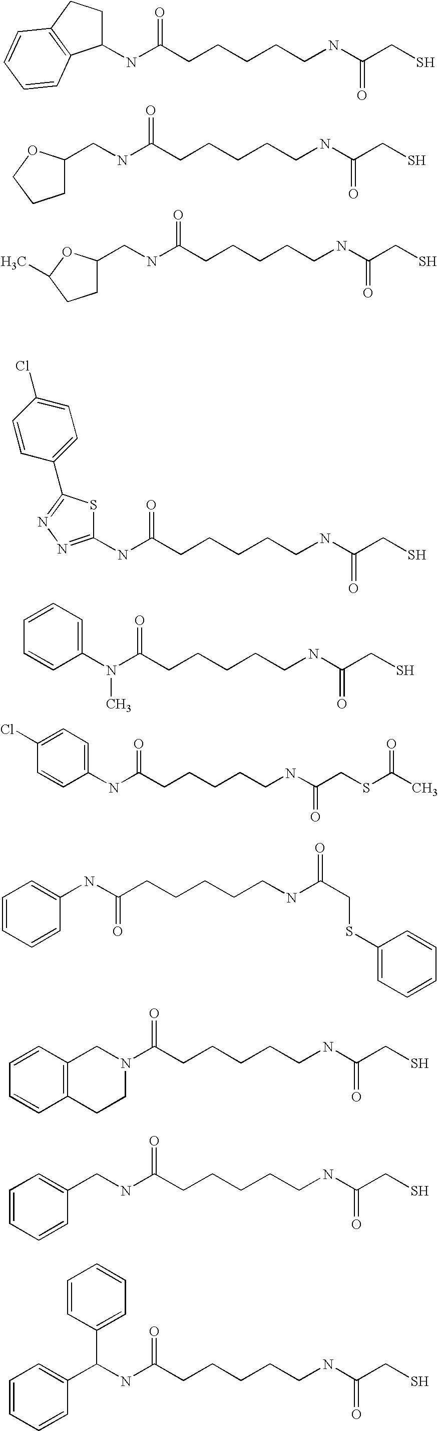

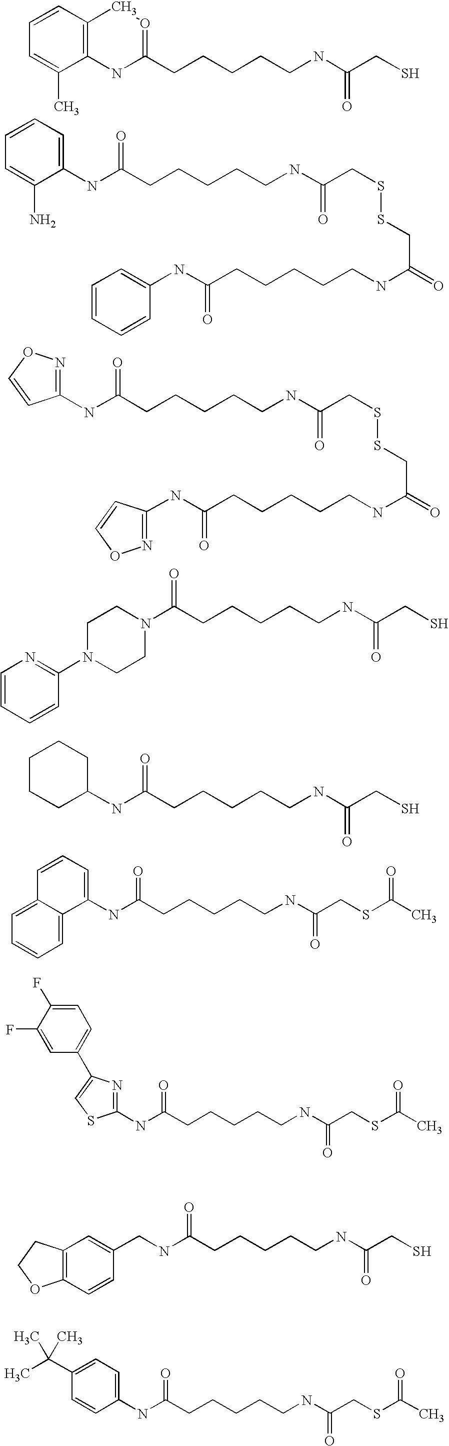

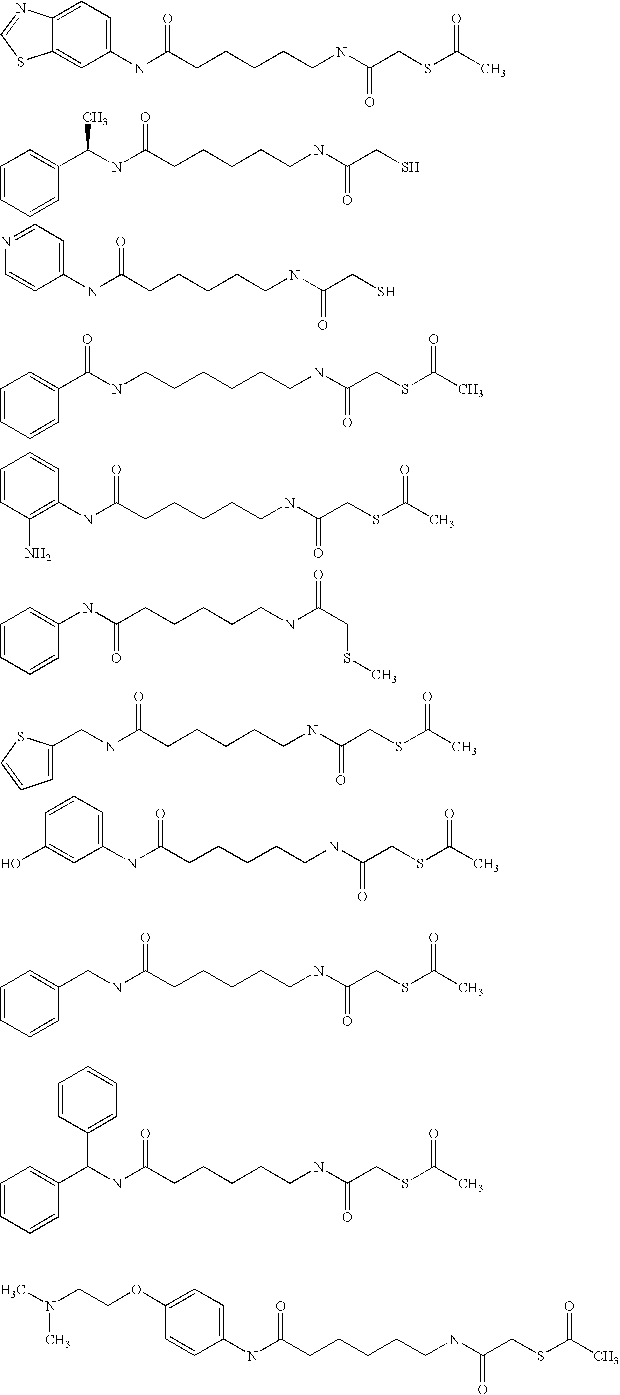

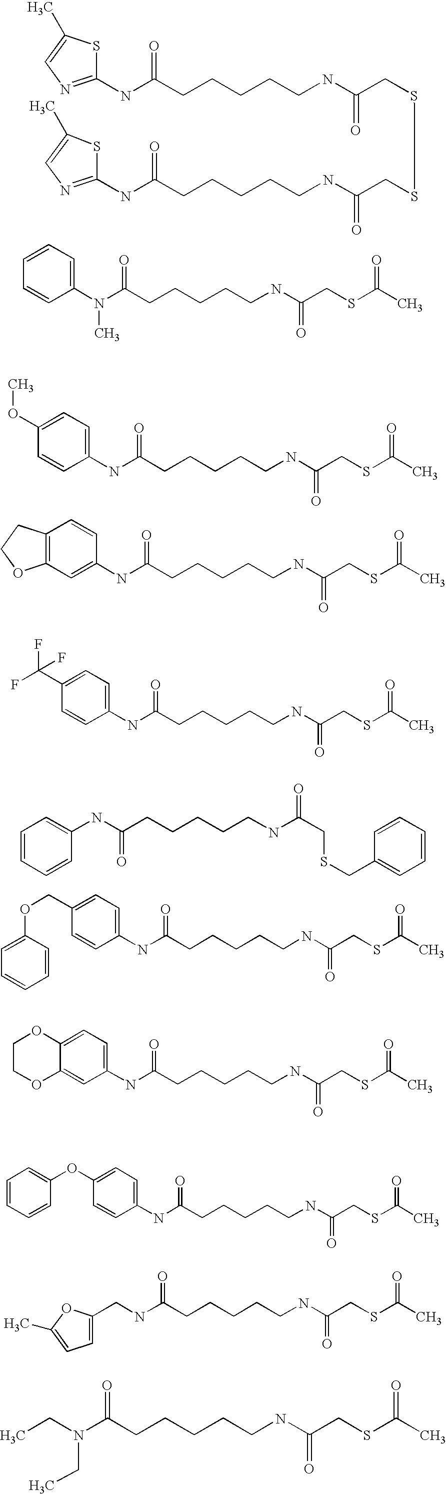

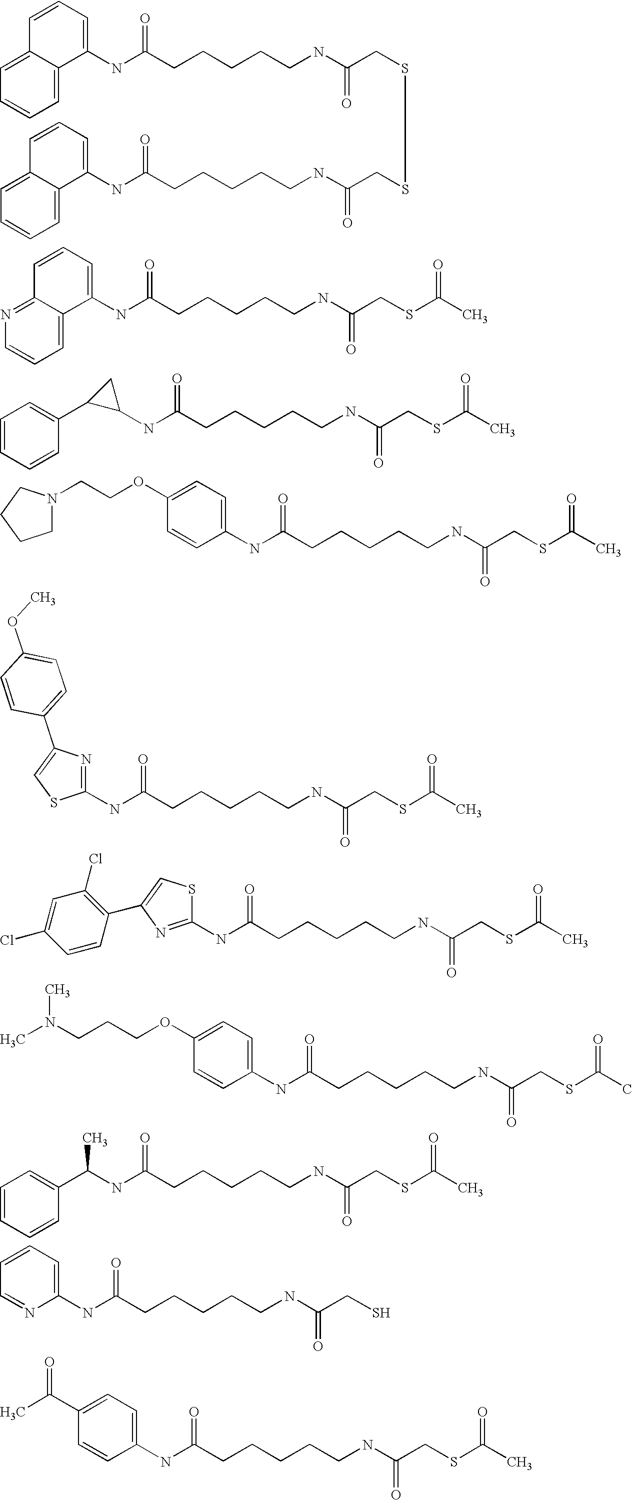

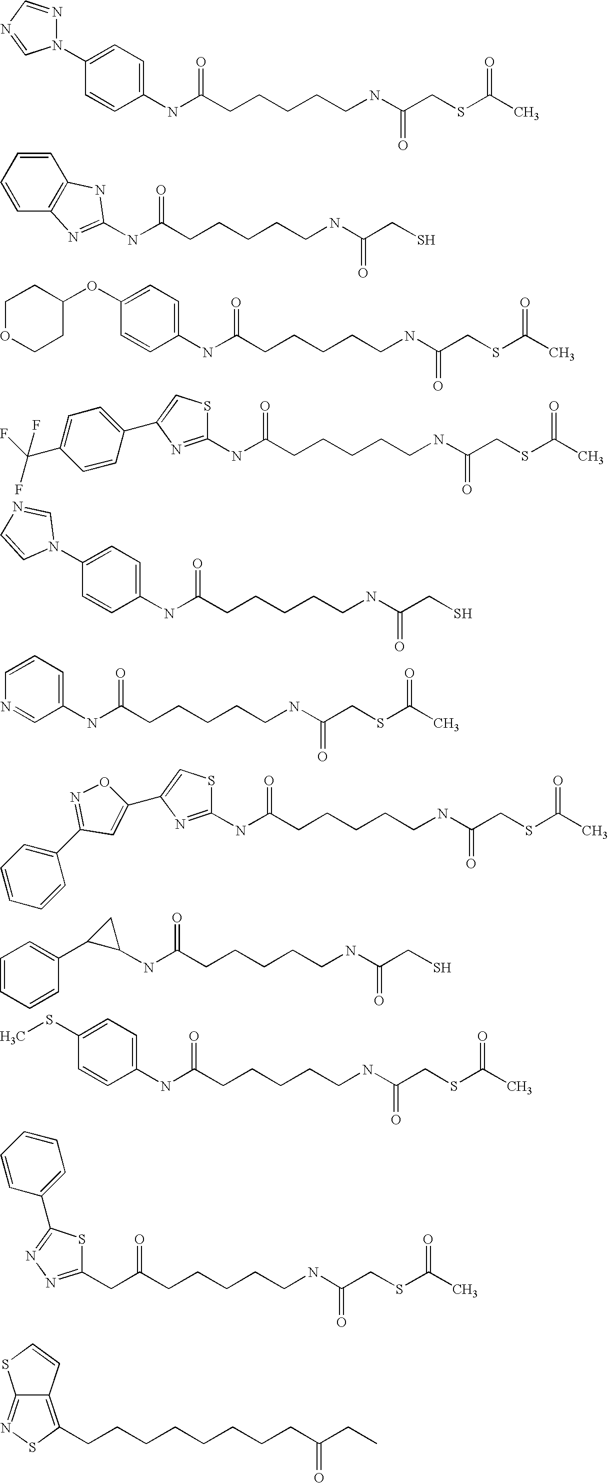

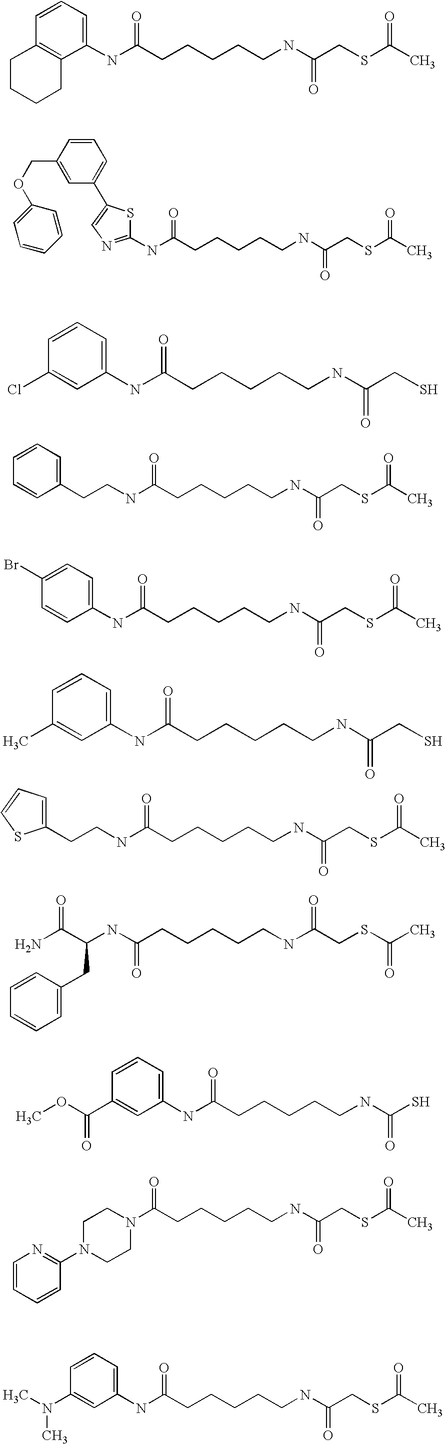

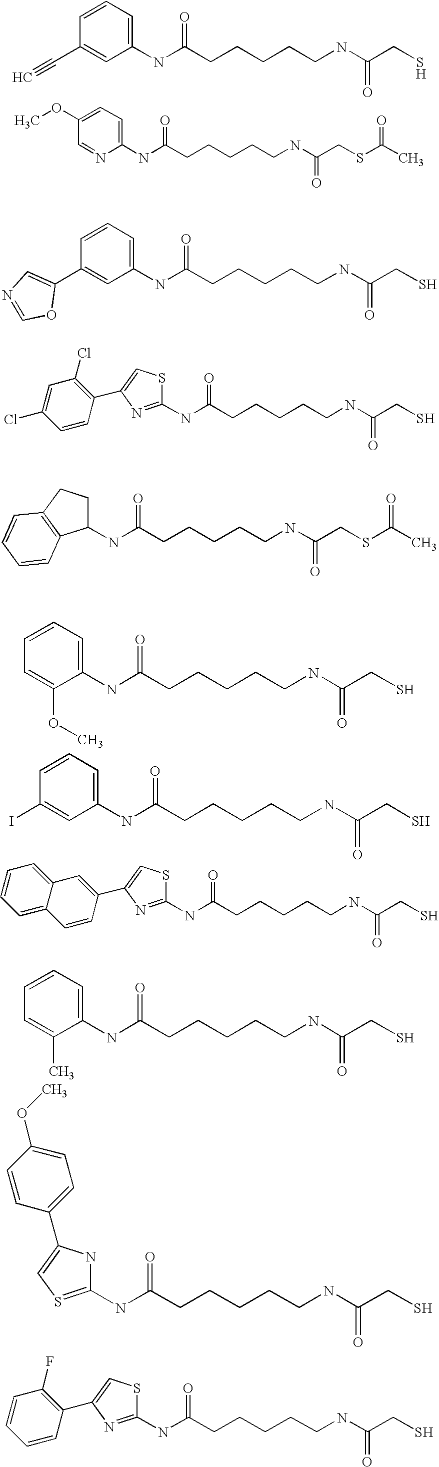

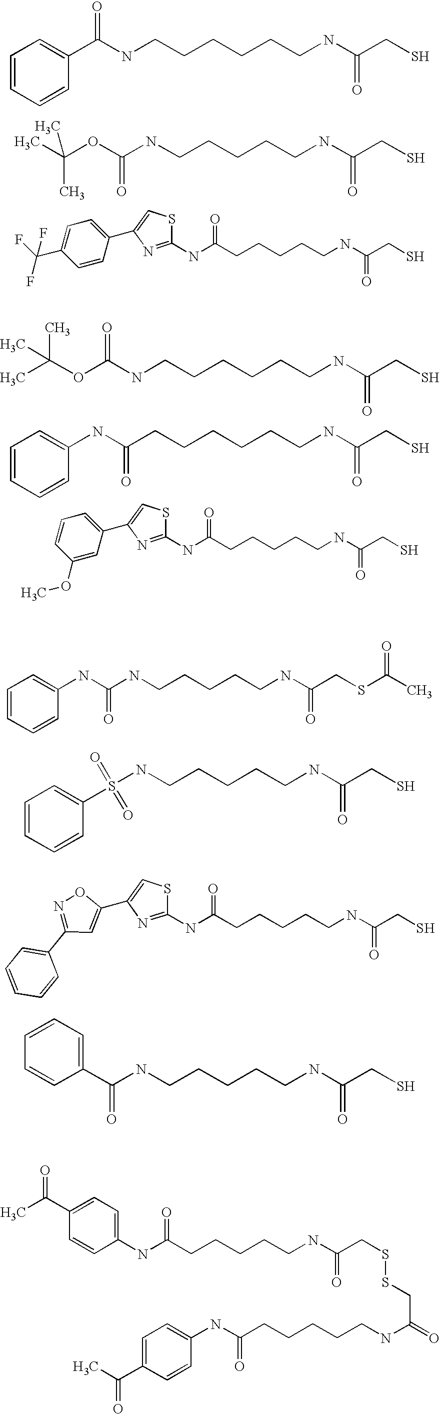

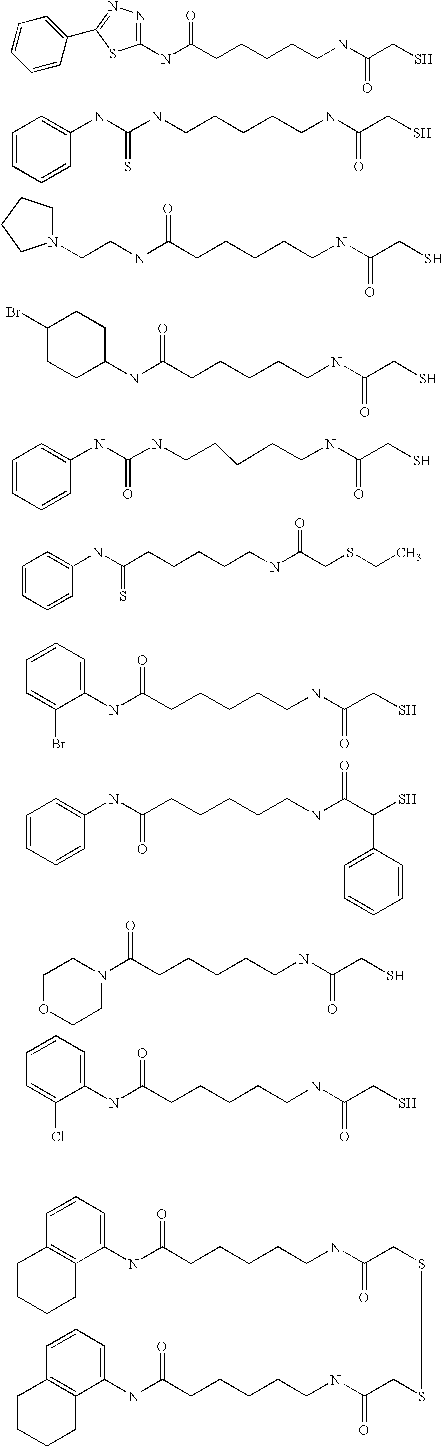

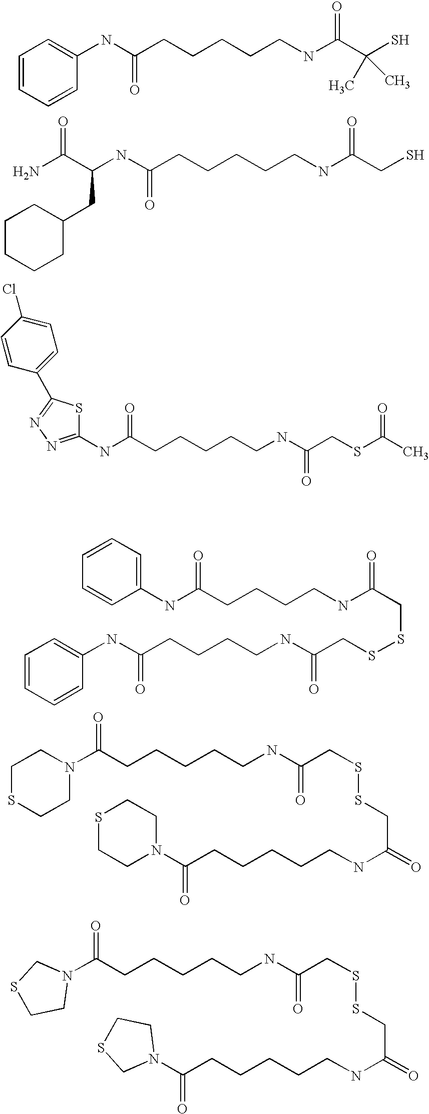

Unless otherwise stated, the connections of compound name moieties are at the rightmost recited moiety. That is, the substituent name starts with a terminal moiety, continues with any bridging moieties, and ends with the connecting moiety. For example, hetarylthioC₁₋₄alkyl has a heteroaryl group connected through a thio sulfur to a C₁₋₄ alkyl that connects to the chemical species bearing the substituent.

The term “C₀₋₆alkyl”, for example, is used to mean an alkyl having 6, 5, 4, 3, 2, 1, or no carbons—that is, 0, 1, 2, 3, 4, 5, or 6 carbons in a straight or branched configuration. An alkyl having no carbon atoms is a hydrogen atom substituent when the alkyl is a terminal group. An alkyl having no carbon atoms is a direct bond when the alkyl is a bridging (connecting) group.

As used herein, “alkyl” as well as other groups having the prefix “alk-” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated carbon-carbon bond.

The terms “carbocycle” or “carbocyclic” or “carbocyclyl” mean a cyclic aliphatic hydrocarbon ring structure which includes a single cycloalkane, cycloalkene, and cycloalkyne ring or a multiple ring system including the same or mixture of cycloalkane, cycloalkene, and cycloalkyne rings.

The terms “cycloalkyl” or “cyclyl” mean carbocycles containing no heteroatoms, and includes mono-, bi- and tri cyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyls and cyclyls include cyclopropyl, cyclobutyl, cyclopentyl, cyclohexyl, decahydronaphthalene, adamantane, indanyl, fluorenyl, 1,2,3,4-tetrahydronaphalene and the like. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, indenyl, and the like.

The terms “cycloalkyloxy” or “cyclyloxy”, unless specifically stated otherwise, includes a cycloalkyl group connected to the oxy connecting atom.

The term “alkoxy” unless specifically stated otherwise includes an alkyl group connected to the oxy connecting atom.

The term “aryl” unless specifically stated otherwise includes multiple ring systems as well as single ring systems such as, for example, phenyl or naphthyl.

The terms “aryloxy” or “aroxy”, unless specifically stated otherwise, includes multiple ring systems as well as single ring systems such as, for example, phenyl or naphthyl, connected through the oxy connecting atom to the connecting site.

The terms “hetero” or “het”, unless specifically stated otherwise, includes one or more O, S, or N atoms. For example, heterocycloalkyl, heterocyclyl, and heteroaryl include a substituted or unsubstituted saturated or unsaturated ring or multiple ring systems that contain one or more O, S, or N atoms in the ring, including mixtures of such atoms. The heteroatoms replace ring carbon atoms. Thus, for example, a heterocycloC₀₋₅alkyl is a five-membered ring containing from 5 to no carbon atoms.

Examples of “heterocyclyl” or “heterocycloalkyl” include, for example, azetidinyl, pyrrolidinyl, piperidinyl, piperazinyl, morpholinyl, tetrahydrofuranyl, imidazolinyl, pyrolidin-2-one, piperidin-2-one, thiomorpholinyl, tetrahydrofuryl, 4-pyranyl, tetrahydropyranyl, thiolanyl, dioxolanyl, dioxanyl, indolinyl, 5-methyl-6-chromanyl, oxetanyl, oxepanyl, oxocanyl, thietanyl, tetrahydrothiophenyl, tetrahydrothiopyranyl, [1,3]dioxanyl, oxazolidinyl, thiocanyl, thiepanyl, azepanyl, and azocanyl. Examples of “heteroaryl” or “hetaryl” include, for example, pyridinyl, quinolinyl, isoquinolinyl, pyridazinyl, pyrimidinyl, pyrazinyl, quinoxalinyl, furyl, benzofuryl, dibenzofuryl, thienyl, benzthienyl, pyrrolyl, indolyl, pyrazolyl, indazolyl, oxazolyl, isoxazolyl, thiazolyl, isothiazolyl, imidazolyl, benzimidazolyl, oxadiazolyl, thiadiazolyl, triazolyl, thiophenyl, and tetrazolyl.

The terms “heteroaryloxy” or “hetaryloxy” or “heteroaroxy”, unless specifically stated otherwise, describes a heteroaryl group connected through an oxy connecting atom to the connecting site.

Examples of “heteroarylC₁₋₆alkyl” or “hetarylC₁₋₆alkyl” include, for example, furylmethyl, furylethyl, thienylmethyl, thienylethyl, pyrazolylmethyl, oxazolylmethyl, oxazolylethyl, isoxazolylmethyl, thiazolylmethyl, thiazolylethyl, imidazolylmethyl, imidazolylethyl, benzimidazolylmethyl, oxadiazolylmethyl, oxadiazolylethyl, thiadiazolylmethyl, thiadiazolylethyl, triazolylmethyl, triazolylethyl, tetrazolylmethyl, tetrazolylethyl, pyridinylmethyl, pyridinylethyl, pyridazinylmethyl, pyrimidinylmethyl, pyrazinylmethyl, quinolinylmethyl, isoquinolinylmethyl and quinoxalinylmethyl.

Examples of arylC₁₋₆alkyl include, for example, phenylC₁₋₆alkyl, and naphthylC₁₋₆alkyl.

Examples of heterocycloC₃₋₇alkyl-carbonylC₁₋₆-alkyl include, for example, azetidinyl-carbonylC₁₋₆alkyl, pyrrolidinyl-carbonylC₁₋₆alkyl, piperidinyl-carbonylC₁₋₆alkyl, piperazinyl-carbonylC₁₋₆alkyl, morpholinyl-carbonylC₁₋₆alkyl, and thiomorpholinyl-carbonylC₁₋₆alkyl.

The term “amine” unless specifically stated otherwise includes primary, secondary and tertiary amines.

Unless otherwise stated, the term “carbamoyl” is used to include —NHC(O)OC₁₋₄alkyl, and —OC(O)NHC₁₋₄alkyl.

The term “halogen” includes fluorine, chlorine, bromine and iodine atoms.

The term “optionally substituted” is intended to include both substituted and unsubstituted. Thus, for example, optionally substituted aryl could represent a pentafluorophenyl or a phenyl ring. Further, the substitution can be made at any of the groups, at one or more of any of the groups, and with all the same or different substituents. For example, substituted arylC₁₋₆alkyl includes substitution on the aryl group as well as substitution on the alkyl group.

Compounds described herein contain one or more double bonds and may thus give rise to cis/trans isomers as well as other conformational isomers. The present invention includes all such possible isomers as well as mixtures of such isomers.

Compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formulas IA, IB, IIA, and IIB are shown without a definitive stereochemistry at certain positions. The present invention includes all stereoisomers of Formulas IA, IB, IIA, and IIB and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are benzenesulfonic, citric, hydrobromic, hydrochloric, maleic, phosphoric, sulfuric, and tartaric acids.

The pharmaceutical compositions of the present invention comprise a compound represented by Formulas IA, IB, IIA, or IIB (or pharmaceutically acceptable salts thereof) as an active ingredient, a pharmaceutically acceptable carrier and optionally other therapeutic ingredients or adjuvants. Such additional therapeutic ingredients include, for example, cytotoxic agents (alkylators, DNA topoisomerase inhibitors, antimetabolites, tubulin binders); inhibitors of angiogenesis; and other different forms of therapies including kinase inhibitors such as Tarceva, monoclonal antibodies, cancer vaccines, doxorubicin, vincristine, cisplatin, carboplatin, gemcitabine, and taxanes. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Creams, ointments, jellies, solutions, or suspensions containing the compounds of Formulas IA, IB, IIA, or IIB can be employed for topical use. Mouth washes and gargles are included within the scope of topical use for the purposes of this invention.

Dosage levels from about 0.001 mg/kg to about 140 mg/kg of body weight per day are useful in the treatment of conditions such as transformed cell types including solid tumor cell lines such as bladder, breast, ovarian, prostate, colon, lung, neuroblastoma, head and neck, and gliomas cancer and hematological transformed cell lines such as lymphomas, leukemias, hemoglobinopathies, multiple myeloma and genetic related metabolic disorders, such as cystic fibrosis and adrenoleukodystrophy, or as an antiprotozoal agent to treat and/or prevent life threatening parasitic protozoal infections in animals and humans, such as malaria, toxoplasmosis, cryptosporoidiosis, trypanosomiasis, and coccidial infections, or alternatively about 0.05 mg to about 7 g per patient per day. For example, inflammation may be effectively treated by the administration of from about 0.01 mg to 50 mg of the compound per kilogram of body weight per day, or alternatively about 0.5 mg to about 2.5 g per patient per day. Further, it is understood that the histone deacetylase inhibiting compounds of this invention can be administered at prophylactically effective dosage levels to prevent the above-recited conditions.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 0.01 mg to about 1000 mg of the active ingredient, typically 0.01 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In practice, the compounds represented by Formulas IA, IB, IIA, and IIB, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds represented by Formulas IA, IB, IIA, and IIB, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formulas IA, IB, IIA, and IIB. The compounds of Formulas IA, IB, IIA, and IIB, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formulas IA, IB, IIA, or IIB of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formulas IA, IB, IIA, and IIB, or pharmaceutically acceptable salts thereof, may also be prepared in powder or liquid concentrate form.

The compounds and pharmaceutical compositions of this invention have been found to exhibit biological activity as histone deacetylase inhibitors. Accordingly, another aspect of the invention is the treatment in mammals of, for example, angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; hematological transformed cell lines such as lymphomas, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, and multiple myeloma; genetic related metabolic disorders, such as cystic fibrosis and adrenoleukodystrophy; parasitic protozoal infections such as malaria, toxoplasmosis, cryptosporoidiosis, trypanosomiasis, and coccidial infections—by the administration of an effective amount of the compounds of this invention. The term “mammals” includes humans, as well as other animals such as, for example, dogs, cats, horses, pigs, and cattle. Accordingly, it is understood that the treatment of mammals other than humans is the treatment of clinical correlating afflictions to those above recited examples that are human afflictions.

Further, as described above, the compound of this invention can be utilized in combination with other therapeutic compounds. In particular, the combinations of the histone deacetylase inhibiting compound of this invention can be advantageously used in conjunction or combination with other such cancer therapeutic compounds. Such other compounds include, for example, a variety of cytotoxic agents (alkylators, DNA topoisomerase inhibitors, antimetabolites, tubulin binders); inhibitors of angiogenesis; and different other forms of therapies including kinase inhibitors such as Tarceva, monoclonal antibodies, and cancer vaccines. Other such compounds that can be beneficially co-administered with the compounds of the present invention include doxorubicin, vincristine, cisplatin, carboplatin, gemcitabine, and the taxanes. Thus, the compositions of the present invention include a compound according to Formulas IA, IB, IIA, or IIB, or a pharmaceutically acceptable salt thereof, and an anti-neoplastic, anti-tumor, anti-angiogenic, or chemotherapeutic agent.

The compounds of the present invention, or pharmaceutically acceptable salts thereof, can also be effectively administered in conjunction with other therapeutic compounds, aside from cancer therapy. For example, therapeutic agents effective to ameliorate adverse side-effects can be advantageous co-agents with the compounds of the present invention.

The compounds of the present invention, or pharmaceutically acceptable salts thereof, can also be effectively administered in conjunction with other cancer therapeutic compounds. For example, cytotoxic agents and angiogenesis inhibiting agents can be advantageous co-agents with the compounds of the present invention. Accordingly, the present invention includes compositions comprising the compounds represented by Formulas IA, IB, IIA, IIB, or a pharmaceutically acceptable salt thereof, and a cytotoxic agent or an angiogenesis-inhibiting agent. The amounts of each can be therapeutically effective alone—in which case the additive effects can overcome cancers resistant to treatment by monotherapy. The amounts of any can also be subtherapeutic—to minimize adverse effects, particularly in sensitive patients.

Compounds described herein contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formulas IA, IB, IIA, and IIB are shown without a definitive stereochemistry at certain positions. The present invention includes all stereoisomers of Formulas IA, IB, IIA, and IIB and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The invention also encompasses a pharmaceutical composition that is comprised of a compound of Formulas IA, IB, IIA, or IIB in combination with a pharmaceutically acceptable carrier.

Preferably the composition is comprised of a pharmaceutically acceptable carrier and a non-toxic therapeutically effective amount of a compound of Formulas IA, IB, IIA, or IIB as described above (or a pharmaceutically acceptable salt thereof).

Moreover, within this preferred embodiment, the invention encompasses a pharmaceutical composition for the treatment of disease by inhibiting histone deacetylase enzymes, resulting in acetylation/deacetylation of the HDAC which controls gene expression, cell cycle progression, differentiation, and/or apoptosis, comprising a pharmaceutically acceptable carrier and a non-toxic therapeutically effective amount of compound of Formulas IA, IB, IIA, or IIB as described above (or a pharmaceutically acceptable salt thereof).

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium slats. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, and tartaric acids.

The pharmaceutical compositions of the present invention comprise a compound represented by Formulas IA, IB, IIA, or IIB (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formulas IA, IB, IIA, or IIB, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration. E.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compounds represented by Formulas IA, IB, IIA, or IIB, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formulas IA, IB, IIA, or IIB. The compounds of Formulas IA, IB, IIA, or IIB, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical sue such as, for example, an aerosol, cream, ointment, lotion, dusting powder, or the like. Further, the compositions can be in a form suitable for use in transdermal devices. These formulations may be prepared, utilizing a compound represented by Formulas IA, IB, IIA, or IIB of this invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formulas IA, IB, IIA, or IIB, or pharmaceutically acceptable salts thereof, may also be prepared in powder or liquid concentrate form.

Generally, dosage levels on the order of from about 0.01 mg/kg to about 150 mg/kg of body weight per day are useful in the treatment of the above-indicated conditions, or alternatively about 0.5 mg to about 7 g per patient per day. For example, treating conditions of, for example, transformed cell types including solid tumor cell lines such as bladder, breast, ovarian, prostate, colon, lung, neuroblastoma, head and neck, and gliomas cancer and hematological transformed cell lines such as lymphomas, leukemias, hemoglobinopathies, and multiple myeloma and genetic related metabolic disorders, such as cystic fibrosis and adrenoleukodystrophy may be effectively accomplished by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination, and the severity of the particular disease undergoing therapy.

### EXPERIMENTAL

LCMS Method

Separation carried out on Waters Atlantis Column 2.1 mm×30 mm C18 3μ and Phenomenex Guard Column. Gradient supplied by Waters 1525 Pump and 4× Jasco PU-1585 Pumps. Autosampler: CTC, HTS PAL. UV Detector: Waters 2488 Mulitchannel UV detector at 220+254 nm. Mass Spec: Micromass MUX LCT. Detection: Cone Voltage 30v, Mass Range 80-700. System controlled using Masslynx 4.0 Software. Samples submitted using Openlynx Login v4.0 with data reported using Openlynx Browser v4.0
10 μL of sample approx 0.2 mg/ml injected.
Gradient: (a) H₂O+0.1% Formic Acid; (b) MeCN+0.1% Formic Acid
0-0.3 Mins -100% a
0.3-4.25 Mins 100% a to 10% a
0.25-4.40 Mins 10% a to 0% a
4.40-4.90 Mins 0% a to 0% a
4.90-5.00 Mins 0% a to 100% a
5.00-6.00 Mins 100% a

### Preparation of 6-(2-Acetylsulfanyl-acetylamino)-hexanamides

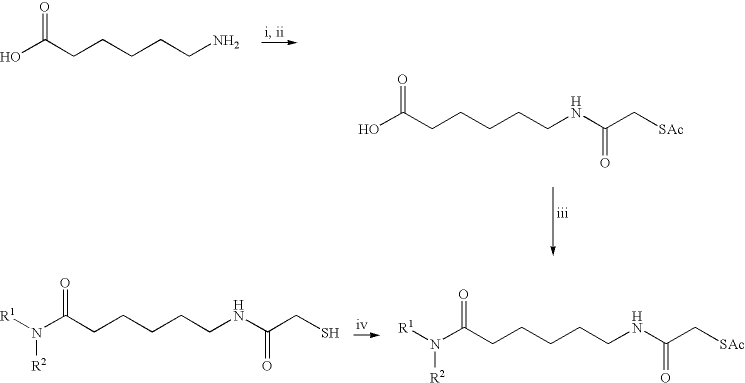 [0230] i. ClC(O)CH₂Cl, NaOH, DCM/H₂O; ii. KSAc, solvent; iii. HATU, DIPEA, THF; iv. NaOH, MeOH, then H⁺ or cHCl, MeOH

### Synthesis of 6-(2-Acetylsulfanyl-acetylamino)-hexanoic acid

To 5-amino-hexanoic acid (20 g, 0.153 moles) was added sodium hydroxide (7.34 g, 0.184 moles) in water (125 ml) and dichloromethane (250 ml). Chloroacetyl chloride (19 g, 0.168 moles) was added drop-wise over a period of 1 min. After 2 hr potassium thioacetate (19.72 g, 0.173 moles) was added, and stirring continued overnight. After this time, the organic phase was separated and washed with water, brine, dried and concentrated. The resulting solid was triturated with diethyl ether to afford 6-(2-Acetylsulfanyl-acetylamino)-hexanoic acid as an off-white solid. ¹H NMR (CDCl₃, 400 MHz) δ 1.40 (2H, m), 1.58 (2H, m), 1.69 (2H, m), 2.40 (2H, t), 2.56 (3H, s), 3.28 (2H, q), 3.59 (2H, s) and 6.24 (1H, br s).

### General procedure for preparation of 6-(2-Acetylsulfanyl-acetylamino)-hexanamides

To a stirred suspension of HATU (0.184 g, 0.485 mmol) and 6-(2-Acetylsulfanyl-acetylamino)-hexanoic acid (0.1 g, 0.4 mmol) in THF was added DIPEA (0.085 ml, 0.485 mmol). After 30 min the amine (0.485 mmol) was added and stirring continued overnight. After this time the reaction mixture was diluted was ethyl acetate (15 ml) and washed with water, saturated sodium hydrogen carbonate solution (2×10 ml), brine (10 ml), dried and concentrated. Solids were purified by trituration with ethyl acetate-diethyl ether; oils by flash chromatography.

Examples 138 and 152 were prepared in a similar manner to that described above, started instead from the heptanoic or pentanoic acid derivatives, respectively.

### General procedure for preparation of 6-(2-Mercapto-acetylamino)-hexanamides

General procedure A (Procedure Using Example 4)

To a stirred solution of thioacetic acid S-{[5-(biphenyl-3-ylcarbamoyl)-pentylcarbamoyl]-methyl} ester (52 mg, 0.13 mmol) in degassed methanol (3 ml) was added degassed 2M sodium hydroxide solution (75 μl, 0.13 mmol). After 30 mins, the solution was acidified by addition of DOWEX 50WX2-400 (pre-washed with water, methanol and acetone), filtered and concentrated to afford the 6-(2-Mercapto-acetylamino)-hexanoic acid biphenyl-3-ylamide. ¹H NMR (d₆-DMSO, 400 MHz) 1.38 (2H, m), 1.49 (2H, m), 1.63 (2H, m), 2.39 (2H, t), 2.71 (1H, t), 3.09 (4H, m), 7.36 (1H, m), 7.39 (2H, m), 7.49 (2H, t), 7.60 (3H, m), 7.94 (1H, br s), 7.99 (1H, br m) and 9.97 (1H, s). LCMS retention time: 3.40 min, Mf+=358.28.

General Procedure B (Procedure Using Example 13)

To a stirred solution of Thioacetic acid S-{[5-pyridin-3-yl carbamoyl)-pentylcarbamoyl]-methyl} ester (41 mg, 0.127 mmol) in degassed methanol (3 ml) was added degassed 2M sodium hydroxide solution (70 μl, 0.14 mmol). After 30 mins, the solution was diluted with water (5 ml) and extracted with ethyl acetate (10 ml). The aqueous layer was separated, neutralised by addition of 2M HCl solution and extracted with ethyl acetate (3×10 ml). The combined organics were washed with brine, dried and concentrated to afford the 6-(2-Mercapto-acetylamino)-hexanoic acid pyridin-3-ylamide. ¹H NMR (CD₃OD, 400 MHz), 1.49 (2H, m), 1.52 (2H, m), 1.55 (2H, m), 2.49 (2H, t), 3.15 (2H, s), 3.25 (2H, t), 7.43 (1H, dd), 8.15 (1H, d), 8.27 (1H, d) and 8.77 (1H, s). LCMS retention time: 2.31 min, MH⁺=283.11.

General Procedure C (Procedure Using Example 72)

To a solution of 6-(2-mercapto-acetylamino)-hexanoic acid naphthalene-1-ylamide (35 mg, 0.094 mmol) in methanol (2 ml) was added 2N ammonia in methanol (0.1 ml, 0.2 mmol). The flask was flushed with air and allowed to stir at rt for 1 hr. The mixture was concentrated, and the resulting solid triturated with methanol to afford 6-(2-{[5-naphthalene-1-ylcarbamoyl)-pentylcarbamoyl]-methyldisulfanyl}-acetylamino)-hexanoic acid naphthalene-1-ylamide as a colourless solid. LCMS retention time 3.49 min, MH⁺=660.28.

### Synthesis of N-[5-(2-mercapto-acetylamino)-alkyl derivatives]

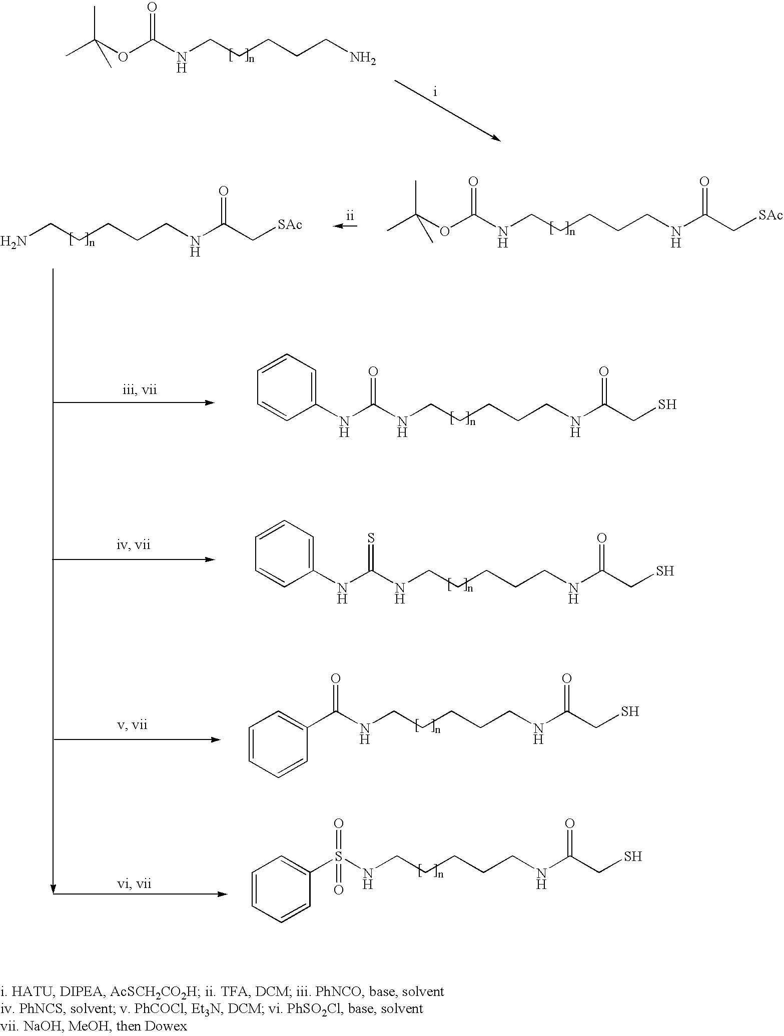

### Synthesis of thioacetic acid S-[(5-amino-pentylcarbamoyl)-methyl] ester trifluoroacetate (B, n=1)

To a stirred suspension of acetylsulfanyl-acetic acid (1.29 g, 9.62 mmol) and HATU (4.38 g, 11.5 mmol) in THF (20 ml) was added DIPEA (2.0 ml, 11.5 mmol). After 30 mins (5-amino-pentyl)-carbamic acid tert-butyl ester (2 ml, 9.61 mmol) was added, and stirring continued overnight. After this time the reaction mixture was diluted was ethyl acetate (25 ml) and washed with water (10 ml), 2N HCl (10 ml), saturated sodium hydrogen carbonate solution (2×20 ml), brine (20 ml), dried and concentrated. Trituration of the resulting brown solid with acetonitrile/diethylether afforded thioacetic acid S-[(5-tert-butoxycarbonylamino-pentylcarbamoyl)-methyl] ester. To a solution of thioacetic acid S-[(5-tert-butoxycarbonylamino-pentylcarbamoyl)-methyl] ester (A, 0.84 g, 2.52 mmol) in DCM (20 ml) was added trifluoroacetic acid (0.94 ml, 12.6 mmol). After stirring overnight the mixture was concentrated to afford thioacetic acid S-[(5-amino-pentylcarbamoyl)-methyl] ester trifluoroacetate. ¹H NMR (CDCl₃, 400 MHz) 1.44 (2H, m), 1.59 (2H, m), 1.74 (2H, m), 2.45 (3H, s), 3.09 (2H, m), 3.32 (2H, m) and 3.60 (2H, s).

### Synthesis of N-[5-(2-mercapto-acetylamino)-pentyl]-benzamide (Example 141)

To a stirred solution of benzoyl chloride (79 μL, 0.68 mmol) in DCM (2 ml) under argon was added triethylamine (0.12 ml, 0.9 mmol) followed by thioacetic acid S-[(5-amino-pentylcarbamoyl)-methyl] ester trifluroacetate (100 mg, 0.3 mmol) in DCM (1 ml) and stirring continued overnight. The reaction mixture was diluted with ethyl acetate (20 ml) and washed with water (5 ml), 2N HCl (5 ml), saturated sodium hydrogen carbonate solution (2×5 ml), brine (5 ml), dried and concentrated. The residue was taken into DCM (4 ml) and shaken with PS-Trisamine (603 mg, 3.38 mmol) for 5 hours. The resin was filtered, and the filtrate concentrated and triturated with ethyl acetate/diethyl ether to afford thioacetic acid S-[(5-benzoylamino-pentylcarbamoyl)-methyl] ester. LCMS Retention time: 2.86 min, MH⁺=322.14

Hydrolysis (Method A) followed by purification via SAX cartridge afforded N-[5-(2-mercapto-acetylamino)-pentyl]-benzamide (Example 141) as a yellow oil. ¹H NMR (CD₃OD, 400 MHz) 1.44 (2H, m), 1.57 (2H, m), 1.65 (2H, m), 3.12 (2H, s), 3.21 (2H, t), 3.38 (2H, t), 7.45 (2H, m), 7.54 (1H, m) and 7.81 (2H, m). LCMS retention time: 2.62 min, MH⁺=281.16.

### Synthesis of thioacetic acid S-[(5-benzenesulfonylamino-pentylcarbamoyl)-methyl] ester

To a stirred solution of phenylsulphonyl chloride (38 μL, 0.3 mmol) in DCM (2 mL) at 0° C. under argon was added DIPEA (0.1 ml, 0.6 mmol) followed by thioacetic acid S-[(5-amino-pentylcarbamoyl)-methyl] ester trifluroacetate (100 mg, 0.3 mmol) in DCM (1 ml). The mixture was allowed to warm to room temperature and stirring continued overnight. The mixture was diluted with ethyl acetate (20 ml) and washed with water (5 ml), 2N HCl (5 ml), saturated sodium hydrogen carbonate solution (2×5 ml), brine (5 ml), dried and concentrated. The residue was taken into DCM (2 ml) and shaken with PS-Trisamine (266 mg, 3.38 mmol) for 5 hours. The resin was filtered, and the filtrate concentrated and triturated with ethyl acetate /diethyl ether to afford thioacetic acid S-[(5-benzenesulfonylamino-pentylcarbamoyl)-methyl] ester.

### Synthesis of N-(5-benzenesulfonylamino-pentyl)-2-mercapto-acetamide (Example 140) and N-(5-benzenesulfonylamino-pentyl)-2-[(5-benzenesulfonylamino-pentylcarbamoyl)-methyldisulfanyl]-acetamide (Example 153)

Hydrolysis (Method A) followed by purification via SAX cartridge afforded N-(5-benzenesulfonylamino-pentyl)-2-[(5-benzenesulfonylamino-pentylcarbamoyl)-methyldisulfanyl]-acetamide (Example 153) as a colorless oil. LCMS retention time: 3.36 min, MH⁺=632.43

Further elution afforded N-(5-benzenesulfonylamino-pentyl)-2-mercapto-acetamide (Example 140) as a colourless oil. LCMS retention time 2.95 min, MH⁺=317.23.

### Synthesis of thioacetic acid S-{[5-(3-phenyl-ureido)-pentylcarbamoyl]-methyl} ester (Example 139) and 2-mercapto-N-[5-(3-phenyl-ureido)-pentyl]-acetamide (Example 143)

To a stirred solution of thioacetic acid S-[(5-amino-pentylcarbamoyl)-methyl] ester trifluroacetate (100 mg, 0.3 mmol) in DCM (2 ml) at 0° C. under argon was added triethylamine (0.04 ml, 0.3 mmol) followed by phenyl isocyanate (0.03 ml, 0.3 mmol). The mixture was allowed to warm to room temp. and stirring continued overnight. The reaction mixture was diluted with ethyl acetate (20 ml) and washed with water (5 ml), 2N HCl (5 ml), saturated sodium hydrogen carbonate solution (2×5 ml), brine (5 ml), dried and concentrated. The residue was triturated with ethyl acetate/diethyl ether to afford thioacetic acid S-{[5-(3-phenyl-ureido)-pentylcarbamoyl]-methyl} ester (Example 139) as a colourless solid. LCMS retention time: 2.95 min, MH⁺=338.28

Hydrolysis (Method A) followed by purification via SAX cartridge afforded 2-mercapto-N-[5-(3-phenyl-ureido)-pentyl]-acetamide (Example 143). LCMS retention time: 2.77 min, MH⁺=296.17.

### Synthesis of 2-mercapto-N-[5-(3-phenyl-thioureido)-pentyl]-acetamide (Example 142)

To a stirred solution of thioacetic acid S-[(5-amino-pentylcarbamoyl)-methyl] ester (100 mg, 0.3 mmol) trifluroacetate in DCM (2 ml) at 0° C. under argon was added triethylamine (0.04 ml, 0.3 mmol) followed by phenyl isothiocyanate (0.04 ml, 0.3 mmol). The mixture was allowed to warm to room temp and stirring continued overnight. The reaction mixture was diluted with ethyl acetate (20 ml) and washed with water (5 ml), 2N HCl solution (5 ml), saturated sodium hydrogen carbonate solution (2×5 ml), brine (5 ml), dried and concentrated. The residue was taken into DCM (2 ml) and shaken with PS-Trisamine (266 mg, 3.38 mmol) for 5 hours. The resin was filtered, and the filtrate concentrated and triturated with ethyl acetate/diethyl ether to afford thioacetic acid S-{[5-(3-phenyl-thioureido)-pentylcarbamoyl]-methyl} ester. Hydrolysis (Method A) followed by purification via SAX cartridge afforded 2-mercapto-N-[5-(3-phenyl-thioureido)-pentyl]-acetamide (Example 142). LCMS retention time: 2.81 min, MH⁺=312.15

### Synthesis of thioacetic acid S-[(6-benzoylamino-hexylcarbamoyl)-methyl] ester (Example 65) and N-[6-(2-mercapto-acetylamino)-hexyl]-benzamide (Example 135)

To a stirred suspension of acetylsulfanyl-acetic acid (1.0 g, 7.46 mmol) and HATU (3.40 g, 8.94 mmol) in THF (20 ml) was added DIPEA (1.5 ml, 8.60 mmol). After 30 mins (6-amino-hexyl)-carbamic acid tert-butyl ester hydrochloride (1.88 g, 7.43 mmol) was added, followed by further DIPEA (1.5 ml, 8.60 mmol) and stirring continued overnight. After this time the reaction mixture was diluted was ethyl acetate (25 ml) and washed with water (10 ml), 2N HCl (10 ml), saturated sodium hydrogen carbonate solution (2×20 ml), brine (20 ml), dried and concentrated. Trituration of the resulting brown solid with acetonitrile afforded thioacetic acid S-[(6-tert-butoxycarbonylamino-hexylcarbamoyl)-methyl] ester. ¹H NMR (d₆ DMSO, 400 MHz) 1.25 (4H, m), 1.36 (4H, m), 1.40 (9H, s), 2.37 (3H, s), 2.91 (2H, q), 3.05 (2H, q), 3.59 (2H, s), 6.78 (1H, br s) and 8.08 (1H, br s). To a solution of this (0.45 g, 1.15 mmol) in DCM (10 ml) was added trifluoroacetic acid (0.5 ml, 6.73 mmol). After stirring for 2 h the mixture was concentrated to afford thioacetic acid S-[(6-amino-hexylcarbamoyl)-methyl] ester trifluoroacetate as a colorless oil. To a stirred solution of benzoyl chloride (0.15 ml, 1.29 mmol) in DCM (3 ml) under argon was added triethylamine (0.25 ml, 1.79 mmol) followed by thioacetic acid S-[(6-amino-hexylcarbamoyl)-methyl] ester trifluroacetate (133 mg, 0.57 mmol) in DCM (1 ml) and stirring continued overnight. The reaction mixture was diluted with ethyl acetate (20 ml) and washed with water (5 ml), 2N HCl (5 ml), saturated sodium hydrogen carbonate solution (2×5 ml), brine (5 ml), dried and concentrated. The resulting brown solid was triturated with ethyl acetate/diethyl ether to afford thioacetic acid S-[(6-benzoylamino-hexylcarbamoyl)-methyl] ester (Example 65). LCMS retention time: 2.92 min, MH⁺=337.25

Hydrolysis (Method A) gave N-[6-(2-mercapto-acetylamino)-hexyl]-benzamide (Example 135) as an off-white solid. ¹H NMR (CD₃OD, 400 MHz) 1.46 (4H, m), 1.59 (2H, m), 1.67 (2H, m), 3.16 (2H, s), 3.23 (2H, t), 3.42 (2H, t), 7.48 (2H, t), 7.55 (1H, m) and 7.84 (2H, d). LCMS retention time: 2.81 min, MH⁺=295.24.
General procedure for synthesis of S-substituted derivatives

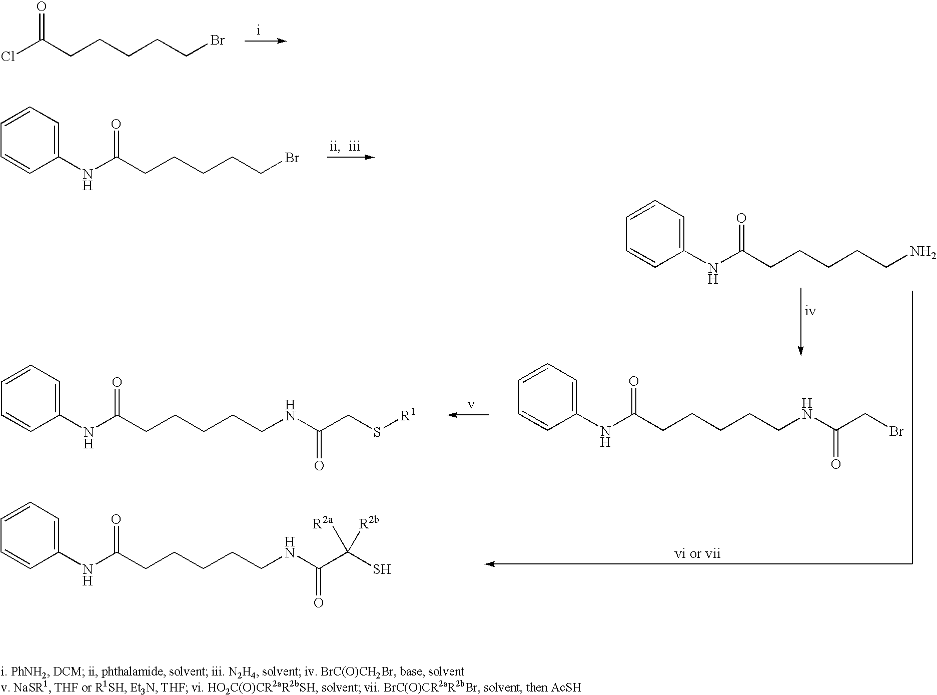

### Synthesis of 6-(2-bromo-acetylamino)-hexanoic acid phenylamide

To a stirred solution of 6-bromohexanoyl chloride (18.0 g, 84.3 mmol) in DCM (925 mL) at 0° C. under argon was added aniline (15.4 ml, 168.6 mmol) dropwise over 45 min and then stirred at this temperature for 1 hr. The mixture was allowed to warm to rt and stirring continued overnight. The reaction mixture was concentrated under reduced pressure and then diluted by ethyl acetate (500 ml) and then washed with 1M HCl (3×500 ml), dried and concentrated to give 6-bromo-hexanoic acid phenylamide (21.6 g) as a brown solid. LCMS retention time: 3.55 min, MH⁺=270.0, 272.0.

To a stirred solution of 6-bromo-hexanoic acid phenylamide (14.0 g, 51.8 mmol) in DMF (150 ml), potassium phthalamide (10.5 g, 57.0 mmol) was added and the reaction stirred overnight at rt. The reaction mixture was concentrated under reduced pressure and then diluted by ethyl acetate (500 ml) and then washed with water (2×500 ml), dried and concentrated to give the desired crude product which was purified by recrystallisation with ethyl acetate hexane to give 6-(1,3-dioxo-1,3-dihydro-isoindol-2-yl)-hexanoic acid phenylamide which was used directly in the next step. LCMS retention time: 3.67 min, MH⁺=337.2.

To a stirred solution of 6-(1,3-dioxo-1,3-dihydro-isoindol-2-yl)-hexanoic acid phenylamide (14.0 g, 51.8 mmol) in ethanol (150 ml) hydrazine hydrate (10.5 g, 57.0 mmol) was added and the reaction taken to reflux for 1.5 hrs. The reaction mixture was cooled and then concentrated under reduced pressure and then diluted by ethyl acetate (500 ml) and then washed with water (2×500 ml), dried and concentrated to 6-amino-hexanoic acid phenylamide. LCMS retention time: 2.47 min, MH+=207.2.

To a stirred suspension of 6-amino-hexanoic acid phenylamide (1.20 g, 5.82 mmol) in tetrahydrofuran (60 mL) and sodium carbonate (3.70 g, 34.90 mmol) at −10° C. under an inert atmosphere was added bromoacetyl bromide (0.76 mL, 8.73 mmol) in tetrahydrofuran (5 mL) and the resulting mixture was stirred at −10° C. for a further 2 h. The mixture was diluted with ethyl acetate (300 mL) then allowed to warm to rt. The mixture was washed with sat. sodium bicarbonate solution and brine then dried (sodium sulphate), filtered and evaporated under reduced pressure to afford an off-white solid. Crystallisation from dichloromethane/hexane afforded 6-(2-bromo-acetylamino)-hexanoic acid phenylamide. ¹H NMR (CDCl₃, 400 MHz) 1.44 (2H, m), 1.62 (2H, m), 1.81 (2H, m), 2.41 (2H, t), 3.38 (2H, m), 3.90 (2H, m), 6.60 (1H, br s), 7.14 (1H, m), 7.28 (3H, m), 7.58 (2H, d). LCMS retention time: 2.92 min, MH⁺=327.10, 329.10.

### Synthesis of 6-(2-methylsulfanyl-acetylamino)-hexanoic acid phenylamide (Example 85)

To a solution of 6-(2-bromo-acetylamino)-hexanoic acid phenylamide (50 mg, 0.15 mmol) in ethanol (5 ml) at room temperature was added sodium thiomethoxide (32 mg, 0.46 mmol), and the resulting thin suspension was stirred for 0.5 h. The reaction was then quenched by the addition of glacial acetic acid (0.1 ml), and the solution concentrated in vacuo. The resulting residue was partitioned between water and ethyl acetate, and the separated aqueous phase extracted with additional ethyl acetate. The combined organic phases were dried, filtered and evaporated to afford the title compound. LCMS retention time: 1.18 min, MH⁺=295.13

The following derivatives were prepared in an similar fashion: 6-(2-ethylsulfanyl-acetylamino)-hexanoic acid phenylamide (Example 149); 6-(2-phenylsulfanyl-acetylamino)-hexanoic acid phenylamide (Example 50) and 6-(2-benzylsulfanyl-acetylamino)-hexanoic acid phenylamide (Example 70).

### Assay

The protocol was adopted from a commercially available kit (BIOMOL). The source of HDAC enzyme was a crude extract of HDAC2 expressing T.ni insect cells. Acetylation of substrate was determined by adding the following reagents to wells in a 96 well plate. 5 μL vehicle or compound, 12.5 μL 80 μM substrate and 400 ng HDAC2 extract in assay buffer (25 mM Tris, 137 mM NaCl, 2.7 mM KCl, 1 mg/mL MgCl₂ pH 8.0) were mixed and incubated at rt for 2 h. The reaction was stopped by adding 25 mL of developer solution and plates read on a Molecular Devices FLEXstation fluorimeter) after 10 min by excitation at 360 nm and emission at 460 nm.

All Examples showed inhibition of histone deacetylase. The following Examples showed efficacy and activity by inhibiting histone deacetylase in the biochemical assay by having an IC₅₀ value of about 100 μM or less. It is preferred that the IC₅₀ value be less than about 50 μM. Even more preferred, the IC₅₀ value should be less than about 25 μM. Still more preferred, the IC₅₀ value should be less than 5 μM. Most preferred, the IC₅₀ value should be less than 1 μM.

| Example | Structure | Name | RT/min | MS_MH+ |
|---|---|---|---|---|
| 1 | 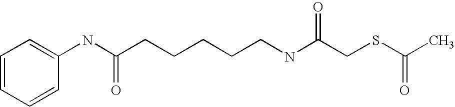 | Thioacetic acid S-[(5- phenylcarbamoyl- pentylcarbamoyl)-methyl]ester | 2.97 | 323.19 |
| 2 | 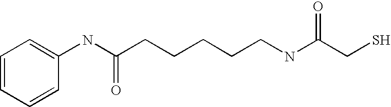 | 6-(2-Mercaptoacetylamino)- hexanoic acid phenylamide | 2.91 | 281.18 |
| 3 | 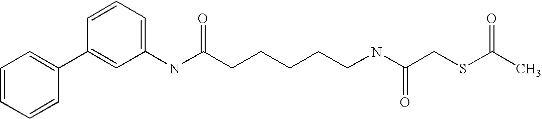 | Thioacetic acid S-{[5-(biphenyl-3- ylcarbamoyl)-pentylcarbamoyl]- methyl}ester | 3.44 | 399.15 |
| 4 | 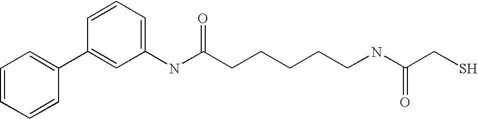 | 6-(2-Mercapto-acetylamino)- hexanoicacid biphenyl-3-ylamide | 3.4 | 357.27 |
| 5 | 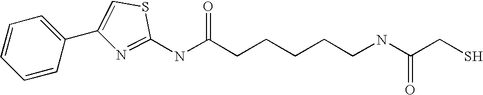 | 6-(2-Mercapto-acetylamino)- hexanoic acid (4-phenyl-thiazol-2- yl)-amide | 3.31 | 364.16 |
| 6 | 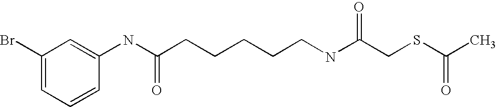 | Thioacetic acid S-{[5-(3-bromo- phenylcarbamoyl)- pentylcarbamoyl]-methyl}ester | 3.32 | 403.13 |
| 7 | 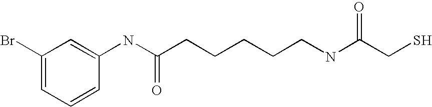 | 6-(2-Mercapto-acetylamino)- hexanoic acid (3-bromo-phenyl)- amide | 3.2 | 359.04 |
| 8 | 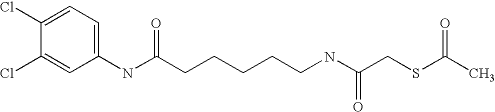 | Thioacetic acid S-{[5-(3,4- dichloro-phenylcarbamoyl)- pentylcarbamoyl]-methyl}ester | 3.41 | 391.05 |
| 9 | 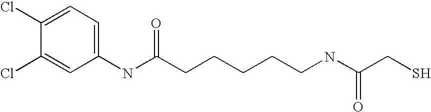 | 6-(2-Mercapto-acetylamino)- hexanoic acid (3,4-dichloro- phenyl)-amide | 3.4 | 349.05 |
| 10 | 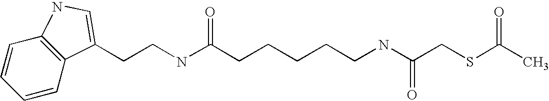 | Thioacetic acid S-({5-[2-(1H- indol-3-yl)-ethylcarbamoyl]- pentylcarbamoyl}-methyl) ester | 3.01 | 390.17 |
| 11 | 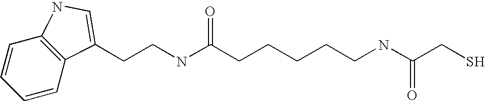 | 6-(2-Mercapto-acetylamino)- hexanoic acid [2-(1H-indol-3-yl)- ethyl]-amide | 1.24 | 348.14 |
| 12 | 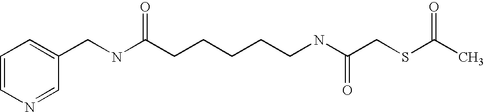 | Thioacetic acid S-({5-[(pyridin-3- ylmethyl)-carbamoyl]- pentylcarbamoyl}-methyl) ester | 0.79 | 338.15 |
| 13 | 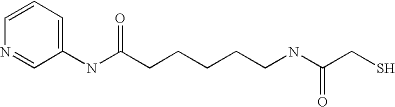 | 6-(2-Mercapto-acetylamino)- hexanoic acid pyridin-3-ylamide | 2.31 | 282.13 |
| 14 | 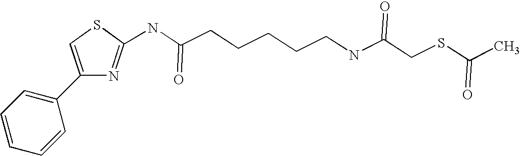 | Thioacetic acid S-{[5-(4-phenyl- thiazol-2-ylcarbamoyl)- pentylcarbamoyl]-methyl}ester | 3.45 | 406.16 |
| 15 | 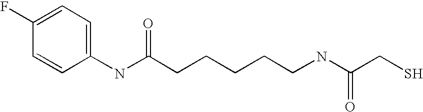 | 6-(2-Mercapto-acetylamino)- hexanoic acid (4-fluoro-phenyl)- amide | 2.98 | 299.23 |
| 16 | 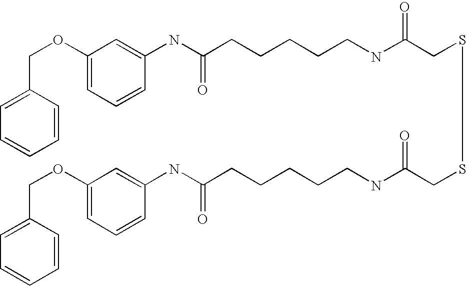 | 6-(2-{[5-(3-Benzyloxy- phenylcarbamoyl)- pentylcarbamoyl]- methyldisulfanyl}-acetylamino)- hexanoic acid (3-benzyloxy- phenyl)-amide | 4.03 | 771.57 |
| 17 | 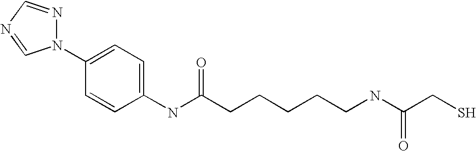 | 6-(2-Mercapto-acetylamino)- hexanoic acid (4-[1,2,4]triazol-1- yl-phenyl)-amide | 2.65 | 348.22 |
| 18 | 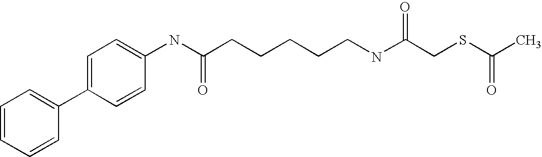 | Thioacetic acid S-{[5-(biphenyl-4- ylcarbamoyl)-pentylcarbamoyl]- methyl}ester | 1.54 | 399.04 |
| 19 | 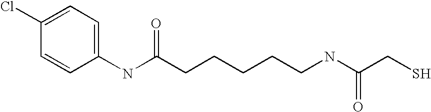 | 6-(2-Mercapto-acetylamino)- hexanoic acid (4-chloro-phenyl)- amide | 3.18 | 315.08 |
| 20 | 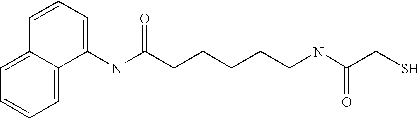 | 6-(2-Mercapto-acetylamino)- hexanoic acid naphthalen-1- ylamide | 1.28 | 331.12 |
| 21 | 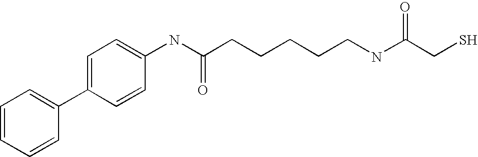 | 6-(2-Mercapto-acetylamino)- hexanoic acid biphenyl-4-ylamide | 3.39 | 357.16 |
| 22 | 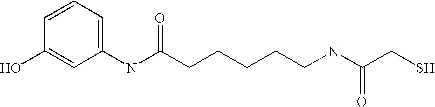 | 6-(2-Mercapto-acetylamino)- hexanoic acid (3-hydroxy-phenyl)- amide | 2.65 | 297.15 |
| 23 | 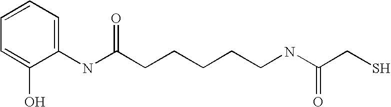 | 6-(2-Mercapto-acetylamino)- hexanoic acid (2-hydroxy-phenyl)- amide | 2.72 | 297.21 |
| 24 | 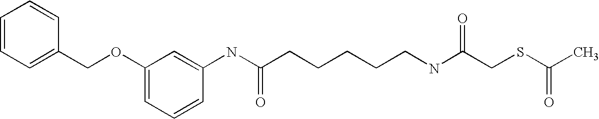 | Thioacetic acid S-{[5-(3- benzyloxy-phenylcarbamoyl)- pentylcarbamoyl]-methyl}ester | 3.56 | 429.27 |
| 25 | 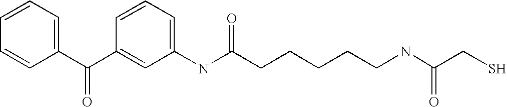 | 6-(2-Mercapto-acetylamino)- hexanoic acid (3-benzoyl-phenyl)- amide | 3.28 | 385.29 |
| 26 | 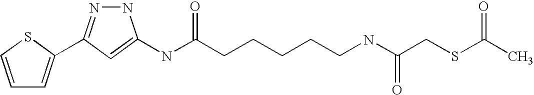 | Thioacetic acid S-{[5-(5-thiophen- 2-yl-2H-pyrazol-3-ylcarbamoyl)- pentylcarbamoyl]-methyl}ester | 3.4 | 395.09 |
| 27 | 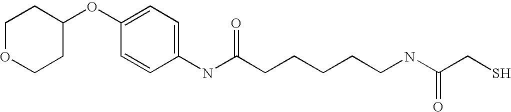 | 6-(2-Mercapto-acetylamino)- hexanoic acid [4-(tetrahydro- pyran-4-yloxy)-phenyl]-amide | 2.89 | 381.15 |
| 28 | 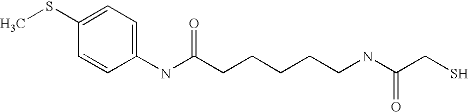 | 6-(2-Mercapto-acetylamino)- hexanoic acid (4-methylsulfanyl- phenyl)-amide | 3.15 | 327.22 |
| 29 | 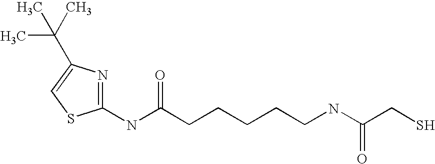 | 6-(2-Mercapto-acetylamino)- hexanoic acid (4-tert-butyl-thiazol- 2-yl)-amide | 3.27 | |
| 30 | 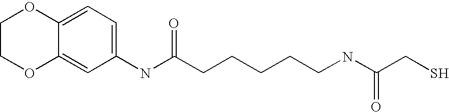 | 6-(2-Mercapto-acetylamino)- hexanoic acid (2,3-dihydro- benzo[1,4]dioxin-6-yl)-amide | 2.84 | 339.19 |
| 31 | 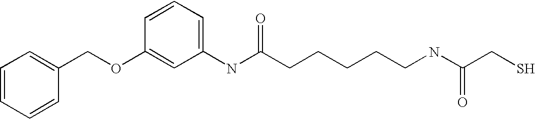 | 6-(2-Mercapto-acetylamino)- hexanoic acid (3-benzyloxy- phenyl)-amide | 3.42 | 387.2 |
| 32 | 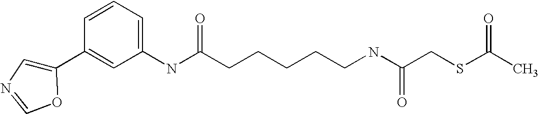 | Thioacetic acid S-{[5-(3-(5- oxazo)phenyl-carbamoyl)- pentylcarbamoyl]-methyl}ester | 1.24 | 390.04 |
| 33 | 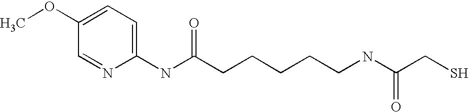 | 6-(2-Mercapto-acetylamino)- hexanoic acid (5-methoxy-pyridin- 2-yl)-amide | 2.64 | 312.17 |
| 34 | 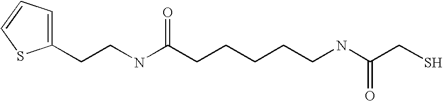 | 6-(2-Mercapto-acetylamino)- hexanoic acid (2-(thien-2-yl)ethyl)- amide | 2.85 | 315.18 |
| 35 | 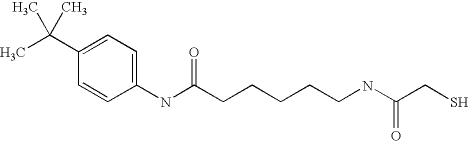 | 6-(2-Mercapto-acetylamino)- hexanoic acid (4-tert-butylphenyl)- amide | 3.39 | 337.15 |
| 36 | 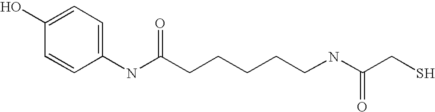 | 6-(2-Mercapto-acetylamino)- hexanoic acid (4-hydroxy-phenyl)- amide | 2.49 | 297.18 |
| 37 | 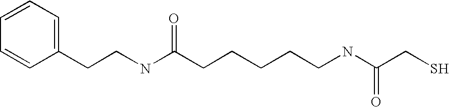 | 6-(2-Mercapto-acetylamino)- hexanoic acid phenethylamide | 1.2 | 309.16 |
| 38 | 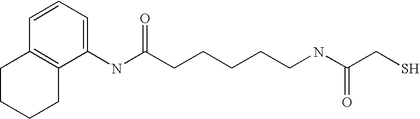 | 6-(2-Mercapto-acetylamino)- hexanoic acid (5,6,7,8- tetrahydronapht-1-yl)-amide | 3.26 | 335.26 |
| 39 | 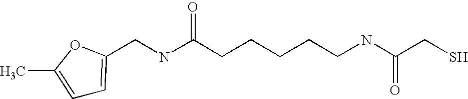 | 6-(2-Mercapto-acetylamino)- hexanoic acid (5-methylfurfuryl)- amide | 2.76 | 299.23 |
| 40 | 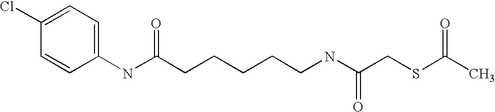 | Thioacetic acid S-{[5-(4- chlorophenyl-carbamoyl)- pentylcarbamoyl]-methyl}ester | 3.19 | 357.09 |
| 41 | 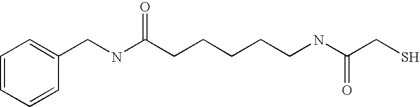 | 6-(2-Mercapto-acetylamino)- hexanoic acid benzylamide | 2.81 | 295.22 |
| 42 | 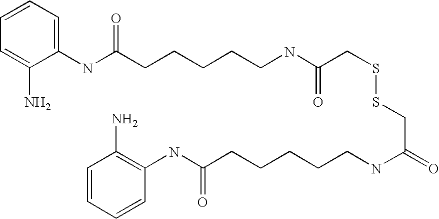 | 6-(2-{[5-(2-Amino- phenylcarbamoyl)- pentylcarbamoyl]- methyldisulfanyl}-acetylamino)- hexanoic acid (2-amino-phenyl)- amide | 2.61 | 589.36 |
| 43 | 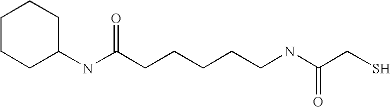 | 6-(2-Mercapto-acetylamino)- hexanoic acid cyclohexylamide | 2.84 | 287.21 |
| 44 | 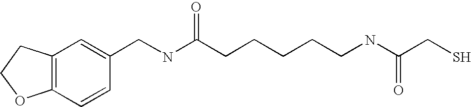 | 6-(2-Mercapto-acetylamino)- hexanoic acid (5-(2,3- dihydro[b]furan)methyl)-amide | 2.83 | 337.27 |
| 45 | 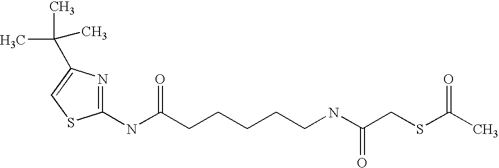 | Thioacetic acid S-{[5-(4-tert- butylthiazolylcarbamoyl)- pentylcarbamoyl]-methyl}ester | 1.49 | 386.06 |
| 46 | 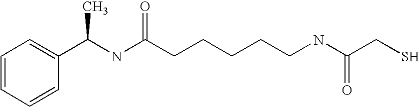 | 6-(2-Mercapto-acetylamino)- hexanoic acid (R-alpha- methylbenzyl)-amide | 1.18 | 309.17 |
| 47 | 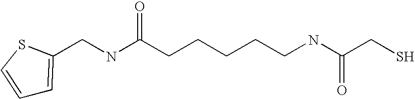 | 6-(2-Mercapto-acetylamino)- hexanoic acid (2-thiophenmethyl)- amide | 2.77 | 301.11 |
| 48 | 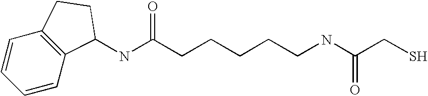 | 6-(2-Mercapto-acetylamino)- hexanoic acid indan-1-ylamide | 3.02 | 321.16 |
| 49 | 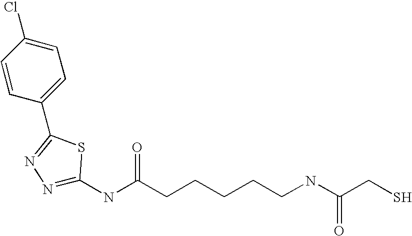 | 6-(2-Mercapto-acetylamino)- hexanoic acid [5-(4-chloro- phenyl)-[1,3,4]thiadiazol-2-yl]- amide | 3.31 | 399.2 |
| 50 | 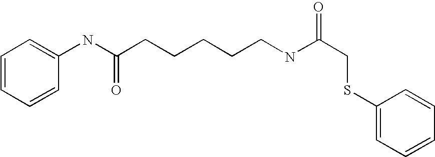 | 6-(2-Phenylsulfanyl-acetylamino)- hexanoic acid phenylamide | 3.29 | 357.16 |
| 51 | 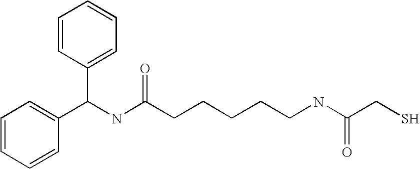 | 6-(2-Mercapto-acetylamino)- hexanoic acid diphenylmethylamide | 3.26 | 371.27 |
| 52 | 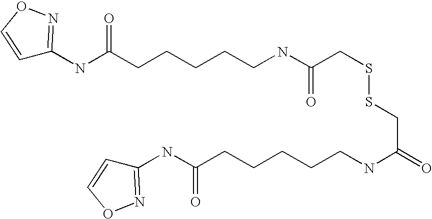 | 6-(2-{[5-(Isoxazol-3-ylcarbamoyl)- pentylcarbamoyl]- methyldisulfanyl}-acetylamino)- hexanoic acid isoxazol-3-ylamide | 2.82 | 541.34 |
| 53 | 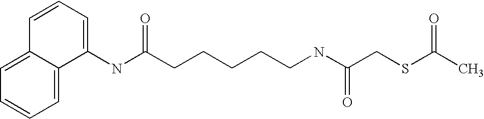 | Thioacetic acid S-{[5-(napht-1- ylcarbamoyl)-pentylcarbamoyl]- methyl}ester | 3.12 | 373.15 |
| 54 | 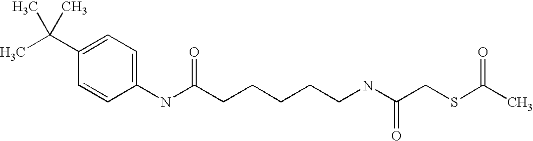 | Thioacetic acid S-{[5-(4-tert- butylphenylcarbamoyl)- pentylcarbamoyl]-methyl}ester | 1.56 | 379.08 |
| 55 | 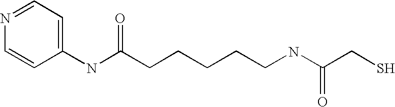 | 6-(2-Mercapto-acetylamino)- hexanoic acid pyridin-4-ylamide | 0.72 | 282.17 |
| 56 | 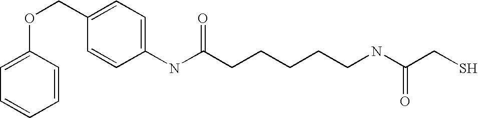 | 6-(2-Mercapto-acetylamino)- hexanoic acid (4-phenoxymethyl- phenyl)-amide | 3.71 | 278.16 observed, no MH⁺ |
| 57 | 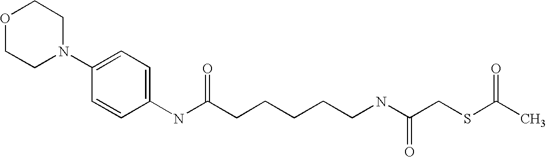 | Thioacetic acid S-{[5-(4- morpholin-4-ylphenylcarbamoyl)- pentylcarbamoyl]-methyl}ester | 1.09 | 408.06 |
| 58 | 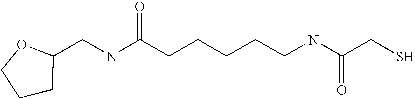 | 6-(2-Mercapto-acetylamino)- hexanoic acid (tetrahydro-furan-2- ylmethyl)-amide | 2.31 | 289.22 |
| 59 | 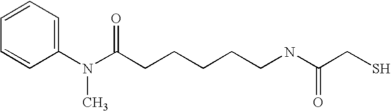 | 6-(2-Mercapto-acetylamino)- hexanoic acid methyl-phenyl- amide | 2.9 | 295.11 |
| 0 | 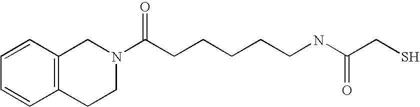 | N-[6-(3,4-Dihydro-1H-isoquinolin- 2-yl)-6-oxo-hexyl]-2-mercapto- acetamide | 3.04 | 321.18 |
| 61 | 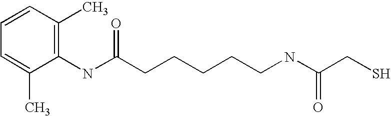 | 6-(2-Mercapto-acetylamino)- hexanoic acid (2,6-dimethyl- phenyl)-amide | 2.89 | 309.29 |
| 62 | 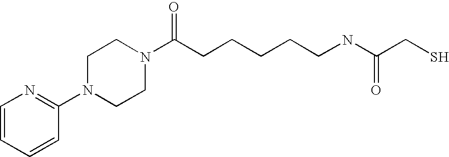 | 2-Mercapto-N-[6-oxo-6-(4- pyridin-2-yl-piperazin-1-yl)- hexyl]-acetamide | 2.27 | 350.21 |
| 63 | 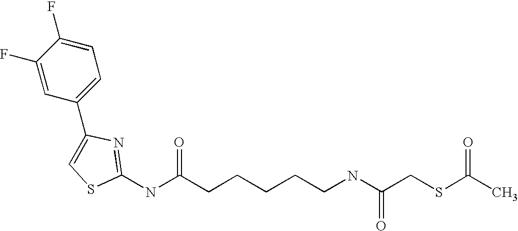 | Thioacetic acid S-({5-[4-(3,4- difluoro-phenyl)-thiazol-2- ylcarbamoyl]-pentylcarbamoyl}- methyl)ester | 1.59 | 441.97 |
| 64 | 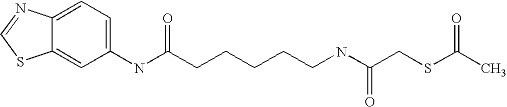 | Thioacetic acid S-{[5- (benzothiazol-6-ylcarbamoyl)- pentylcarbamoyl]-methyl}ester | 1.17 | 380.02 |
| 65 | 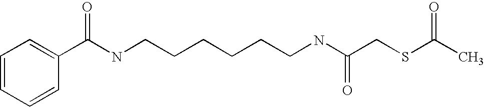 | Thioacetic acid S-[(6- benzoylamino-hexylcarbamoyl)- methyl]ester | 2.92 | 337.25 |
| 66 | 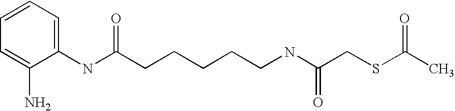 | Thioacetic acid S-{[5-(2-amino- phenylcarbamoyl)- pentylcarbamoyl]-methyl}ester | 0.86 | 338.14 |
| 67 | 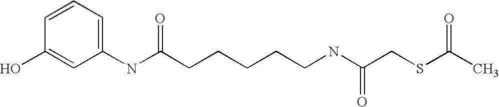 | Thioacetic acid S-{[5-(3-hydroxy- phenylcarbamoyl)- pentylcarbamoyl]-methyl}ester | 2.67 | 339.15 |
| 68 | 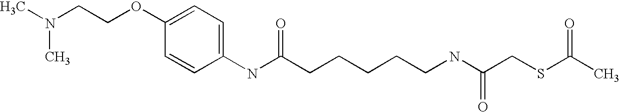 | Thioacetic acid S-({5-[4-(2- dimethylamino-ethoxy)- phenylcarbamoyl]- pentylcarbamoyl}-methyl) ester | 0.55 | 410.08 |
| 69 | 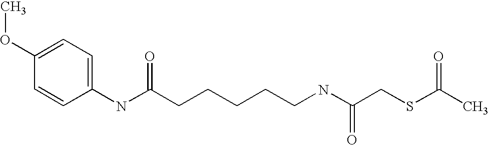 | Thioacetic acid S-{[5-(4-methoxy- phenylcarbamoyl)- pentylcarbamoyl]-methyl}ester | 2.9 | 353.15 |
| 70 | 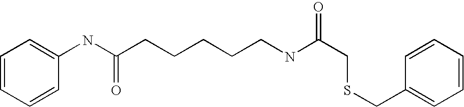 | 6-(2-Benzylsulfanyl-acetylamino)- hexanoic acid phenylamide | 3.42 | 371.15 |
| 71 | 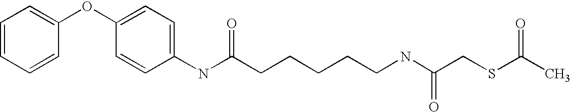 | Thioacetic acid S-{[5-(4-phenoxy- phenylcarbamoyl)- pentylcarbamoyl]-methyl}ester | 3.86 | 415.16 |
| 72 | 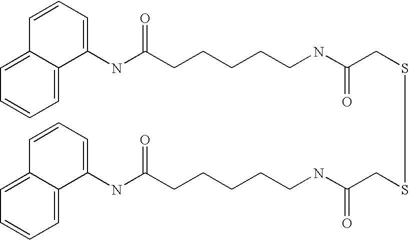 | 6-(2-{[5-(Naphthalen-1- ylcarbamoyl)-pentylcarbamoyl]- methyldisulfanyl}-acetylamino)- hexanoic acid naphthalen-1- ylamide | 3.49 | 660.28 |
| 73 | 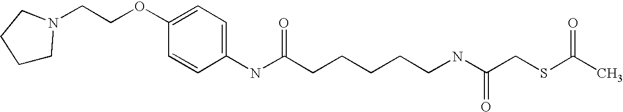 | Thioacetic acid S-({5-[4-(2- pyrrolidin-1-yl-ethoxy)- phenylcarbamoyl]- pentylcarbamoyl}-methyl) ester | 2.34 | 436.2 |
| 74 | 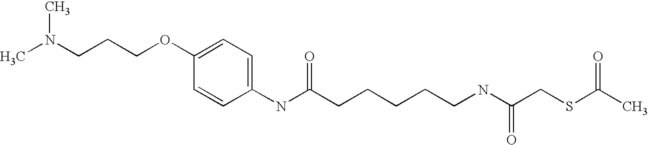 | Thioacetic acid S-({5-[4-(3- dimethylamino-propoxy)- phenylcarbamoyl]- entylcarbamoyl}-methyl) ester | 0.85 | 424.08 |
| 75 | 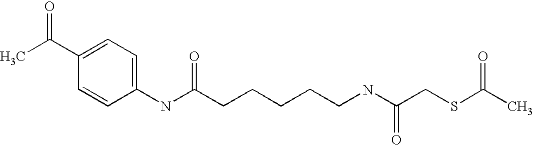 | Thioacetic acid S-{[5-(4-acetyl- phenylcarbamoyl)- pentylcarbamoyl]-methyl}ester | 1.21 | 365.09 |
| 76 | 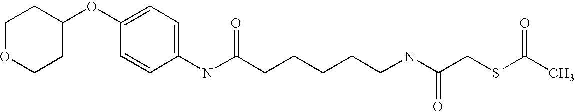 | Thioacetic acid S-({5-[4- (tetrahydro-pyran-4-yloxy)- phenylcarbamoyl]- pentylcarbamoyl}-methyl) ester | 2.92 | 423.18 |
| 77 | 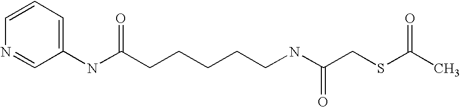 | Thioacetic acid S-{[5-(pyridin-3- ylcarbamoyl)-pentylcarbamoyl]- methyl}ester | 0.34 | 324.12 |
| 78 | 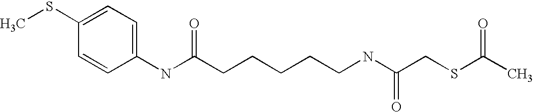 | Thioacetic acid S-{[5-(4- methylsulfanyl-phenylcarbamoyl)- pentylcarbamoyl]-methyl}ester | 1.34 | 369.07 |
| 79 | 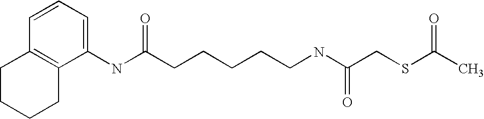 | Thioacetic acid S-{[5-(5,6,7,8- tetrahydro-naphthalen-1- ylcarbamoyl)-pentylcarbamoyl]- methyl}ester | 3.24 | 377.16 |
| 80 | 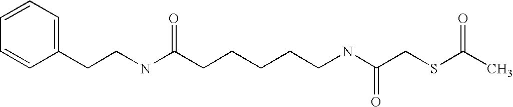 | Thioacetic acid S-{[5- phenethylcarbamoyl- pentylcarbamoyl]-methyl}ester | 2.97 | 351.14 |
| 81 | 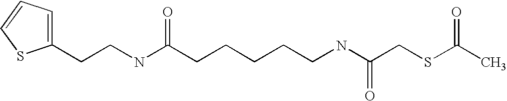 | Thioacetic acid S-{[5-(2-thiophen- 2-yl-ethylcarbamoyl)- pentylcarbamoyl]-methyl}ester | 1.21 | 357.08 |
| 82 | 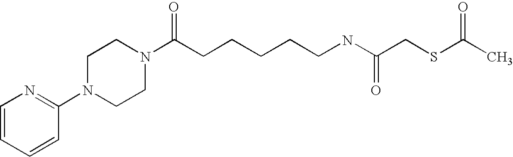 | Thioacetic acid S-{[6-oxo-6-(4- pyridin-2-yl-piperazin-1-yl)- hexylcarbamoyl]-methyl}ester | 0.63 | 393.11 |
| 83 | 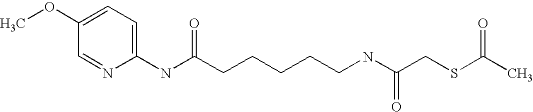 | Thioacetic acid S-{[5-(5-methoxy- pyridin-2-ylcarbamoyl)- pentylcarbamoyl]-methyl}ester | 2.72 | 354.12 |
| 84 | 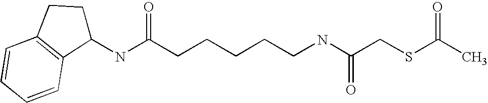 | Thioacetic acid S-{[5-(indan-1- ylcarbamoyl)-pentylcarbamoyl]- methyl}ester | 2.97 | 363.04 |
| 85 | 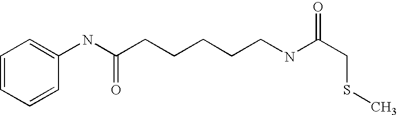 | 6-(2-Methylsulfanyl-acetylamino)- hexanoic acid phenylamide | 1.18 | 295.13 |
| 86 | 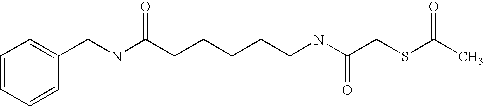 | Thioacetic acid S-[(5- benzylcarbamoyl- pentylcarbamoyl)-methyl]ester | 2.86 | 337.15 |
| 87 | 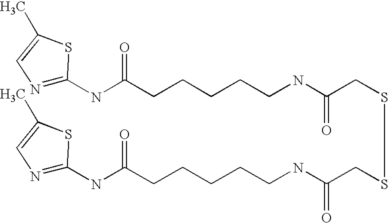 | 6-(2-{[5-(5-Methyl-thiazol-2- ylcarbamoyl)-pentylcarbamoyl]- methyldisulfanyl}-acetylamino)- hexanoic acid (5-methyl-thiazol-2- yl)-amide | 3.12 | 601.34 |
| 88 | 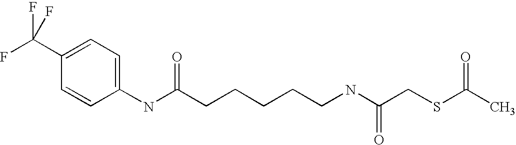 | Thioacetic acid S-{[5-(4- trifluorormethyl- phenylcarbamoyl)- pentylcarbamoyl]-methyl}ester | 1.49 | 391.04 |
| 89 | 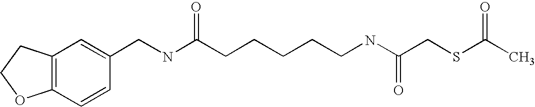 | Thioacetic acid S-({5-[(2,3- dihydro-benzofuran-5-ylmethyl)- carbamoyl]-pentylcarbamoyl}- methyl)ester | 2.87 | 379.26 |
| 90 | 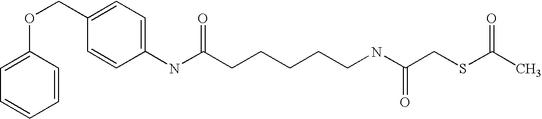 | Thioacetic acid S-{[5-(4- phenoxymethyl-phenylcarbamoyl)- pentylcarbamoyl]-methyl}ester | 1.53 | 429.07 |
| 91 | 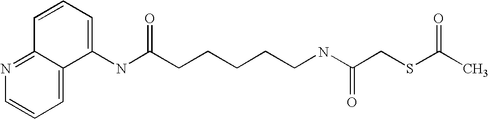 | Thioacetic acid S-{[5-quinolyn-5- ylarbamoyl-pentylcarbamoyl]- methyl}ester | 0.81 | 374.08 |
| 92 | 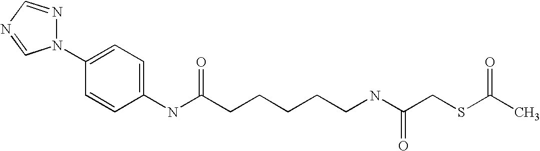 | Thioacetic acid S-{[5-(4- [1,2,4]triazol-1-yl- phenylcarbamoyl)- pentylcarbamoyl]-methyl}ester | 1.11 | 390.04 |
| 93 | 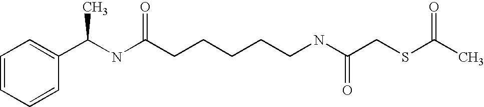 | Thioacetic acid S-{[5-(R-alpha- methylbenzyl)carbamoyl- pentylcarbamoyl]-methyl}ester | 2.95 | 351.14 |
| 94 | 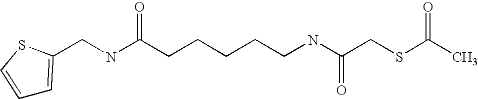 | Thioacetic acid S-{[5-[(thiophen-2- ylmethyl)carbamoyl]- pentylcarbamoyl]-methyl}ester | 2.82 | 343.1 |
| 95 | 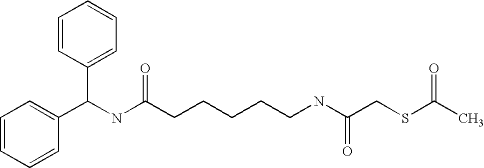 | Thioacetic acid S-{[5-(benzhydryl- carbamoyl)-pentylcarbamoyl]- methyl}ester | 3.36 | 413.16 |
| 96 | 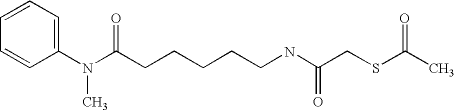 | Thioacetic acid S-{[5-(methyl- phenyl-carbamoyl)- pentylcarbamoyl]-methyl}ester | 3.02 | 337.13 |
| 97 | 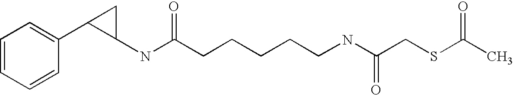 | Thioacetic acid S-{[5-(2-phenyl- cyclopropylcarbamoyl)- pentylcarbamoyl]-methyl}ester | 3.07 | 363.15 |
| 98 | 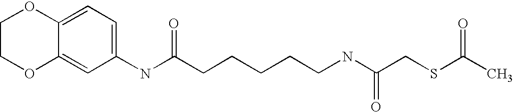 | Thioacetic acid S-{[5-(2,3- dihydro-benzo[1,4]dioxin-6- ylcarbamoyl)-pentylcarbamoyl]- methyl}ester | 2.94 | 381.16 |
| 99 | 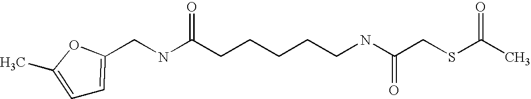 | Thioacetic acid S-({5-[(5-methyl- furan-2-ylmethyl)-carbamoyl]- pentylcarbamoyl}-methyl) ester | 2.84 | 341.26 |
| 100 | 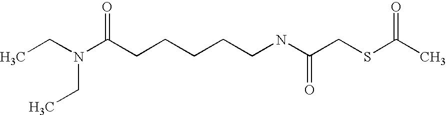 | Thioacetic acid S-[(5- diethylcarbamoyl- pentylcarbamoyl)-methyl]ester | 2.76 | 303.2 |
| 101 | 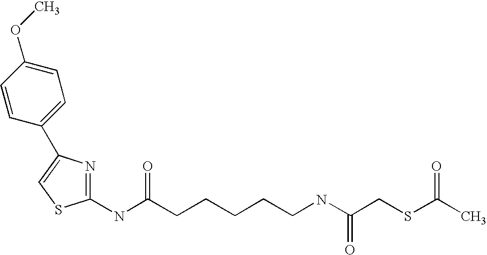 | Thioacetic acid S-({5-[4-(4- methoxy-phenyl)-thiazol-2- ylcarbamoyl]-pentylcarbamoyl}- methyl) ester | 3.34 | 436.22 |
| 102 | 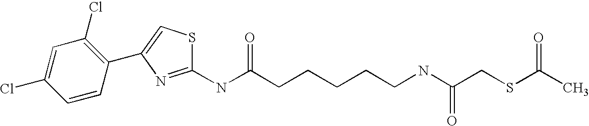 | Thioacetic acid S-({5-[4-(2,4- dichloro-phenyl)-thiazol-2- ylcarbamoyl]-pentylcarbamoyl}- methyl) ester | 3.76 | 474.23 |
| 103 | 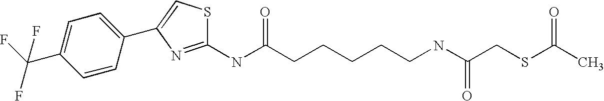 | Thioacetic acid S-({5-[4-(4- trifluoromethyl-phenyl)-thiazol-2- ylcarbamoyl]-pentylcarbamoyl}- methyl)ester | 3.76 | 474.29 |
| 104 | 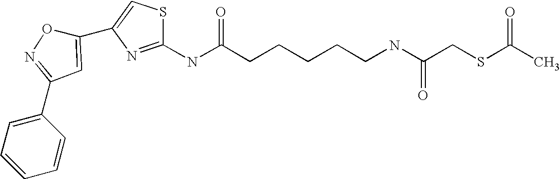 | Thioacetic acid S-({5-[4-(3- phenyl-isoxazol-5-yl)-thiazol-2- ylcarbamoyl]-pentylcarbamoyl}- methyl)ester | 3.54 | 473.32 |
| 105 | 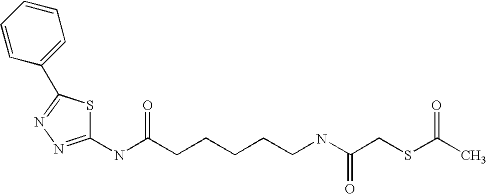 | Thioacetic acid S-{[5-(5-phenyl- [1,3,4]thiadiazol-2-ylcarbamoyl)- pentylcarbamoyl]-methyl}ester | 3.2 | 407.28 |
| 106 | 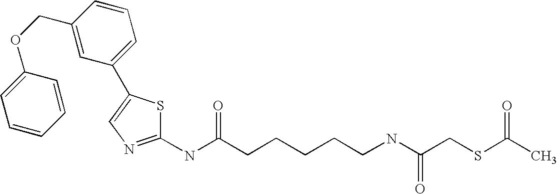 | Thioacetic acid S-({5-[5-(3- phenoxymethyl-phenyl)-thiazol-2- ylcarbamoyl]-pentylcarbamoyl}- methyl)ester | | |
| 107 | 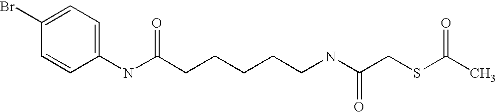 | Thioacetic acid S-{[5-(4-bromo- phenyl)carbamoyl- pentylcarbamoyl]-methyl}ester | 3.24 | 401.06 |
| 108 | 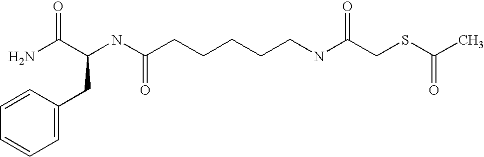 | Thioacetic acid S-{[5-(1- carbamoyl-2-S-phenyl- ethylcarbamoyl)- pentylcarbamoyl]-methyl}ester | 2.79 | 394.31 |
| 109 | 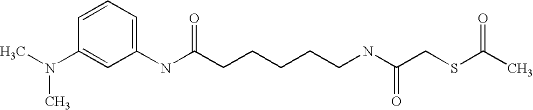 | Thioacetic acid S-{[5-(3- dimethylamino-phenylcarboamoyl)- pentylcarbamoyl]-methyl}ester | 2.26 | 366.31 |
| 110 | 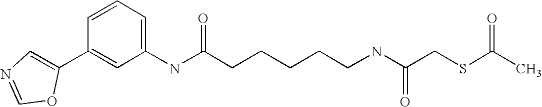 | 6-(2-Mercapto-acetylamino)- hexanoic acid (3-oxazol-5-yl- phenyl)amide | 2.86 | 348.29 |
| 111 | 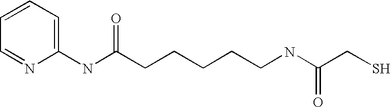 | 6-(2-Mercapto-acetylamino)- hexanoic acid pyridin-2-ylamide | 1.32 | 282.1 |
| 112 | 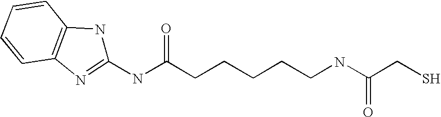 | 6-(2-Mercapto-acetylamino)- hexanoic acid (1H-benzoimidazol- 2-yl)-amide | 2.64 | 321.19 |
| 113 | 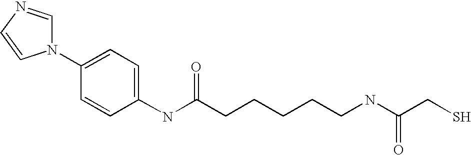 | 6-(2-Mercapto-acetylamino)- hexanoic acid (4-imidazol-1-yl- phenyl)amide | 2.44 | 346.23 |
| 114 | 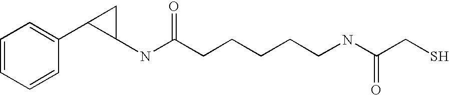 | 6-(2-Mercapto-acetylamino)- hexanoic acid (2-phenyl- cyclopropyl)-amide | 2.97 | 321.27 |
| 115 | 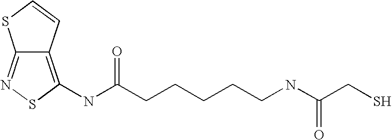 | 6-(2-Mercapto-acetylamino)- hexanoic acid thieno[2,3- c]isothiazol-3-ylamide | 2.77 | 313.14 observed, no MH⁺ |
| 116 | 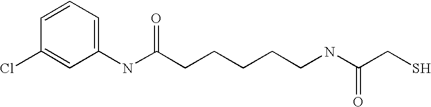 | 6-(2-Mercapto-acetylamino)- hexanoic acid (3-chloro- phenyl)amide | 3.19 | 315.21 |
| 117 | 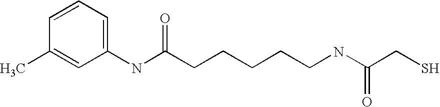 | 6-(2-Mercapto-acetylamino)- hexanoic acid m-tolylamide | 3.07 | 295.18 |
| 118 | 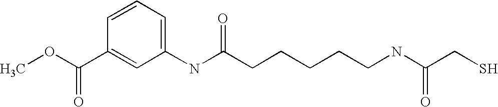 | 3-[6-(2-Mercapto-acetylamino)- hexanoylamino]-benzoic acid methyl ester | 2.86 | 339.25 |
| 119 | 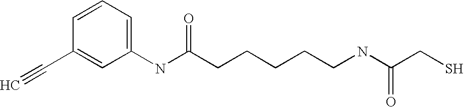 | 6-(2-Mercapto-acetylamino)- hexanoic acid (3-ethynyl- phenyl)amide | 3.09 | 305.21 |
| 120 | 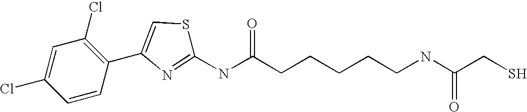 | 6-(2-Mercapto-acetylamino)- hexanoic acid [4-(2,4-dichloro- phenyl)-thiazol-2-yl]-amide | 3.74? | 414.29 |
| 121 | 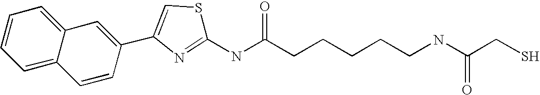 | 6-(2-Mercapto-acetylamino)- hexanoic acid (4-naphthalen-2-yl- thiazol-2-yl)-amide | 3.74 | 414.29 |
| 122 | 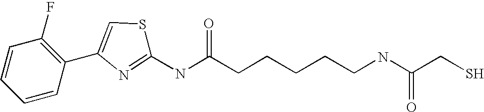 | 6-(2-Mercapto-acetylamino)- hexanoic acid [4-(2-fluoro- phenyl)-thiazol-2-yl]-amide | 3.61 | 382.18 |
| 123 | 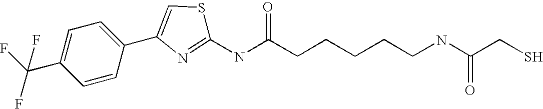 | 6-(2-Mercapto-acetylamino)- hexanoic acid [4-(4- trifluoromethyl-phenyl)-thiazol-2- yl]-amide | 3.67 | 432.28 |
| 124 | 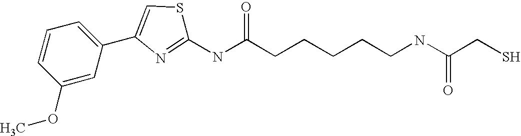 | 6-(2-Mercapto-acetylamino)- hexanoic acid [4-(3-methoxy- phenyl)-thiazol-2-yl]-amide | 3.31 | 394.19 |
| 125 | 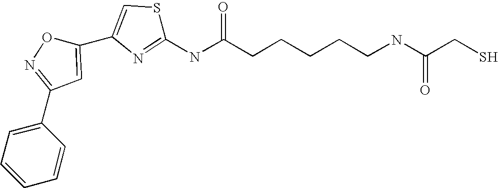 | 6-(2-Mercapto-acetylamino)- hexanoic acid [4-(3-phenyl- isoxazol-5-yl)-thiazol-2-yl]-amide | 3.56 | 431.29 |
| 126 | 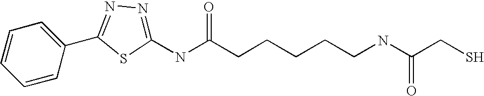 | 6-(2-Mercapto-acetylamino)- hexanoic acid (5-phenyl- [1,3,4]thiadiazol-2-yl)-amide | 3.06 | 365.26 |
| 127 | 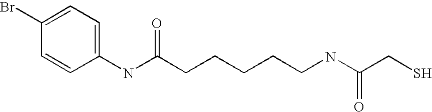 | 6-(2-Mercapto-acetylamino)- hexanoic acid (4-bromo- phenyl)amide | 3.18 | 361.18 |
| 128 | 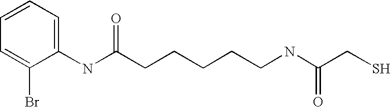 | 6-(2-Mercapto-acetylamino)- hexanoic acid (2-bromo- phenyl)amide | 2.87 | 361.17 |
| 129 | 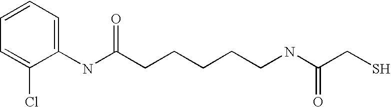 | 6-(2-Mercapto-acetylamino)- hexanoic acid (2-chloro- phenyl)amide | 2.88 | 315.21 |
| 130 | 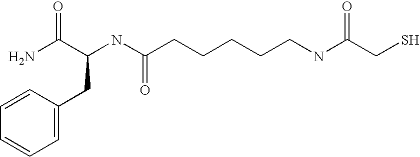 | 6-(2-Mercapto-acetylamino)- hexanoic acid (1-carbamoyl-2-S- phenyl-ethyl)-amide | 2.5 | 352.2 |
| 131 | 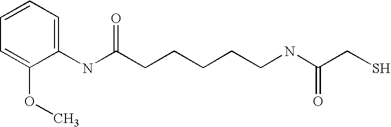 | 6-(2-Mercapto-acetylamino)- hexanoic acid (2-methoxy- phenyl)amide | 2.79 | 311.26 |
| 132 | 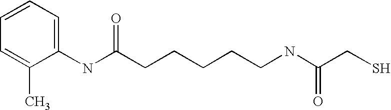 | 6-(2-Mercapto-acetylamino)- hexanoic acid o-tolylamide | 2.87 | 295.26 |
| 133 | 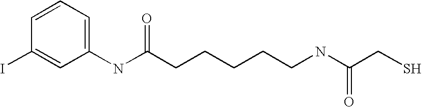 | 6-(2-Mercapto-acetylamino)- hexanoic acid (3-iodo- phenyl)amide | 3.2 | 407.18 |
| 134 | 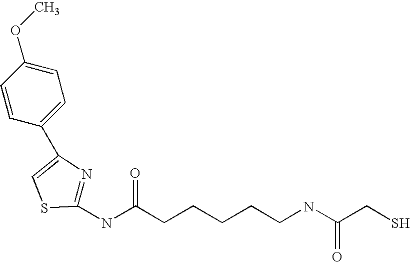 | 6-(2-Mercapto-acetylamino)- hexanoic acid [4-(4-methoxy- phenyl)-thiazol-2-yl]-amide | 3.31 | 394.28 |
| 135 | 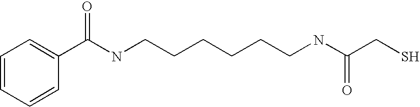 | N-[6-(2-Mercapto-acetylamino)- hexyl]-benzamide | 2.82 | 295.24 |
| 136 | 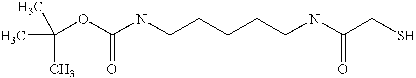 | [5-(2-Mercapto-acetylamino)- pentyl]-carbamic acid tert-butyl ester | 2.97 | 277.26 |
| 137 | 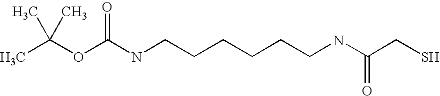 | [6-(2-Mercapto-acetylamino)- hexyl]-carbamic acid tert-butyl ester | 3.21 | 290.17 |
| 138 | 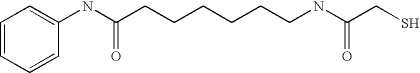 | 7-(2-Mercapto-acetylamino)- heptanoic acid phenylamide | 3.03 | 295.2 |
| 139 | 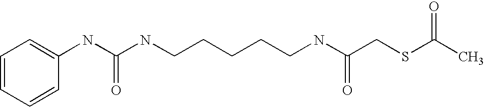 | Thioacetic acid S-{[5-(3-phenyl- ureido)-pentylcarbamoyl]-methyl}ester | 2.95 | 338.28 |
| 140 | 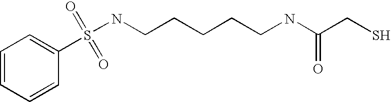 | N-(5-Benzenesulfonylamino- pentyl)-2-mercapto-acetamide | 2.95 | 317.23 |
| 141 | 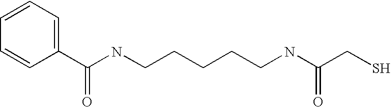 | N-[5 -(2-Mercapto-acetylamino)- pentyl]-benzamide | 2.62 | 281.16 |
| 142 | 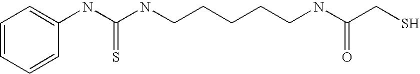 | 2-Mercapto-N-[5-(3-phenyl- thioureido)-pentyl]-acetamide | 2.81 | 312.15 |
| 143 | 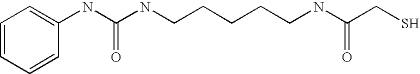 | 2-Mercapto-N-[5-(3-phenyl- ureido)-pentyl]-acetamide | 2.77 | 296.17 |
| 144 | 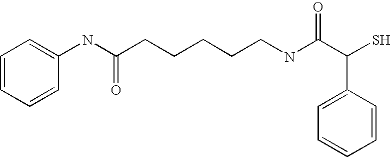 | 6-(2-Mercapto-2-phenyl- acetylamino)-hexanoic acid phenylamide | 3.38 | 357.1 |
| 145 | 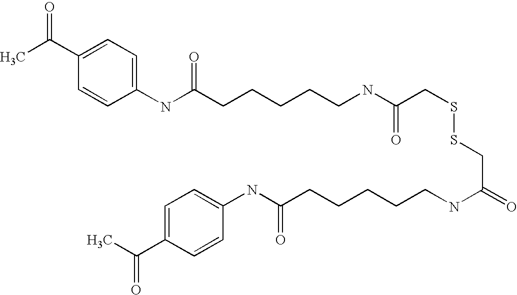 | 6-(2-{[5-(4-Acetyl- phenylcarbamoyl)- pentylcarbamoyl]- methyldisulfanyl}-acetylamino)- hexanoic acid (4-acetyl-phenyl)- amide | 3.11 | 644.5 |
| 146 | 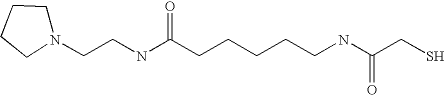 | 6-(2-Mercapto-acetylamino)- hexanoic acid (2-pyrrolidin-1-yl- ethyl)-amide | 0.21 | 301.24 |
| 147 | 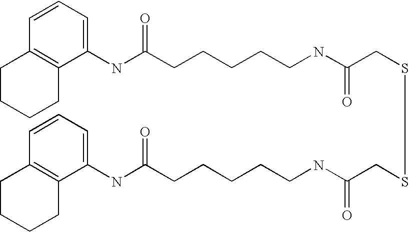 | 6-(2-{[5-(5,6,7,8-Tetrahydro- naphthalen-1-ylcarbamoyl)- pentylcarbamoyl]- methyldisulfanyl}-acetylamino)- hexanoic acid (5,6,7,8-tetrahydro- naphthalen-1-yl)-amide | 1.71 | 667.23 |
| 148 | 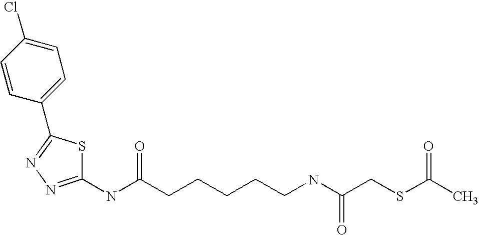 | Thioacetic acid S-({5-[5-(4-chloro- phenyl)-[1,3,4]thiadiazol-2-ylcarba moyl]-pentylcarbamoyl}-methyl) ester | 3.36 | 441.07 |
| 149 | 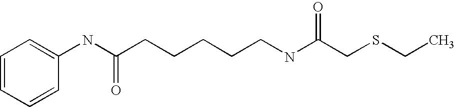 | 6-(2-Ethylsulfanyl-acetylamino)- hexanoic acid phenylamide | 3.02 | 309.26 |
| 150 | 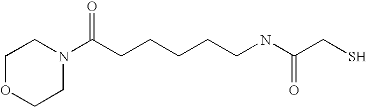 | 2-Mercapto-N-(6-morpholin-4-yl- 6-oxo-hexyl)-acetamide | 2.24 | 275.2 |
| 151 | 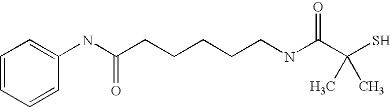 | 6-(2-Mercapto-2-methyl- propionylamino)-hexanoic acid phenylamide | 2.99 | 308.3 |
| 152 | 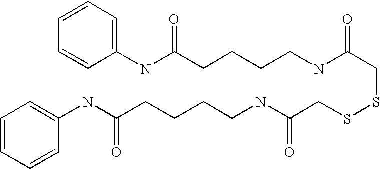 | 5-{2-[(4-Phenylcarbamoyl- butylcarbamoyl)- methyldisulfanyl]-acetylamino}- pentanoic acid phenylamide | 3.15 | 531.3 |
| 153 | 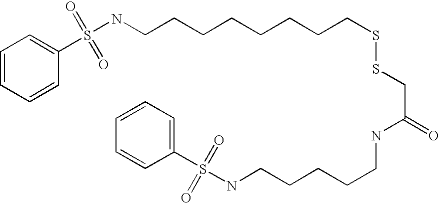 | N-(5-Benzenesulfonylamino- pentyl)-2-[(5- benzenesulfonylamino- pentylcarbamoyl)- methyldisulfanyl]-acetamide | 3.36 | 632.42 |
| 154 | 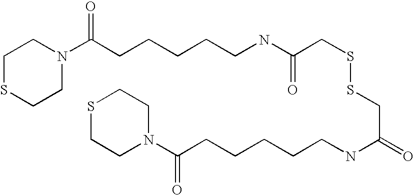 | N-(6-Oxo-6-thiomorpholin-4-yl- hexyl)-2-[(6-oxo-6-thiomorpholin- 4-yl-hexylcarbamoyl)- methyldisulfanyl]-acetamide | 3.05 | 579.41 |
| 155 | 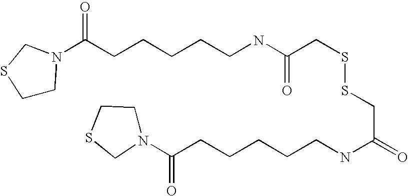 | N-(6-Oxo-6-thiazolidin-3-yl- hexyl)-2-[(6-oxo-6-thiazolidin-3- yl-hexylcarbamoyl)- methyldisulfanyl]-acetamide | 2.87 | 552.28 |

What is claimed is:

1. A compound represented by Formula IA: 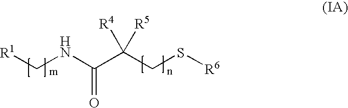
or a pharmaceutically acceptable salt thereof, wherein:
R¹ is R²NR³C(O)—, R²NHC(O)NH—, R²NHC(S)NH—, R²SO₂NH—, or R²C(O)NH—;
m is 4-6;
R² is a C₀₋₂alkyl, aryl, heteroaryl, carbocyclyl, -heteroaryl-heteroaryl, -heteroaryl-C₁₋₄alkyl, -heteroaryl-OCH₃, -heteroaryl-aryl-halogen, -heteroaryl-aryl, -aryl-aryl, -aryl-SCH₃, -aryl-OCH₃, -aryl-CF₃, -aryl-O—C₂alkyl-heterocyclyl, —C₃₋₁₀cycloalkyl-aryl, —C₀₋₂alkyl-heterocyclyl, —C₀₋₂alkyl-heteroaryl, —C₀₋₂alkyl-aryl, —C₀₋₁alkyl-heteroaryl, -aryl-OCH₂-aryl, -aryl-CH₂O-aryl, aryl-carbonyl, -aryl-carbonyl-aryl, -aryl-C(O)CH₃, aryl-O-aryl, -aryl-O-heterocyclyl, -aryl-C₁₋₄alkyl, -aryl-O—C₂₋₃alkyl-N(CH₃)(CH₃), C₀₋₁alkyl-heterocyclyl-C₀₋₁alkyl, C₀₋₁alkyl-heteroaryl-C₀₋₁alkyl, -heterocyclyl, -heterocyclyl-aryl, -heterocyclyl-heteroaryl, -aryl-heterocyclyl, -aryl-heteroaryl, or —CH(aryl)(aryl), any of which is optionally substituted with R²²;
R²² is C₀₋₄alkyl, halogen, —OH, —CF₃, —SCH₃, —OCH₃, —NH₂, —O(CH₂)₂N(CH₃)(CH₃), —OCH₂-aryl, —O(CH₂)₂-heterocyclyl, —C(O)CH₃, —O-heterocyclyl, aryloxy-C₀₋₁alkyl-, aryl, or heterocyclyl;
R³ is C₀₋₁alkyl, or R² and R³ taken together form a heterocyclic or carbocyclic ring, any of which is optionally substituted with one or more independent C₁₋₄alkyl, halogen, —OH, —SCH₃, —OCH₃, —NH₂, aryl, or heterocyclyl substituents;
R⁴ and R⁵ are each independently C₀₋₄alkyl, phenyl, or fluorine;
n is 0 or 1; and
R⁶ is hydrogen, methyl, ethyl, phenyl, benzyl, or acetyl.

2. The compound of claim 1 wherein R¹ is R²NR³C(O)—.

3. The compound of claim 2 wherein R² is —C₀₋₂alkyl-aryl which is optionally substituted with R²².

4. The compound of claim 2 wherein R² is —C₀₋₂alkyl-heteroaryl which is optionally substituted with R²².

5. The compound of claim 2 wherein R² is —C₀₋₂alkyl-heterocyclyl which is optionally substituted with R²².

6. The compound of claim 2 wherein R² is a carbocyclyl which is optionally substituted with R²².

7. The compound of claim 2 wherein R² is —CH(aryl)(aryl) which is optionally substituted with R²².

8. The compound of claim 1 wherein R¹ is R²NR³C(O)—, and R² and R³ are taken together to form a ring wherein said ring is optionally substituted with R²².

9. The compound of claim 8 wherein R² and R³ are taken together to form a heterocyclic ring wherein said ring is optionally substituted with R²².

10. The compound of claim 8 wherein R² and R³ are taken together to form a carbocyclic ring wherein said ring is optionally substituted with R²².

11. The compound of claim 1 wherein R¹ is R²NHC(O)NH—.

12. The compound of claim 1 wherein R¹ is R²NHC(S)NH—.

13. The compound of claim 1 wherein R¹ is R²SO₂NH—.

14. The compound of claim 1 wherein R¹ is R₂C(O)NH—.

15. A compound represented by Formula IB: 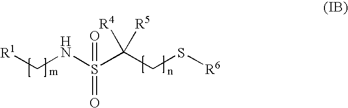
or a pharmaceutically acceptable salt thereof, wherein:
R¹ is R²NR³C(O)—, R²NHC(O)NH—, R²NHC(S)NH—, R²SO₂NH—, or R²C(O)NH—;
m is 4-6;
R² is a C₀₋₂alkyl, aryl, heteroaryl, carbocyclyl, -heteroaryl-heteroaryl, -heteroaryl-C₁₋₄alkyl, -heteroaryl-OCH₃, -heteroaryl-aryl-halogen, -heteroaryl-aryl, -aryl-aryl, -aryl-SCH₃, -aryl-OCH₃, -aryl-CF₃, -aryl-O—C₂alkyl-heterocyclyl, —C₃₋₁₀cycloalkyl-aryl, —C₀₋₂alkyl-heterocyclyl, —C₀₋₂alkyl-heteroaryl, —C₀₋₂alkyl-aryl, —C₀₋₁alkyl-heteroaryl, -aryl-OCH₂-aryl, -aryl-CH₂O-aryl, aryl-carbonyl, -aryl-carbonyl-aryl, -aryl-C(O)CH₃, aryl-O-aryl, -aryl-O-heterocyclyl, -aryl-C₁₋₄alkyl, -aryl-O—C₂₋₃alkyl-N(CH₃)(CH₃), C₀₋₁alkyl-heterocyclyl-C₀₋₁alkyl, C₀₋₁alkyl-heteroaryl-C₀₋₁alkyl, -heterocyclyl, -heterocyclyl-aryl, -heterocyclyl-heteroaryl, -aryl-heterocyclyl, -aryl-heteroaryl, or —CH(aryl)(aryl), any of which is optionally substituted with R²²;
R²² is C₀₋₄alkyl, halogen, —OH, —CF₃, —SCH₃, —OCH₃, —NH₂, —O(CH₂)₂N(CH₃)(CH₃), —OCH₂-aryl, —O(CH₂)₂-heterocyclyl, —C(O)CH₃, —O-heterocyclyl, aryloxy-C₀₋₁alkyl-, aryl, or heterocyclyl;
R³ is C₀₋₁alkyl, or R² and R³ taken together form a heterocyclic or carbocyclic ring, any of which is optionally substituted with one or more independent C₁₋₄alkyl, halogen, —OH, —SCH₃, —OCH₃, —NH₂, aryl, or heterocyclyl substituents;
R⁴ and R⁵ are each independently C₀₋₄alkyl, phenyl, or fluorine;
n is 0 or 1; and
R⁶ is hydrogen, methyl, ethyl, phenyl, benzyl, or acetyl.

16. A compound represented by Formula IIA: 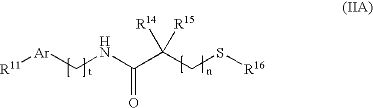
or a pharmaceutically acceptable salt thereof, wherein:
Ar is aryl optionally substituted with one or more independent C₁₋₄alkyl, halogen, —OH, —SCH₃, —OCH₃, —NH₂, aryl, or heterocyclyl substituents;
R¹¹ is R¹²NR¹³C(O)(CH₂)₁₋₂—, R¹²NHC(O)NH(CH₂)₁₋₂—, R¹²NHC(S)NH(CH₂)₁₋₂—, R¹²SO₂NH(CH₂)₁₋₂—, or R¹²C(O)NH(CH₂)₁₋₂—;
t is 1 or 2;
R¹² is a C₀₋₂alkyl, aryl, heteroaryl, carbocyclyl, -heteroaryl-heteroaryl, -heteroaryl-C₁₋₄alkyl, -heteroaryl-OCH₃, -heteroaryl-aryl-halogen, -heteroaryl-aryl, -aryl-aryl, -aryl-SCH₃, -aryl-OCH₃, -aryl-CF₃, -aryl-O—C₂alkyl-heterocyclyl, —C₃₋₁₀cycloalkyl-aryl, —C₀₋₂alkyl-heterocyclyl, —C₀₋₂alkyl-heteroaryl, —C₀₋₂alkyl-aryl, —C₀₋₁alkyl-heteroaryl, -aryl-OCH₂-aryl, -aryl-CH₂O-aryl, aryl-carbonyl, -aryl-carbonyl-aryl, -aryl-C(O)CH₃, aryl-O-aryl, -aryl-O-heterocyclyl, -aryl-C₁₋₄alkyl, -aryl-O—C₂₋₃alkyl-N(CH₃)(CH₃), C₀₋₁alkyl-heterocyclyl-C₀₋₁alkyl, C₀₋₁alkyl-heteroaryl-C₀₋₁alkyl, -heterocyclyl, -heterocyclyl-aryl, -heterocyclyl-heteroaryl, -aryl-heterocyclyl, -aryl-heteroaryl, or —CH(aryl)(aryl), any of which is optionally substituted with R²²²;
R²²² is C₀₋₄alkyl, halogen, —OH, —CF₃, —SCH₃, —OCH₃, —NH₂, —O(CH₂)₂N(CH₃)(CH₃), —OCH₂-aryl, —O(CH₂)₂-heterocyclyl, —C(O)CH₃, —O-heterocyclyl, aryloxy-C₀₋₁alkyl-, aryl, or heterocyclyl;
R¹³ is C₀₋₁alkyl, or R¹² and R¹³ taken together form a heterocyclic or carbocyclic ring, any of which is optionally substituted with one or more independent C₁₋₄alkyl, halogen, —OH, —SCH₃, —OCH₃, —NH₂, aryl, or heterocyclyl substituents;
R¹⁴ and R¹⁵ are each independently C₀₋₄alkyl, phenyl, or fluorine;
n is 0 or 1; and
R¹⁶ is hydrogen, methyl, ethyl, phenyl, benzyl, or acetyl.

17. A compound represented by Formula IIB: 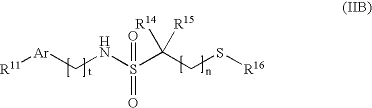
or a pharmaceutically acceptable salt thereof, wherein:
R¹¹ is R¹²NR¹³C(O)(CH₂)₁₋₂—, R¹²NHC(O)NH(CH₂)₁₋₂—, R¹²NHC(S)NH(CH₂)₁₋₂, R¹²SO₂NH(CH₂)₁₋₂—, or R¹²C(O)NH(CH₂)₁₋₂—;
t is 1 or 2;
R¹² is a C₀₋₂alkyl, aryl, heteroaryl, carbocyclyl, -heteroaryl-heteroaryl, -heteroaryl-C₁₋₄alkyl, -heteroaryl-OCH₃, -heteroaryl-aryl-halogen, -heteroaryl-aryl, -aryl-aryl, -aryl-SCH₃, -aryl-OCH₃, -aryl-CF₃, -aryl-O—C₂alkyl-heterocyclyl, —C₃₋₁₀cycloalkyl-aryl, —C₀₋₂alkyl-heterocyclyl, —C₀₋₂alkyl-heteroaryl, —C₀₋₂alkyl-aryl, —C₀₋₁alkyl-heteroaryl, -aryl-OCH₂-aryl, -aryl-CH₂O-aryl, aryl-carbonyl, -aryl-carbonyl-aryl, -aryl-C(O)CH₃, aryl-O-aryl, -aryl-O-heterocyclyl, -aryl-C₁₋₄alkyl, -aryl-O—C₂₋₃alkyl-N(CH₃)(CH₃), C₀₋₁alkyl-heterocyclyl-C₀₋₁alkyl, C₀₋₁alkyl-heteroaryl-C₀₋₁alkyl, -heterocyclyl, -heterocyclyl-aryl, -heterocyclyl-heteroaryl, -aryl-heterocyclyl, -aryl-heteroaryl, or —CH(aryl)(aryl), any of which is optionally substituted with R²²²;
R²²² is C₁₋₄alkyl, halogen, —OH, —CF₃, —SCH₃, —OCH₃, —NH₂, —O(CH₂)₂N(CH₃)(CH₃), —OCH₂-aryl, —O(CH₂)₂-heterocyclyl, —C(O)CH₃, —O-heterocyclyl, aryloxy-C₀₋₁alkyl-, aryl, or heterocyclyl;
R¹³ is C₀₋₁alkyl, or R¹² and R¹³ taken together form a heterocyclic or carbocyclic ring, any of which is optionally substituted with one or more independent C₁₋₄alkyl, halogen, —OH, —SCH₃, —OCH₃, —NH₂, aryl, or heterocyclyl substituents;
R¹⁴ and R¹⁵ are each independently C₀₋₄alkyl, phenyl, or fluorine;
n is 0 or 1; and
R¹⁶ is hydrogen, methyl, ethyl, phenyl, benzyl, or acetyl.

18. The compound of claim 1, or a pharmaceutically acceptable salt thereof, wherein a homo-dimer of the compound is present at R⁶.

19. The compound of claim 15, or a pharmaceutically acceptable salt thereof, wherein a homo-dimer of the compound is present at R⁶.

20. The compound of claim 16, or a pharmaceutically acceptable salt thereof, wherein a homo-dimer of the compound is present at R¹⁶.

21. The compound of claim 17, or a pharmaceutically acceptable salt thereof, wherein a homo-dimer of the compound is present at R¹⁶.

22. A compound selected from the group consisting of:

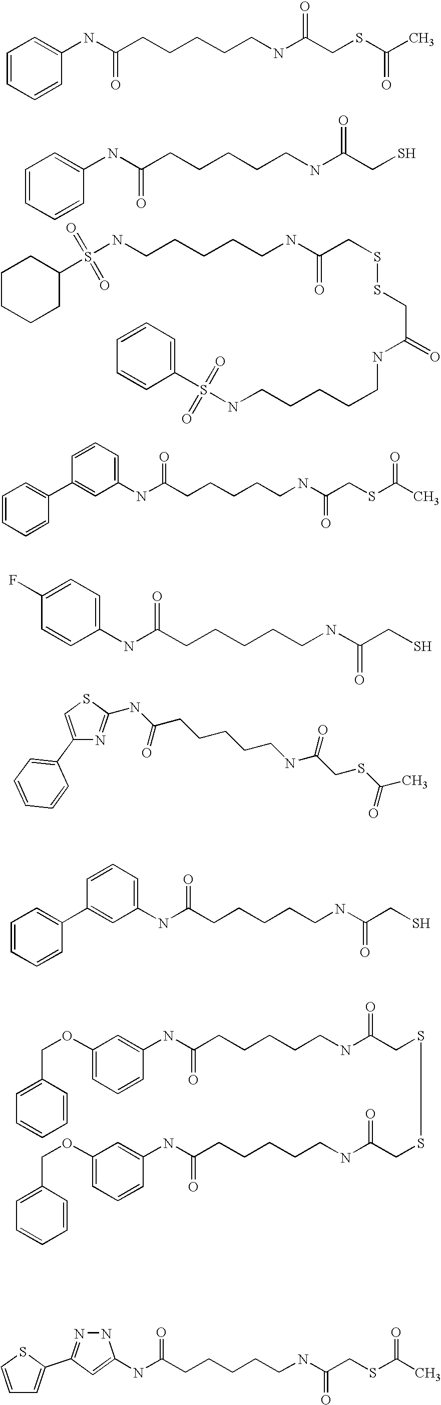

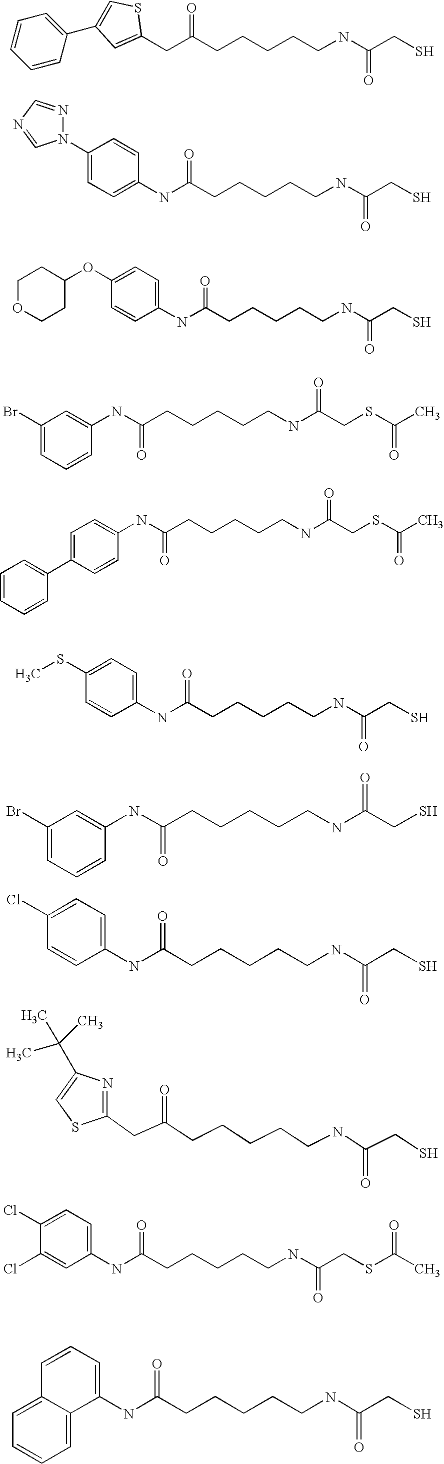

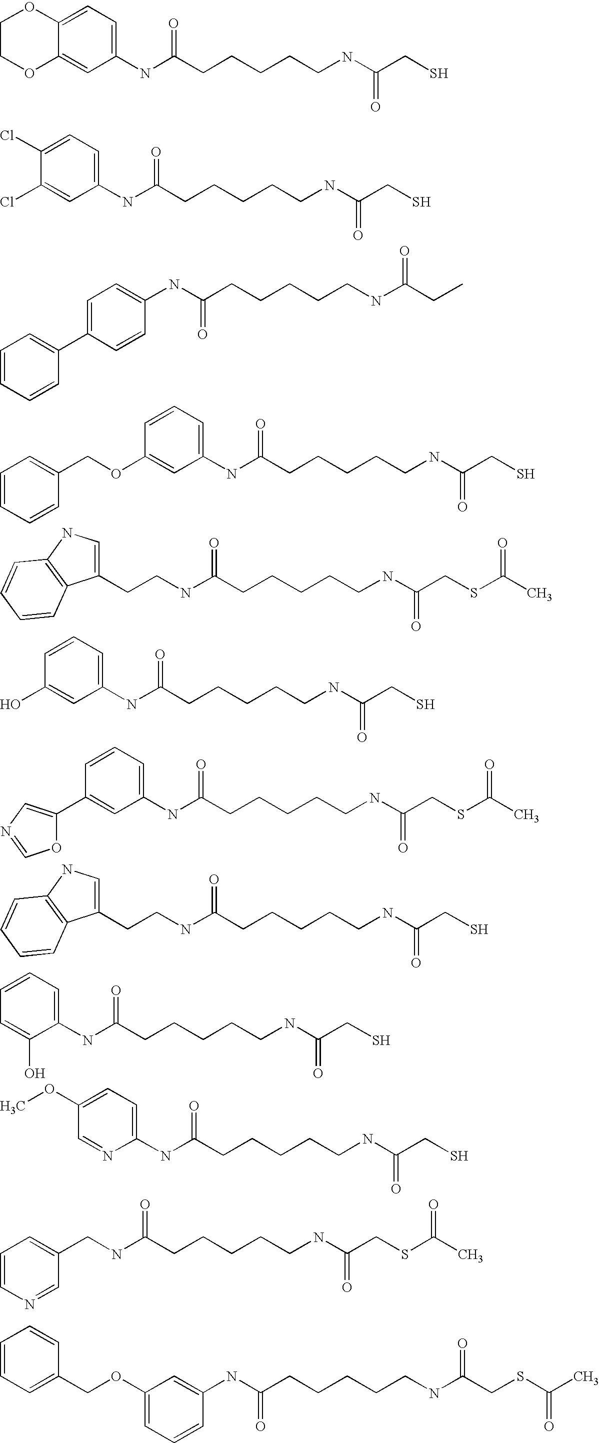

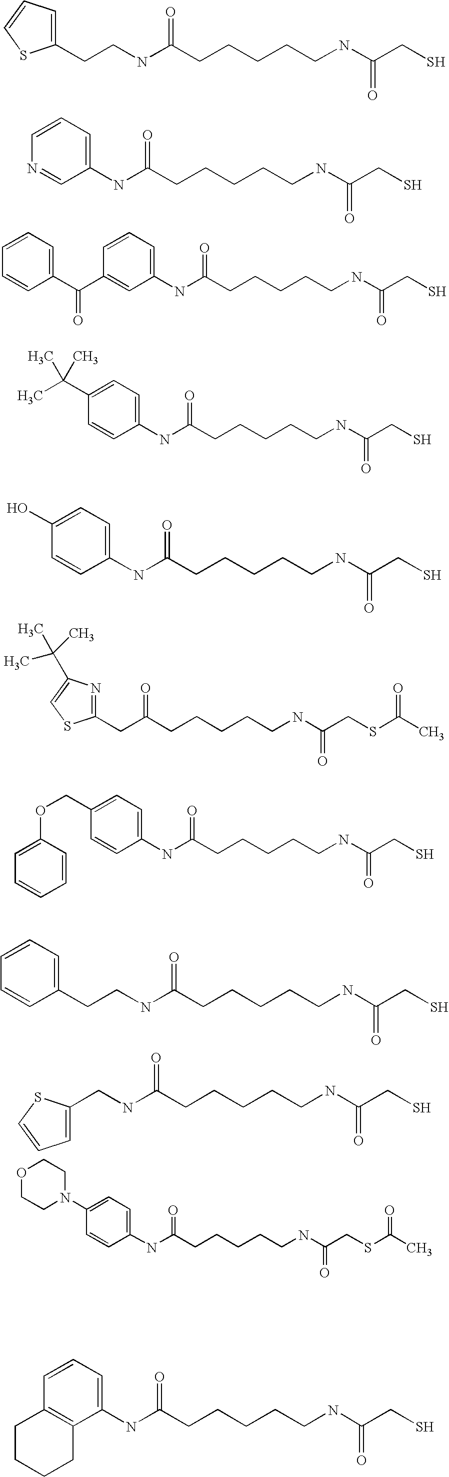

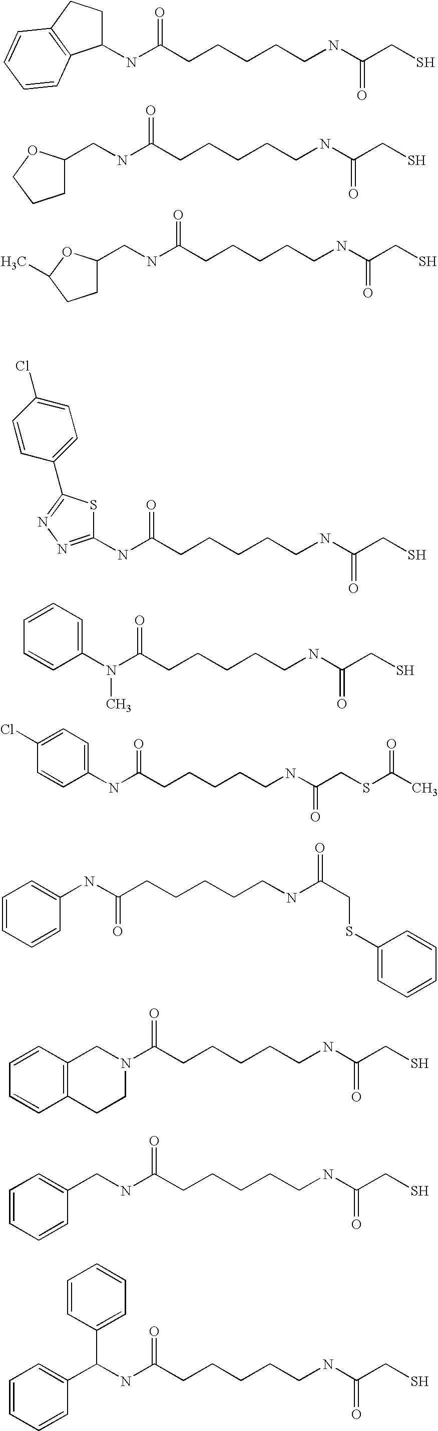

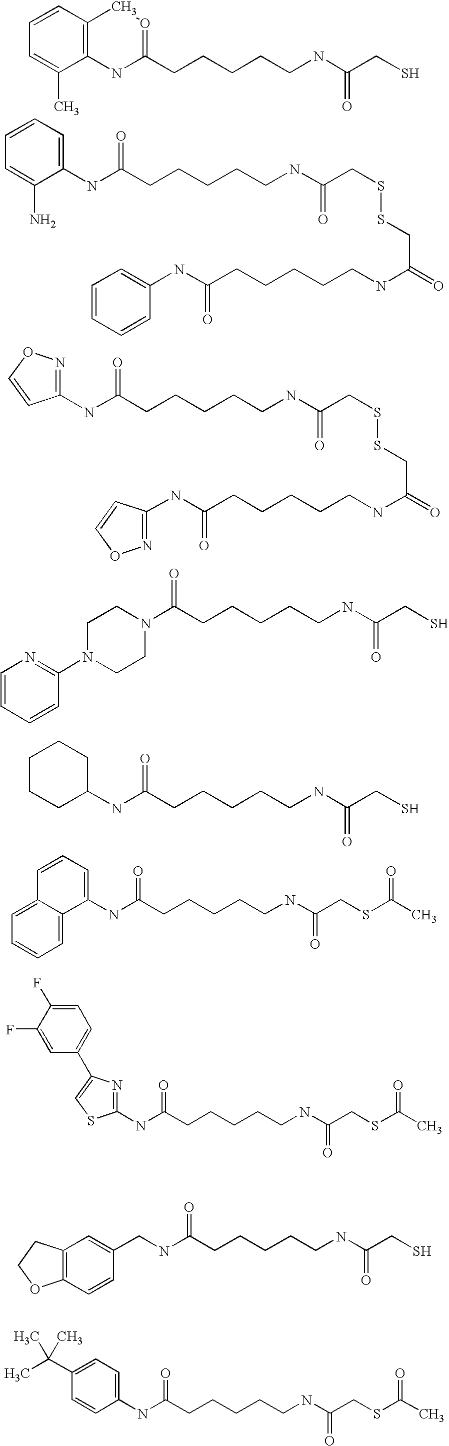

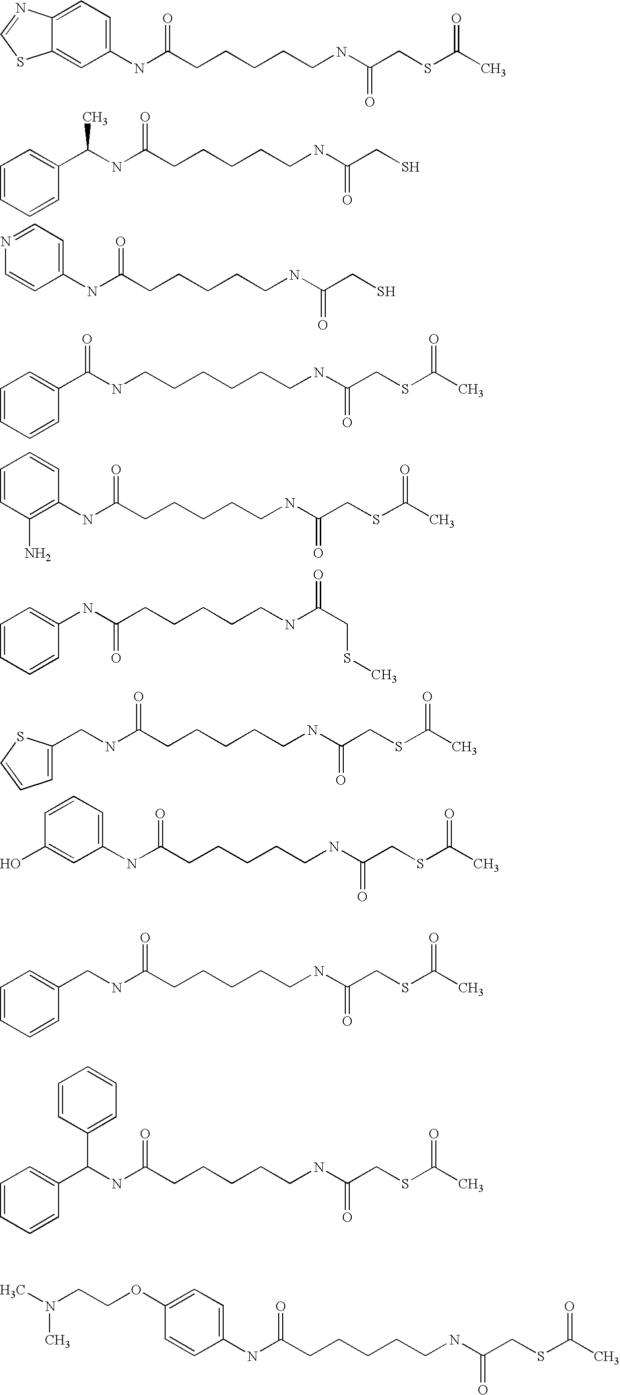

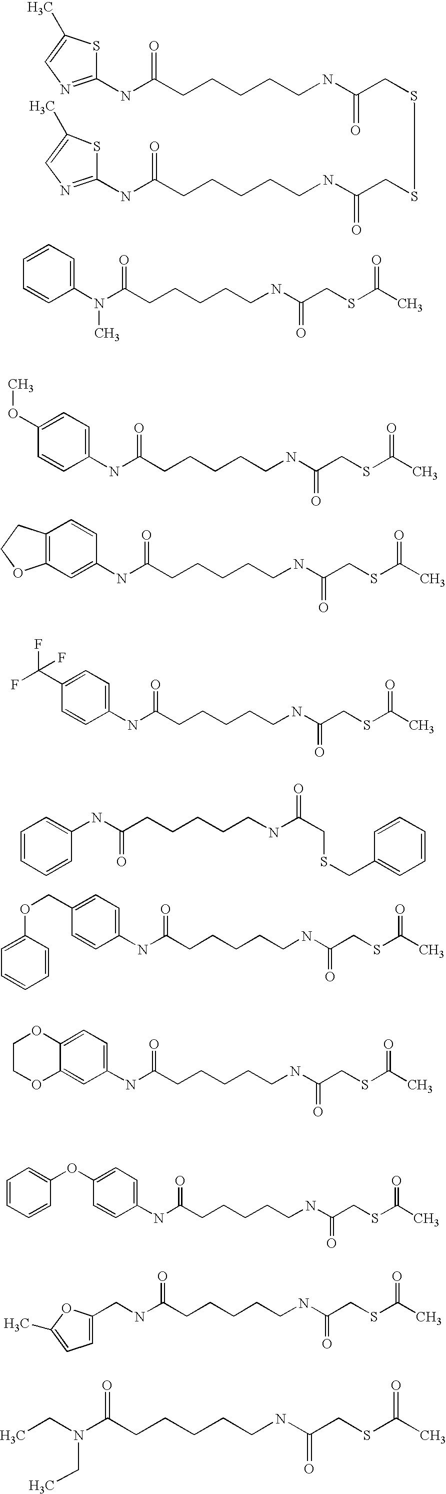

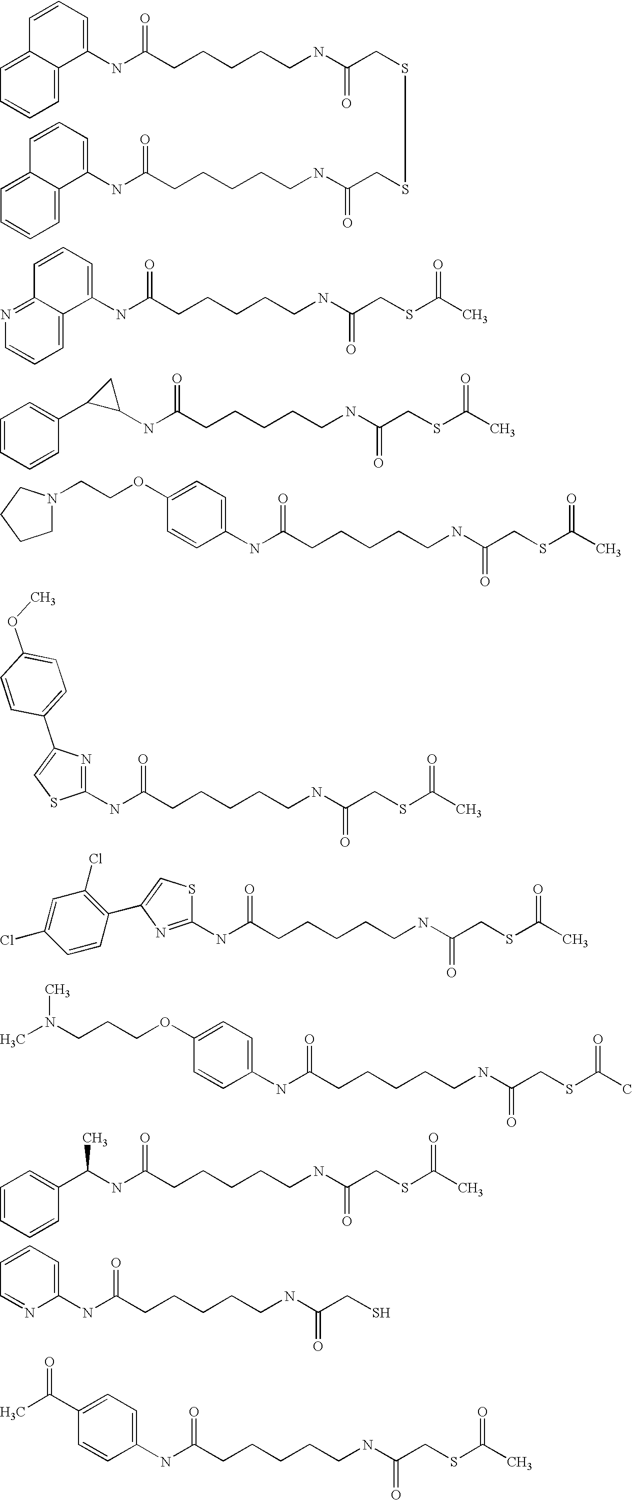

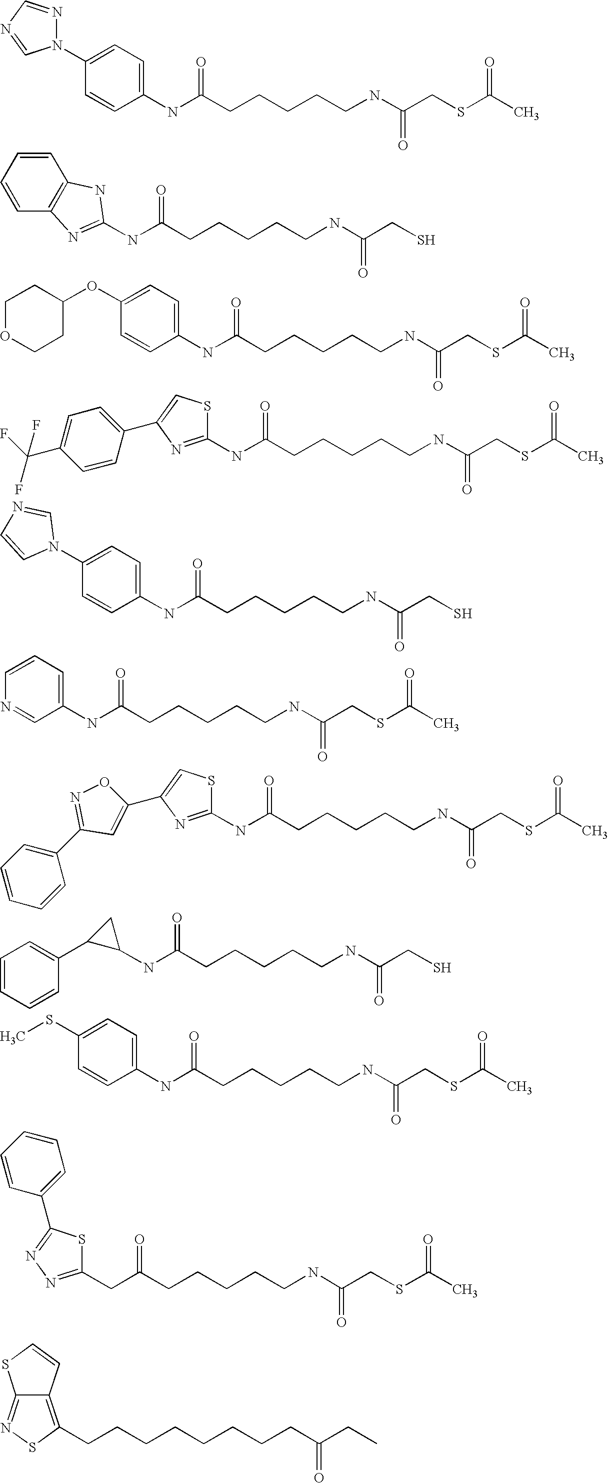

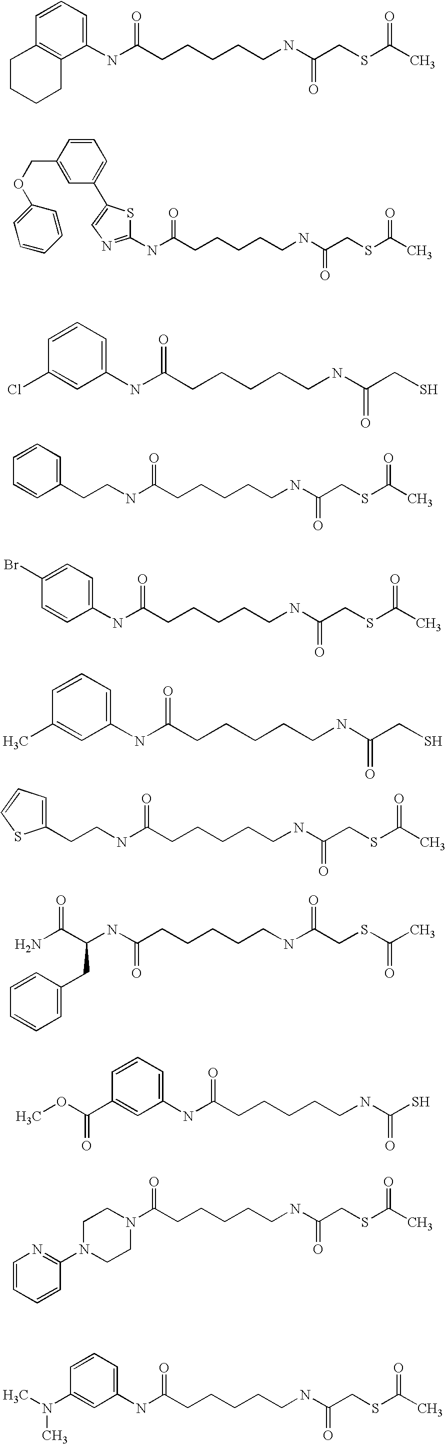

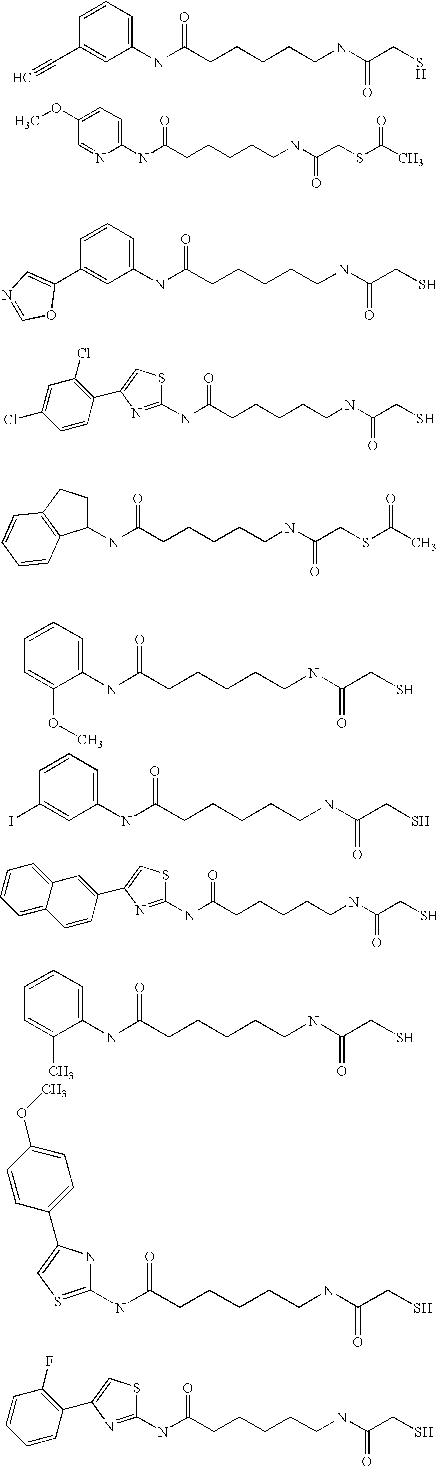

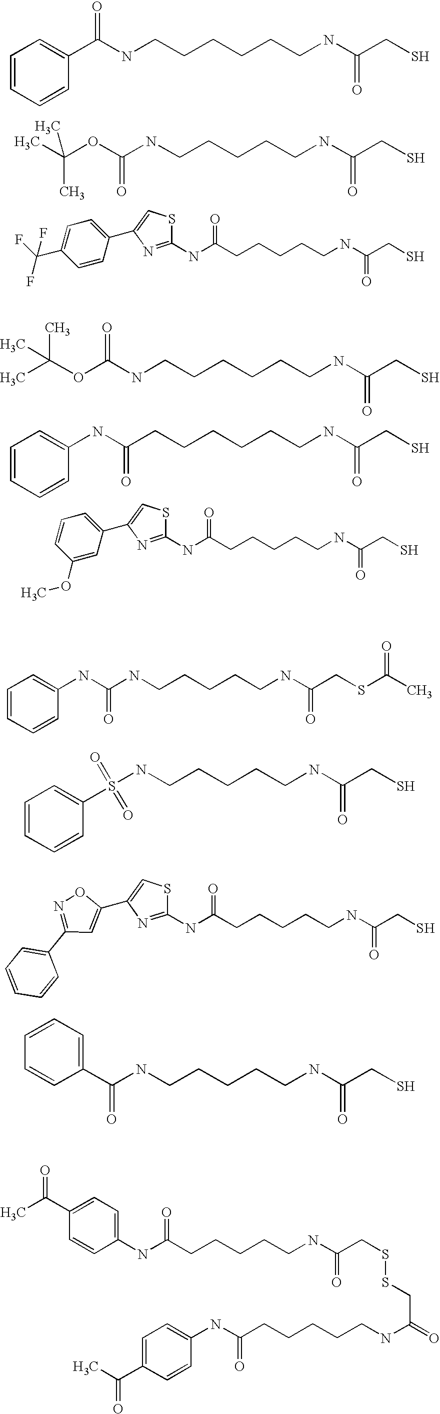

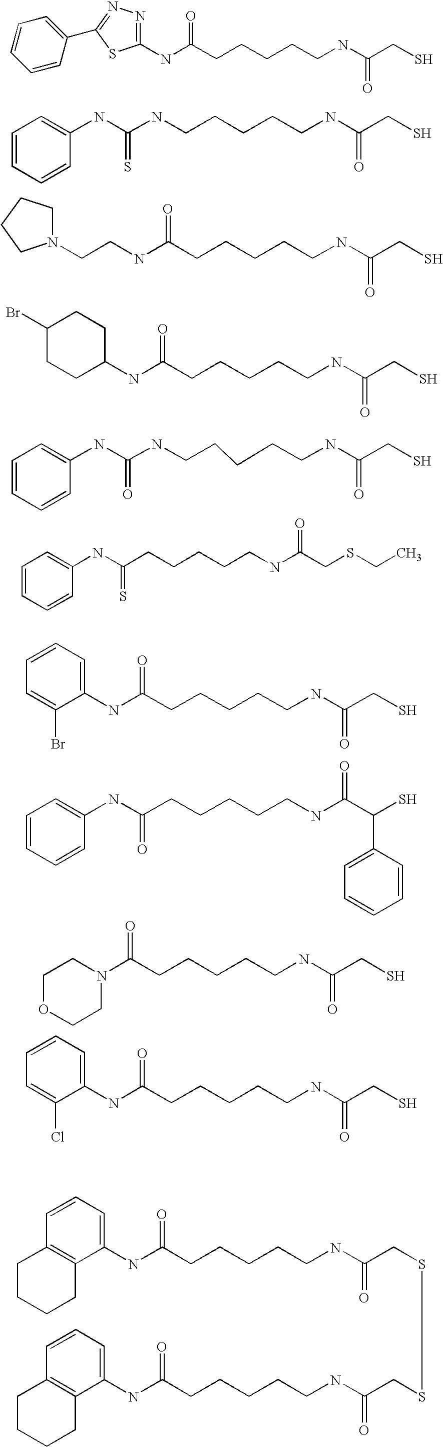

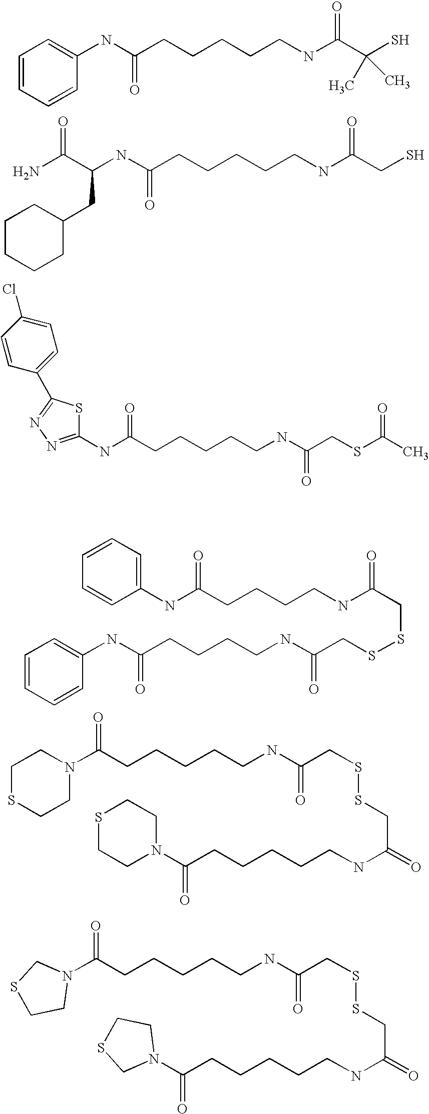

23. A pharmaceutical composition comprising a therapeutically effective amount of a compound of claim 1, or a pharmaceutically acceptable salt thereof, and a pharmaceutically acceptable carrier.

24. A pharmaceutical composition comprising a therapeutically effective amount of a compound of claim 15, or a pharmaceutically acceptable salt thereof, and a pharmaceutically acceptable carrier.

25. A pharmaceutical composition comprising a therapeutically effective amount of a compound of claim 16, or a pharmaceutically acceptable salt thereof, and a pharmaceutically acceptable carrier.

26. A pharmaceutical composition comprising a therapeutically effective amount of a compound of claim 17, or a pharmaceutically acceptable salt thereof, and a pharmaceutically acceptable carrier.

27. A pharmaceutical composition comprising a therapeutically effective amount of a compound of claim 18, or a pharmaceutically acceptable salt thereof, and a pharmaceutically acceptable carrier.

28. A pharmaceutical composition comprising a therapeutically effective amount of a compound of claim 19, or a pharmaceutically acceptable salt thereof, and a pharmaceutically acceptable carrier.

29. A pharmaceutical composition comprising a therapeutically effective amount of a compound of claim 20, or a pharmaceutically acceptable salt thereof, and a pharmaceutically acceptable carrier.

30. A pharmaceutical composition comprising a therapeutically effective amount of a compound of claim 21, or a pharmaceutically acceptable salt thereof, and a pharmaceutically acceptable carrier.

31. A method for treating cancerous diseases, infections, or metabolic disorders in a mammal by inhibiting histone deacetylase enzyme comprising administrating to said mammal a therapeutically effective amount of the compound according to claim 1, or a pharmaceutically acceptable salt thereof.

32. A method for treating cancerous diseases, infections, or metabolic disorders in a mammal by inhibiting histone deacetylase enzyme comprising administrating to said mammal a therapeutically effective amount of the compound according to claim 15, or a pharmaceutically acceptable salt thereof.

33. A method for treating cancerous diseases, infections, or metabolic disorders in a mammal by inhibiting histone deacetylase enzyme comprising administrating to said mammal a therapeutically effective amount of the compound according to claim 16, or a pharmaceutically acceptable salt thereof.

34. A method for treating cancerous diseases, infections, or metabolic disorders in a mammal by inhibiting histone deacetylase enzyme comprising administrating to said mammal a therapeutically effective amount of the compound according to claim 17, or a pharmaceutically acceptable salt thereof.

35. A method for treating cancerous diseases, infections, or metabolic disorders in a mammal by inhibiting histone deacetylase enzyme comprising administrating to said mammal a therapeutically effective amount of the pharmaceutical composition according claim 23.

36. A method for treating cancerous diseases, infections, or metabolic disorders in a mammal by inhibiting histone deacetylase enzyme comprising administrating to said mammal a therapeutically effective amount of the pharmaceutical composition according to claim 24.

37. A method for treating cancerous diseases, infections, or metabolic disorders in a mammal by inhibiting histone deacetylase enzyme comprising administrating to said mammal a therapeutically effective amount of the pharmaceutical composition according to claim 25.

38. A method for treating cancerous diseases, infections, or metabolic disorders in a mammal by inhibiting histone deacetylase enzyme comprising administrating to said mammal a therapeutically effective amount of the pharmaceutical composition according to claim 26.

39. The method of claim 31 wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

40. The method of claim 32 wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

41. The method of claim 33 wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

42. The method of claim 34 wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

43. The method of claim 35 wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

44. The method of claim 36 wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

45. The method of claim 37 wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.

46. The method of claim 38 wherein said cancerous disease is angiosarcoma, gastrointestinal stromal tumors (GIST), small cell lung carcinoma (SCLC), thyroid carcinoma, malignant melanoma, adenoid cystic carcinoma, testicular (seminoma), endometrial carcinoma, bladder, breast, ovarian, prostate, colon, rectal, stomach, bronchial, pancreatic, lung, neuroblastoma, head and neck, and gliomas cancer; said metabolic disorder is lymphoma, leukemia, mastocytosis/mast cell leukemia, sinonasal natural killer/T-cell lymphoma, anaplastic large cell lymphoma, hemoglobinopathies, acute myelogenous leukemia (AML), pediatric T-cell acute lymphoblastic, multiple myeloma, cystic fibrosis, and adrenoleukodystrophy; and said infection is malaria, toxoplasmosis, cryptosporoidiosis, trypanosomiasis, or coccidial infection.
